(19) United States
(12) Patent Application Publication
(10) Pub. No.: US 2004/0137581 A1
(43) Pub. Date: Jul. 15, 2004

(54) Interferon variants with improved properties

(75) Inventors: Anna Marie Aguinaldo, Altadena, CA (US); Amelia Joy Beyna, Cheverly, MD (US); Ho Sung Cho, Monrovia, CA (US); John Rudolph Desjarlais, Pasadena, CA (US); Shannon Alicia Marshall, San Francisco, CA (US); Umesh Muchhal, Monrovia, CA (US); Michael Francis Aquino Villegas, Los Angeles, CA (US); Eugene Zhukovsky, West Hollywood, CA (US)

Correspondence Address: Robin M. Silva, Esq., Dorsey & Whitney LLP, Intellectual Property Department, Four Embarcadero Center, Suite 3400, San Francisco, CA, 94111-4187, US

(73) Assignee: Xencor

(21) Appl. No.: 10/676,705

(22) Filed: Sep. 30, 2003

Related U.S. Application Data
(60) Provisional application No. 60/489,725, filed on Jul. 24, 2003.
(60) Provisional application No. 60/477,246, filed on Jun. 10, 2003.
(60) Provisional application No. 60/415,541, filed on Oct. 1, 2002.

(51) Int. Cl. C07K 14/555; C07H 21/04; C12P 21/04

(52) U.S. Cl. 435/69.52; 435/320.1; 435/325; 530/351; 536/23.5

(57) ABSTRACT

The invention relates to interferon variants with improved properties and methods for their use.

This application claims benefit of priority under 35 USC 119(e)(1) to U.S. S. No. 60/415,541, filed Oct. 1, 2002; 60/477,246, filed Jun. 10, 2003 and 60/489,725, filed Jul. 24, 2003, all hereby incorporated by reference in their entirety.

### FIELD OF THE INVENTION

The invention relates to variants of type I interferons with improved properties, and to methods of making compositions utilizing these variants.

### BACKGROUND OF THE INVENTION

Interferons (IFNs) are a well-known family of cytokines possessing a range of biological activities including antiviral, anti-proliferative, and immunomodulatory activities. Interferons have demonstrated utility in the treatment of a variety of diseases, and are in widespread use for the treatment of multiple sclerosis and viral hepatitis.

Interferons include a number of related proteins, such as interferon-alpha (IFN-α), interferon-beta (IFN-β), interferon-gamma (IFN-γ) interferon-kappa (IFN-κ, also known as interferon-epsilon or IFN-ε), interferon-tau (IFN-τ), and interferon-omega (IFN-ω). These interferon proteins are produced in a variety of cell types: IFN-α (leukocytes), IFN-β (fibroblasts), IFN-γ (lymphocytes), IFN-ε or ε (keratinocytes), IFN-ω (leukocytes) and IFN-τ (trophoblasts). IFN-α, IFN-β, IFN-ε or κ, IFN-ω, and IFN-τ are classified as type I interferons, while IFN-γ is classified as a type II interferon. Interferon alpha is encoded by a multi-gene family, while the other interferons appear to each be coded by a single gene in the human genome. Furthermore, there is some allelic variation in interferon sequences among different members of the human population.

Type-I interferons all appear to bind a common receptor, type I IFN-R, composed of IFNAR1 and IFNAR2 subunits. The exact binding mode and downstream signal transduction cascades differ somewhat among the type I interferons. However, in general, the JAK/STAT signal transduction pathway is activated following binding of interferon to the interferon receptor. STAT transcription factors then translocate to the nucleus, leading to the expression of a number of proteins with antiviral, antineoplastic, and immunomodulatory activities.

The properties of naturally occurring type I interferon proteins are not optimal for therapeutic use. Type I interferons induce injection site reactions and a number of other side effects. They are highly immunogenic, eliciting neutralizing and non-neutralizing antibodies in a significant fraction of patients. Interferons are poorly absorbed from the subcutaneous injection site and have short serum half-lives. Finally, type I interferons do not express solubly in prokaryotic hosts, thus necessitating more costly and difficult refolding or mammalian expression protocols.

The present invention is directed to interferon proteins with improved properties. A number of groups have generated modified interferons with improved properties; the references below are all expressly incorporated by reference in their entirety.

Cysteine-depleted variants have been generated to minimize formation of unwanted inter- or intra-molecular disulfide bonds (U.S. Pat. No. 4,518,584; U.S. Pat. No. 4,588,585; U.S. Pat. No. 4,959,314). Methionine-depleted variants have been generated to minimize susceptibility to oxidation (EP 260350).

Interferons with modified activity have been generated (U.S. Pat. No. 6,514,729; U.S. Pat. No. 4,738,844; U.S. Pat. No. 4,738,845; U.S. Pat. No. 4,753,795; U.S. Pat. No. 4,766,106; WO 00/78266). U.S. Pat. Nos. 5,545,723 and 6,127,332 disclose substitution mutants of interferon beta at position 101. Chimeric interferons comprising sequences from one or more interferons have been made (Chang et. al. Nature Biotech. 17: 793-797 (1999), U.S. Pat. No. 4,758,428; U.S. Pat. No. 4,885,166; U.S. Pat. No. 5,382,657; U.S. Pat. No. 5,738,846). Substitution mutations to interferon beta at positions 49 and 51 have also been described (U.S. Pat. No. 6,531,122).

Interferons have been modified by the addition of polyethylene glycol (“PEG”) (see U.S. Pat. No. 4,917,888; U.S. Pat. No. 5,382,657; WO 99/55377; WO 02/09766; WO 02/3114). PEG addition can improve serum half-life and solubility. Serum half-life can also be extended by complexing with monoclonal antibodies (U.S. Pat. No. 5,055,289), by adding glycosylation sites (EP 529300), by co-administering the interferon receptor (U.S. Pat. No. 6,372,207), by preparing single-chain multimers (WO 02/36626) or by preparing fusion proteins comprising an interferon and an immunoglobulin or other protein (WO 01/03737, WO 02/3472, WO 02/36628).

Interferon alpha and interferon beta variants with reduced immunogenicity have been claimed (See WO 02/085941 and WO 02/074783). Due to the large number of variants disclosed and the apparent lack of structural and functional effects of the introduced mutations, identifying a variant that would be a functional, less immunogenic interferon variant suitable for administration to patients may be difficult.

Interferon beta variants with enhanced stability have been claimed, in which the hydrophobic core was optimized using rational design methods (WO 00/68387). Alternate formulations that promote interferon stability or solubility have also been disclosed (U.S. Pat. No. 4,675,483; U.S. Pat. No. 5,730,969; U.S. Pat. No. 5,766,582; WO 02/38170).

Interferon beta muteins with enhanced solubility have been claimed, in which several leucine and phenylalanine residues are replaced with serine, threonine, or tyrosine residues (WO 98/48018). However, due to the lack of support for the specification, it is not clear whether any of the variants claimed are sufficiently soluble, stable, and active to constitute improved variants.

There exists a need for the development and discovery of interferon proteins with improved properties, including but not limited to increased efficacy, decreased side effects, decreased immunogenicity, increased solubility, and enhanced soluble prokaryotic expression. Improved interferon therapeutics may be useful for the treatment of a variety of diseases and conditions, including autoimmune diseases, viral infections, inflammatory diseases, and cancer, among others. In addition, interferons may be used to promote the establishment of pregnancy in certain mammals.

### SUMMARY OF THE INVENTION

The present invention is related to variants of type I human interferons with improved properties, including increased solubility, increased specific activity, and decreased immunogenicity.

### BRIEF DESCRIPTION OF THE DRAWINGS

FIG. 1 shows amino acid sequences for type I interferons.

FIG. 2 shows a sequence alignment of human interferon-alpha subtypes.

FIG. 3 shows the sequence alignment of IFN-α2a (1ITF), IFN-β (1AU1), IFN-κ (IFNK), and IFN-τ (1B5L) that was used to construct the homology model of interferon-kappa.

FIG. 4 shows ISRE assay dose-response curves for interferon beta variants.

FIG. 5 shows a dot blot assay used to test for soluble expression of interferon-kappa variants. G12 and H12 are positive controls, whereas E12 and F12 are soluble extracts from cells expressing WT interferon-kappa (negative control). Wells C5, C8, D4, E5 and F2 represent clones expressing soluble interferon-kappa variants.

FIG. 6 shows a dot blot assay used to test for soluble expression of interferon-kappa variants. G12 and H12 are positive controls, whereas E12 and F12 are soluble extracts from cells expressing WT interferon-kappa (negative control). Most of the putative soluble clones test positive (soluble expression) upon reexpression.

FIG. 7 shows a western blot of solubly expressed interferon kappa variants. The arrow indicates the expected position of interferon-kappa protein. Lanes 2 and 3 are total soluble fraction from WT interferon-kappa expressing cells, respectively. Lanes 4-15 are soluble fractions from the lysates of different variants.

FIG. 8 shows the locations of interferon beta positions 5, 8, 47, 111, and 116 in the context of the dimer structure (PDB code 1AU1). Modifications at these and other positions may disrupt dimerization, thereby increasing the monomeric nature of the protein.

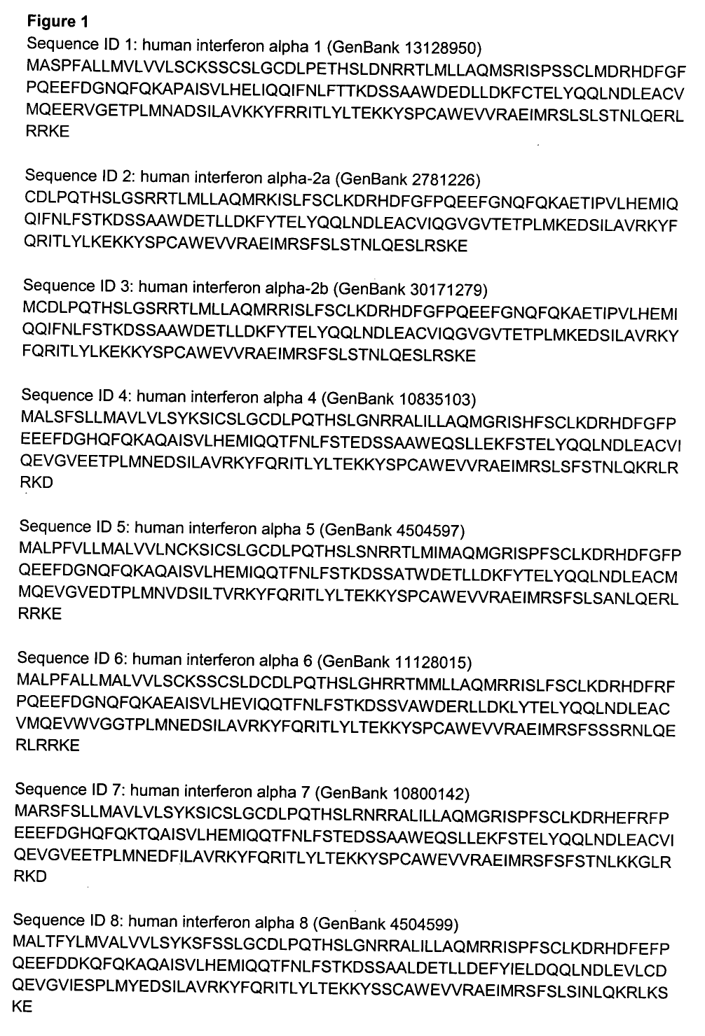

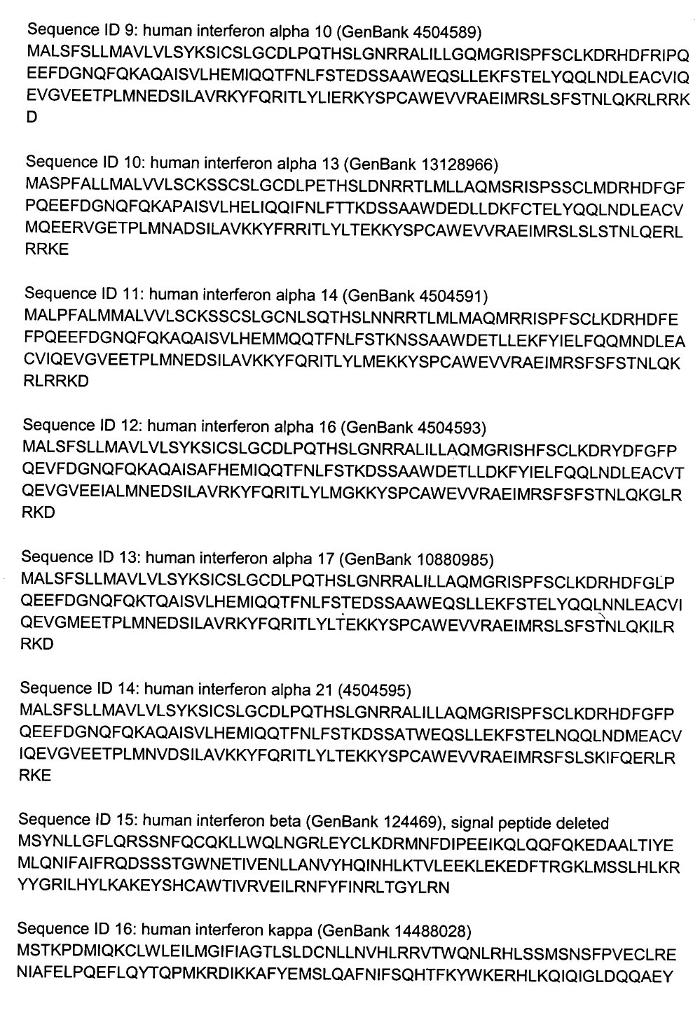

### DETAILED DESCRIPTION OF THE INVENTION

By “control sequences” and grammatical equivalents herein is meant nucleic acid sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. The following residues are defined herein to be “hydrophobic” residues: valine, isoleucine, leucine, methionine, phenylalanine, tyrosine, and tryptophan. By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis. By “reduced immunogenicity” and grammatical equivalents herein is meant a decreased ability to activate the immune system, when compared to the wild type protein. For example, an IFN variant protein can be said to have “reduced immunogenicity” if it elicits neutralizing or non-neutralizing antibodies in lower titer or in fewer patients than wild type IFN. In a preferred embodiment, the probability of raising neutralizing antibodies is decreased by at least 5%, with at least 50% or 90% decreases being especially preferred. Therefore, if a wild type produces an immune response in 10% of patients, a variant with reduced immunogenicity would produce an immune response in not more than 9.5% of patients, with less than 5% or less than 1% being especially preferred. An IFN variant protein also may be said to have “reduced immunogenicity” if it shows decreased binding to one or more MHC alleles or if it induces T-cell activation in a decreased fraction of patients relative to wild type IFN. In a preferred embodiment, the probability of T-cell activation is decreased by at least 5%, with at least 50% or 90% decreases being especially preferred. By “interferon aggregates” and grammatical equivalents herein is meant protein-protein complexes comprising at least one interferon molecule and possessing less immunomodulatory, antiviral, or antineoplastic activity than the corresponding monomeric interferon molecule. Interferon aggregates include interferon dimers, interferon-albumin dimers, higher order species, etc. By “interferon-responsive disorders” and grammatical equivalents herein is meant diseases, disorders, and conditions that can benefit from treatment with a type I interferon. Examples of interferon-responsive disorders include, but are not limited to, autoimmune diseases (e.g. multiple sclerosis, diabetes mellitus, lupus erythematosus, Crohn's disease, rheumatoid arthritis, stomatitis, asthma, allergies and psoriasis), viral infections (e.g. hepatitis C, papilloma viruses, hepatitis B, herpes viruses, viral encephalitis, cytomegalovirus, and rhinovirus), and cell proliferation diseases or cancer (e.g. osteosarcoma, basal cell carcinoma, cervical dysplasia, glioma, acute myeloid leukemia, multiple myeloma, chronic lymphocytic leukemia, Kaposi's sarcoma, chronic myelogenous leukemia, renal-cell carcinoma, ovarian cancers, hairy-cell leukemia, and Hodgkin's disease). Interferons may also be used to promote the establishment of pregnancy in certain mammals. By “library” as used herein is meant a collection of protein sequences that are likely to take on a particular fold or have particular protein properties. The library preferably comprises a set of sequences resulting from computation, which may include energy calculations or statistical or knowledge based approaches. Libraries that range in size from about 50 to about 10¹³ sequences are preferred. Libraries are generally generated experimentally and analyzed for the presence of members possessing desired protein properties. By “modification” and grammatical equivalents is meant insertions, deletions, or substitutions to a protein or nucleic acid sequence. By “naturally occurring” or “wild type” or “wt” and grammatical equivalents thereof herein is meant an amino acid sequence or a nucleotide sequence that is found in nature and includes allelic variations. In a preferred embodiment, the wild-type sequence is the most prevalent human sequence. However, the wild type IFN proteins may be from any number of organisms, include, but are not limited to, rodents (rats, mice, hamsters, guinea pigs, etc.), primates, and farm animals (including sheep, goats, pigs, cows, horses, etc). By “nucleic acid” and grammatical equivalents herein is meant DNA, RNA, or molecules, which contain both deoxy- and ribonucleotides. Nucleic acids include genomic DNA, cDNA and oligonucleotides including sense and anti-sense nucleic acids. Nucleic acids may also contain modifications, such as modifications in the ribose-phosphate backbone that confer increased stability and half-life. Nucleic acids are “operably linked” when placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, elements such as enhancers do not have to be contiguous. A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The following residues are defined herein to be “polar” residues: aspartic acid, asparagine, glutamic acid, glutamine, lysine, arginine, histidine, serine, and threonine. By “protein” herein is meant a molecule comprising at least two covalently attached amino acids, which includes proteins, polypeptides, oligopeptides and peptides. The protein may be made up of naturally occurring amino acids and peptide bonds, or synthetic peptidomimetic structures such as peptoids (see Simon et al., Proc. Natl. Acad. Sci. U.S.A. 89(20:9367-71 (1992)). For example, homo-phenylalanine, citrulline, and noreleucine are considered amino acids for the purposes of the invention, and both D- and L-amino acids may be utilized. By “protein properties” herein is meant biological, chemical, and physical properties including but not limited to enzymatic activity, specificity (including substrate specificity, kinetic association and dissociation rates, reaction mechanism, and pH profile), stability (including thermal stability, stability as a function of pH or solution conditions, resistance or susceptibility to ubiquitination or proteolytic degradation), solubility, aggregation, structural integrity, crystallizability, binding affinity and specificity (to one or more molecules including proteins, nucleic acids, polysaccharides, lipids, and small molecules), oligomerization state, dynamic properties (including conformational changes, allostery, correlated motions, flexibility, rigidity, folding rate), subcellular localization, ability to be secreted, ability to be displayed on the surface of a cell, posttranslational modification (including N- or C-linked glycosylation, lipidation, and phosphorylation), ammenability to synthetic modification (including PEGylation, attachment to other molecules or surfaces), and ability to induce altered phenotype or changed physiology (including cytotoxic activity, immunogenicity, toxicity, ability to signal, ability to stimulate or inhibit cell proliferation, ability to induce apoptosis, and ability to treat disease). When a biological activity is the property, modulation in this context includes both an increase or a decrease in activity. By “solubility” and grammatical equivalents herein is meant the maximum possible concentration of monomeric protein in a solution of specified condition. By “soluble expression” and grammatical equivalents herein is meant that the protein is able to be produced at least partially in soluble form rather than in inclusion bodies when expressed in a prokaryotic host. It is preferred that at least 1 μg soluble protein is produced per 100 mL culture, with at least 10 μg or 100 μg being especially preferred. By “improved solubility” and grammatical equivalents herein is meant an increase in the maximum possible concentration of monomeric protein in solution. For example, if the naturally occurring protein can be concentrated to 1 mM and the variant can be concentrated to 5 mM under the same solution conditions, the variant can be said to have improved solubility. In a preferred embodiment, solubility is increased by at least a factor of 2, with increases of at least 5× or 10× being especially preferred. As will be appreciated by those skilled in the art, solubility is a function of solution conditions. For the purposes of this invention, solubility should be assessed under solution conditions that are pharmaceutically acceptable. Specifically, pH should be between 6.0 and 8.0, salt concentration should be between 50 and 250 mM. Additional buffer components such as excipients may also be included, although it is preferred that albumin is not required. By “therapeutically effective dose” herein is meant a dose that produces the effects for which it is administered. The exact dose will depend on the purpose of the treatment, and will be ascertainable by one skilled in the art using known techniques. As is known in the art, adjustments for variant IFN protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. By “treatment” herein is meant to include therapeutic treatment, as well as prophylactic, or suppressive measures for the disease or disorder. Thus, for example, successful administration of a variant IFN protein prior to onset of the disease may result in treatment of the disease. As another example, successful administration of a variant IFN protein after clinical manifestation of the disease to combat the symptoms of the disease comprises “treatment” of the disease. “Treatment” also encompasses administration of a variant IFN protein after the appearance of the disease in order to eradicate the disease. Successful administration of an agent after onset and after clinical symptoms have developed, with possible abatement of clinical symptoms and perhaps amelioration of the disease, further comprises “treatment” of the disease. By “variant interferon nucleic acids” and grammatical equivalents herein is meant nucleic acids that encode variant interferon proteins. Due to the degeneracy of the genetic code, an extremely large number of nucleic acids may be made, all of which encode the variant interferon proteins of the present invention, by simply modifying the sequence of one or more codons in a way that does not change the amino acid sequence of the variant interferon. By “variant interferon proteins” or “non-naturally occurring interferon proteins” and grammatical equivalents thereof herein is meant non-naturally occurring interferon proteins which differ from the wild type interferon protein by at least one (1) amino acid insertion, deletion, or substitution. It should be noted that unless otherwise stated, all positional numbering of variant interferon proteins and variant interferon nucleic acids is based on these sequences. Interferon variants are characterized by the predetermined nature of the variation, a feature that sets them apart from naturally occurring allelic or interspecies variation of the interferon protein sequence. The interferon variants must retain at least 50% of wild type interferon activity, as determined using the ISRE assay described below. Variants that retain at least 75% or 90% of wild type activity are more preferred, and variants that are more active than wild type are especially preferred. The variant interferon proteins may contain insertions, deletions, and/or substitutions at the N-terminus, C-terminus, or internally. In a preferred embodiment, variant IFN proteins have at least 1 residue that differs from the most similar human interferon sequence, with at least 2, 3, 4, or 5 different residues being more preferred. Variant interferon proteins may contain further modifications, for instance mutations that alter additional protein properties such as stability or immunogenicity or which enable or prevent posttranslational modifications such as PEGylation or glycosylation. Variant interferon proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels.

Naturally occurring interferons possess antiviral, antiproliferative, and immunomodulatory activities, making interferons valuable therapeutics. However, drugs based on naturally occurring interferons suffer from a number of liabilities, including a high incidence of side effects and immunogenicity.

Here, are disclosed novel variants of type I interferon proteins. These interferon variants comprise one or more modifications that were selected to improve biophysical properties and clinical performance. Poor solubility contributes to many of the liabilities of current interferon therapeutics. Accordingly, a primary focus of this invention is interferon variants with improved solubility.

Although type I interferons are biologically active as monomers, they are known to form dimers and higher order species. These species may consist primarily of interferon proteins, or may also contain additional proteins such as human serum albumin. Non-monomeric interferon species exhibit significantly decreased activity, as even dimer formation interferes with receptor binding (Utsumi et. al. Biochim. Biophys. Acta 998: 167 (1989) and Runkel et. al. Pharm. Res. 15: 641 (1998)). Interferon therapeutics are known to elicit neutralizing antibodies in a substantial fraction of patients (Antonelli et. al. Eur. Cytokine Netw. 10: 413 (1999)). Poor solubility may be a significant contributing factor to the immunogenicity of interferon therapeutics, as aggregates are typically more immunogenic than soluble proteins (Speidel et. al. Eur. J. Immunol. 27: 2391 (1997)), and aggregation has been demonstrated to increase the immunogenicity of interferon-alpha (Braun et. al. Pharm. Res. 14: 1472 (1997)). Furthermore, poor solubility results in reduced absorption following subcutaneous injection (Clodfelter et. al. Pharm. Res. 15: 254 (1998)).

A variety of strategies may be utilized to design IFN variants with improved solubility. In a preferred embodiment, one or more of the following strategies are used: 1) reduce hydrophobicity by substituting one or more solvent-exposed hydrophobic residues with suitable polar residues, 2) increase polar character by substituting one or more neutral polar residues with charged polar residues, 3) decrease formation of intermolecular disulfide bonds by modifying one or more non-disulfide bonded cysteine residues (unpaired cysteines), 4) reduce the occurrence of known unwanted protein-protein interactions by modifying one or more residues located at protein-protein interaction sites such as dimer interfaces, 5) increase protein stability, for example by one or more modifications that improve packing in the hydrophobic core, improve helix capping and dipole interactions, or remove unfavorable electrostatic interactions, and 6) modify one or more residues that can affect the isoelectric point of the protein (that is, aspartic acid, glutamic acid, histidine, lysine, arginine, tyrosine, and cysteine residues) to decrease the isoelectric point of the protein below physiological pH. Increasing the stability of a protein may improve solubility by decreasing the population of partially folded or misfolded states. Protein solubility is typically at a minimum when the isoelectric point of the protein is equal to the pH of the surrounding solution. Modifications that perturb the isoelectric point of the protein away from the pH of a relevant environment, such as serum, may therefore serve to improve solubility. Furthermore, modifications that decrease the isoelectric point of a protein may improve injection site absorption (Holash et. al. PNAS 99: 11393-11398 (2002)).

Type I interferons typically have one free cysteine residue and several exposed hydrophobic residues. These positions can be targeted for mutagenesis in order to improve solubility. Replacing exposed hydrophobic residues with appropriate polar residues may also decrease the number of MHC-binding epitopes. (See U.S. Ser. No. 10/039,170, filed Jan. 8, 2003) Since MHC binding is a key step in the initiation of an immune response, such mutations may decrease immunogenicity by multiple mechanisms.

In two cases, type I interferons have been observed to crystallize as dimers or higher order species. While the dimeric structure is significantly less active than the monomer, it may represent a species that is present in interferon therapeutics. Accordingly, residues located at or close to the protein-protein interfaces can be targeted for modification.

A number of methods can be used to identify modifications (that is, insertion, deletion, or substitution mutations) that will yield interferon variants with improved solubility and retained or improved immunomodulatory, antiviral, or antineoplastic activity. These include, but are not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994); and residue pair potentials (Jones, Protein Science 3: 567-574, (1994).

In an especially preferred embodiment, rational design of improved IFN variants is achieved by using Protein Design Automation® (PDA®) technology. (See U.S. Pat. Nos. 6,188,965; 6,269,312; 6,403,312; WO98/47089 and U.S. Ser. Nos. 09/058,459, 09/127,926, 60/104,612, 60/158,700, 09/419,351, 60/181,630, 60/186,904, 09/419,351, 09/782,004 and 09/927,790, 60/347,772, and 10/218,102; and PCT/US01/218,102 and U.S. Ser. No. 10/218,102, U.S. S. No. 60/345,805; U.S. S. No. 60/373,453 and U.S. S. No. 60/374,035, all references expressly incorporated herein in their entirety.)

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions.

In a preferred embodiment, each polar residue is represented using a set of discrete low-energy side-chain conformations (see for example Dunbrack Curr. Opin. Struct. Biol. 12:431-440 (2002). A preferred force field may include terms describing van der Waals interactions, hydrogen bonds, electrostatic interactions, and salvation, among others.

In a preferred embodiment, Dead-End Elimination (DEE) is used to identify the rotamer for each polar residue that has the most favorable energy (see Gordon et. al. J. Comput Chem. 24: 232-243 (2003), Goldstein Biophys. J. 66: 1335-1340 (1994) and Lasters and Desmet, Prot. Eng. 6: 717-722 (1993)).

In an alternate embodiment, Monte Carlo can be used in conjunction with DEE to identify groups of polar residues that have favorable energies.

In a preferred embodiment, after performing one or more PDA® technology calculations, a library of variant proteins is designed, experimentally constructed, and screened for desired properties.

In an alternate preferred embodiment, a sequence prediction algorithm (SPA) is used to design proteins that are compatible with a known protein backbone structure as is described in Raha, K., et al. (2000) Protein Sci., 9: 1106-1119; U.S. Ser. No. 09/877,695, filed Jun. 8, 2001 and Ser. No. 10/071,859, filed Feb. 6, 2002.

In one embodiment, the library is a combinatorial library, meaning that the library comprises all possible combinations of allowed residues at each of the variable positions. For example, if positions 3 and 9 are allowed to vary, allowed choices at position 3 are A, V, and 1, and allowed choices at position 9 are E and Q, the library includes the following three variant sequences: 3A/9E, 3A/9Q, 3V/9E, 3V/9Q, 3I/9E, and 3I/9Q.

Obtaining Structures of Type I Interferons

PDA® technology calculations, described above, require a template protein structure. In a most preferred embodiment, the structure of a type I interferon is obtained by solving its crystal structure or NMR structure by techniques well known in the art. High-resolution structures are available for type I interferons including interferon-α2a (interferon-alpha2a), interferon-α2b (interferon-alpha2b), interferon-β (interferon-beta), and interferon-τ (interferon-tau) (see Radhakrishnan et. al. J. Mol. Biol. 286:151-162 (1999), Karpusas et. al. Proc. Nat. Acad. Sci. USA 94:22 (1997), Klaus et. al. J. Mol. Biol. 274:661-675 (1997), Radhakrishnan et. al. Structure 4:1453-1463 (1996)).

In an alternate embodiment, a homology model is built, using methods known to those in the art. Homology models of interferons have been constructed previously, see for example Seto et. al. Protein Sci. 4:655-670 (1995).

Identifying Solvent-Exposed Hydrophobic Residue Positions

Hydrophobic residues as used herein may be valine, leucine, isoleucine, methionine, phenylalanine, tyrosine, and tryptophan. Exposed residues as used herein as those residues whose side chains have at least 30 Å² (square Angstroms) of solvent accessible surface area. As will be appreciated by those skilled in the art, other values such as 50 Å² (square Angstroms) or fractional values such as 50% could be used instead. Furthermore, alternative methods such as contact models, among others, may be used to identify exposed residues.

As used herein, for example, solvent exposed hydrophobic residues in interferon-alpha 2a include, but are not limited to, Met 16, Phe 27, Leu 30, Tyr 89, Ile 100, Leu 110, Met 111, Leu 117, Leu 128, and Leu 161.

Especially preferred solvent exposed hydrophobic residues are those that have not been implicated in interferon alpha function or receptor binding (see for example Piehler et. al. J. Biol. Chem. 275: 40425-40433 (2000), Hu et. al. J. Immunol. 163: 854-860 (1999), Hu et. al. J. Immunol. 167: 1482-1489 (2001)), including Met 16, Phe 27, Ile 100, Leu 110, Met 111, Leu 117, and Leu 161.

As used herein, for example, solvent exposed hydrophobic residues in interferon-beta include, but are not limited to, Leu 5, Phe 8, Phe 15, Trp 22, Leu 28, Tyr 30, Leu 32, Met 36, Leu 47, Tyr 92, Phe 111, Leu 116, Leu 120, Leu 130, Val 148, and Tyr 155.

Especially preferred solvent exposed hydrophobic residues are those residues that have not been implicated in interferon beta function or receptor binding (see for example Runkel et. al. Biochem. 39: 2538-2551 (2000), Runkel et. al. J. Int. Cytokine Res. 21: 931-941 (2001)), include Leu 5, Phe 8, Leu 47, Phe 111, Leu 116, and Leu 120.

As used herein, for example, solvent exposed hydrophobic residues in interferon-kappa include, but are not limited to, Leu 1, Leu 5, Val 8, Trp 15, Leu 18, Phe 28, Val 30, Leu 33, Ile 37, Leu 46, Tyr 48, Met 52, Leu 65, Phe 68, Phe 76, Tyr 78, Trp 79, lie 89, Tyr 97, Met 112, Met 115, Met 120, Val 127, Leu 133, Tyr 151, Val 161, Tyr 168, and Tyr 171.

Especially preferred solvent exposed hydrophobic residues are located at positions that are polar in other interferon sequences, and include Leu 5, Val 8, Trp 15, Phe 28, Val 30, Ile 37, Tyr 48, Met 52, Phe 76, Tyr 78, Ile 89, Tyr 97, Val 161, Tyr 168, and Tyr 171.

Identifying Unpaired Cysteine Positions

Unpaired cysteines are defined to be cysteines that do not form a disulfide bond in the folded protein. Unpaired cysteines can be identified, for example, by visual analysis of the structure or by analysis of the disulfide bond patterns of related proteins.

Interferon alpha-1 and interferon alpha-13 contain one unpaired cysteine at position 86 (Cys 86).

Interferon-beta contains one unpaired cysteine at position 17 (Cys 17).

Interferon-kappa contains one unpaired cysteine at position 166 (Cys 166).

Ovine interferon-tau contains one unpaired cysteine at position 86 (Cys 86).

Identifying Dimer Interface Residues

In a preferred embodiment, residues that mediate intermolecular interactions between interferon monomers or between interferon and human serum albumin are replaced with structurally and functionally compatible residues that confer decreased propensity for unwanted intermolecular interactions.

In a preferred embodiment, interface residues are defined as those residues located within 8 Å (Angstroms) of a protein-protein contact. Distances of less than 5 Å (Angstroms) are especially preferred. Distances may be measured using any structure with high-resolution crystal structures being especially preferred.

Preferred interface residues in interferon alpha include, but are not limited to, residues 16, 19, 20, 25, 27, 28, 30, 33, 35-37, 39-41, 44-46, 54, 58, 61, 65, 68, 85, 91, 99, 112-115, 117, 118, 121, 122, 125, and 149.

Preferred interface residues in interferon beta include, but are not limited to, residues 1-6, 8, 9, 12, 16, 42, 43, 46, 47, 49, 51, 93, 96, 97, 100, 101, 104, 113, 116, 117, 120, 121, and 124.

Identifying Suitable Polar Residues for Each Exposed Hydrophobic Position

In a preferred embodiment, solvent exposed hydrophobic residues are replaced with structurally and functionally compatible polar residues. As used herein, polar residues include serine, threonine, histidine, aspartic acid, asparagine, glutamic acid, glutamine, arginine, and lysine. Alanine and glycine may also serve as suitable replacements, constituting a reduction in hydrophobicity.

In a preferred embodiment, suitable polar residues include only the subset of polar residues that are observed in analogous positions in homologous proteins, especially other interferons.

In a preferred embodiment, preferred suitable polar residues are defined as those polar residues: 1) Whose energy in the optimal rotameric configuration is more favorable than the energy of the exposed hydrophobic residue at that position and 2) Whose energy in the optimal rotameric configuration is among the most favorable of the set of energies of all polar residues at that position.

In a preferred embodiment, the BLAST alignment algorithm is used to generate alignments proteins that are homologs of an interferon of interest. Examples of homologous proteins include other classes of type I interferons, allelic variants of interferon, and interferons from other species.

In a preferred embodiment, the frequency of occurrence of each polar residue at each position is normalized using the method of Henikoff & Henikoff (J. Mol. Biol. 243: 547-578 (1994)). In an alternate embodiment, a simple count of the number of occurrences of each polar residue at each position is made.

In a preferred embodiment, the polar residues that are included in the library at each variable position are deemed suitable by both PDA® technology calculations and by sequence alignment data. Alternatively, one or more of the polar residues that are included in the library are deemed suitable by either PDA® technology calculations or sequence alignment data.

In a preferred embodiment, residues that are close in sequence are “coupled” in the library, meaning that all combinatorial possibilities are not sampled. For instance, if the library includes residues L and Q at position 5 and residues F and E at position 8, a “coupled” library could include L5/F8 and Q5/E8 but not include L5/E8 or Q5/F8. Coupling residues decreases the overall combinatorial complexity of the library, thereby simplifying screening. Furthermore, coupling can be used to avoid the introduction of two or more modifications that are incompatible with each other.

Especially preferred modifications to interferon-alpha include, but are not limited to, M16D, F27Q, I100Q, L110N, M111Q, L117R, and L161E.

Especially preferred modifications to interferon-beta include, but are not limited to, L5Q, F8E, F111N, L116E, and L120R.

Especially preferred modifications to interferon-kappa include, but are not limited to, L5Q, V8N, W15R, F28Q, V30R, I37N, Y48Q, M52N, F76S, Y78A, I89T, Y97D, M112T, M115G, L133Q, V161A, Y168S, and Y171T.

Identifying Suitable Residues for Each Interface Position

Suitable residues for interface residues as used herein are meant all amino acid residues that are compatible with the structure and function of a type I interferon, but which are substantially incapable of forming unwanted intermolecular interactions, including but not limited to interactions with other interferon molecules and interactions with human serum albumin.

Typically, the interface positions will be substantially exposed to solvent. In such cases, preferred substitutions include alanine and the polar residues serine, threonine, histidine, aspartic acid, asparagine, glutamic acid, glutamine, arginine, and lysine. However, for interface positions that are substantially buried in the monomer structure, hydrophobic replacements are preferred.

In a preferred embodiment, suitable polar residues include only the subset of polar residues that are observed in analogous positions in homologous proteins, especially other interferons, that do not form a given unwanted intermolecular interaction.

In an especially preferred embodiment, suitable polar residues include only the subset of polar residues with low or favorable energies as determined using PDA® technology calculations or SPA calculations (described above).

In a most especially preferred embodiment, suitable polar residues include only the subset of polar residues that are determined to be compatible with the monomer structure and incompatible with a given unwanted intermolecular interaction, as determined using PDA® technology calculations or SPA calculations.

Especially preferred modifications to interferon-beta include L5A, L5D, L5E, L5K, L5N, L5Q, L5R, L5S, L5T, F8A, F8D, F8E, F8K, F8N, F8Q, F8R, F8S, S12E, S12K, S12Q, S12R, E43K, E43R, R113D, L116D, L116E, L116N, L116Q, L116R, and M117R.

Identifying Suitable Non-Cysteine Residues for Each Unpaired Cysteine Position

Suitable non-cysteine residues as used herein are meant all amino acid residues other than cysteine. In a preferred embodiment, if the cysteine position is substantially buried in the protein core, suitable non-cysteine residues include alanine and the hydrophobic residues valine, leucine, isoleucine, methionine, phenylalanine, tyrosine, and tryptophan.

In a preferred embodiment, if the cysteine position is substantially exposed to solvent, suitable non-cysteine residues include alanine and the polar residues serine, threonine, histidine, aspartic acid, asparagine, glutamic acid, glutamine, arginine, and lysine.

In a preferred embodiment, suitable residues are defined as those with low (favorable) energies as calculated using PDA® technology.

In a preferred embodiment, suitable residues defined as those that are observed at analogous positions in other interferon proteins. For example, position 86 is an unpaired cysteine in some interferon-alpha1 and interferon-alpha13, but is replaced with tyrosine or serine in other interferon alpha subtypes. Also, position 166 is an unpaired cysteine in interferon-kappa, but is frequently alanine in other interferon sequences.

In a more preferred embodiment, suitable residues are those that have both low (favorable) energies as calculated using PDA® technology and are observed in the analogous position in other interferon proteins.

In a most preferred embodiment, Cys 86 in interferon-alpha 1 or interferon alpha-13 replaced by glutamic acid, lysine, or glutamine.

In a most preferred embodiment, Cys 17 in interferon-beta is replaced by alanine, aspartic acid, asparagine, serine or threonine.

In a most preferred embodiment, Cys 166 in interferon-kappa is replaced by alanine, glutamic acid, or histidine.

Additional Modifications

Additional insertions, deletions, and substitutions may be incorporated into the variant interferon proteins of the invention in order to confer other desired properties.

In a preferred embodiment, the immunogenicity of interferons may be modulated. See for example U.S. Ser. Nos. 09/903,378; 10/039,170; 10/339,788 (filed Jan. 8, 2003, titled Novel Protein with Altered Immunogenicity); and PCT/US01/21823; and PCT/US02/00165. All references expressly incorporated by reference in their entirety.

In an alternate preferred embodiment, the interferon variant is further modified to increase stability. As discussed above, modifications that improve stability can also improve solubility, for example by decreasing the concentration of partially unfolded, aggregation-prone species. For example, modifications can be introduced to the protein core that improve packing or remove polar or charged groups that are not forming favorable hydrogen bond or electrostatic interactions. It is also possible to introduce modifications that introduce stabilizing electrostatic interactions or remove destabilizing interactions. Additional stabilizing modifications also may be used.

In one embodiment, the sequence of the variant interferon protein is modified in order to add or remove one or more N-linked or O-linked glycosylation sites. Addition of glycosylation sites to variant interferon polypeptides may be accomplished, for example, by the incorporation of one or more serine or threonine residues to the native sequence or variant interferon polypeptide (for O-linked glycosylation sites) or by the incorporation of a canonical N-linked glycosylation site, N-X-Y, where X is any amino acid except for proline and Y is threonine, serine or cysteine. Glycosylation sites may be removed by replacing one or more serine or threonine residues or by replacing one or more N-linked glycosylation sites.

In another preferred embodiment, one or more cysteine, lysine, histidine, or other reactive amino acids are designed into variant interferon proteins in order to incorporate labeling sites or PEGylation sites. It is also possible to remove one or more cysteine, lysine, histidine, or other reactive amino acids in order to prevent the incorporation of labeling sites or PEGylations sites at specific locations. For example, in a preferred embodiment, non-labile PEGylation sites are selected to be well removed from any required receptor binding sites in order to minimize loss of activity.

Variant interferon polypeptides of the present invention may also be modified to form chimeric molecules comprising a variant interferon polypeptide fused to another, heterologous polypeptide or amino acid sequence. In one embodiment, such a chimeric molecule comprises a fusion of a variant interferon polypeptide with a tag polypeptide which provides an epitope to which an anti-tag antibody can selectively bind. The epitope tag is generally placed at the amino-or carboxyl-terminus of the variant interferon polypeptide. The presence of such epitope-tagged forms of a variant interferon polypeptide can be detected using an antibody against the tag polypeptide. Also, provision of the epitope tag enables the variant interferon polypeptide to be readily purified by affinity purification using an anti-tag antibody or another type of affinity matrix that binds to the epitope tag. Various tag polypeptides and their respective antibodies are well known in the art. Examples include poly-histidine (poly-His) or poly-histidine-glycine (poly-His-Gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6): 547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

In an alternative embodiment, the chimeric molecule may comprise a fusion of a variant interferon polypeptide with another protein. Various fusion partners are well known in the art, and include but are not limited to the following examples. The variant interferon proteins of the invention may be fused to an immunoglobulin or the Fc region of an immunoglobulin, such as an IgG molecule. The interferon variants can also be fused to albumin, other interferon proteins, other cytokine proteins, the extracellular domains of the interferon receptor protein, etc.

In another embodiment, the N- and C-termini of a variant IFN protein are joined to create a cyclized or circularly permutated IFN protein. Various techniques may be used to permutate proteins. See U.S. Pat. No. 5,981,200; Maki K, Iwakura M., Seikagaku. 2001 January; 73(1): 42-6; Pan T., Methods Enzymol. 2000; 317:313-30; Heinemann U, Hahn M., Prog Biophys Mol Biol. 1995; 64(2-3): 121-43; Harris M E, Pace N R, Mol Biol Rep. 1995-96; 22(2-3):115-23; Pan T, Uhlenbeck O C., 1993 Mar. 30; 125(2): 111-4; Nardulli A M, Shapiro D J. 1993 Winter; 3(4):247-55, EP 1098257 A2; WO 02/22149; WO 01/51629; WO 99/51632; Hennecke, et al., 1999, J. Mol. Biol., 286, 1197-1215; Goldenberg et al J. Mol. Biol 165, 407-413 (1983); Luger et al, Science, 243, 206-210 (1989); and Zhang et al., Protein Sci 5, 1290-1300 (1996); all hereby incorporated by reference.

To produce a circularly permuted IFN protein, a novel set of N- and C-termini are created at amino acid positions normally internal to the protein's primary structure, and the original N- and C-termini are joined via a peptide linker consisting of from 0 to 30 amino acids in length (in some cases, some of the amino acids located near the original termini are removed to accommodate the linker design). In a preferred embodiment, the novel N- and C-termini are located in a non-regular secondary structural element, such as a loop or turn, such that the stability and activity of the novel protein are similar to those of the original protein. The circularly permuted IFN protein may be further PEGylated, glycosylated, or otherwise modified. In a further preferred embodiment PDA® technology may be used to further optimize the IFN variant, particularly in the regions affected by circular permutation. These include the novel N- and C-termini, as well as the original termini and linker peptide.

In addition, a completely cyclic IFN may be generated, wherein the protein contains no termini. This is accomplished utilizing intein technology. Thus, peptides can be cyclized and in particular inteins may be utilized to accomplish the cyclization.

Generating the Variants

Variant interferon nucleic acids and proteins of the invention may be produced using a number of methods known in the art.

Preparing Nucleic Acids Encoding the IFN Variants

In a preferred embodiment, nucleic acids encoding IFN variants are prepared by total gene synthesis, or by site-directed mutagenesis of a nucleic acid encoding wild type or variant IFN protein. Methods including template-directed ligation, recursive PCR, cassette mutagenesis, site-directed mutagenesis or other techniques that are well known in the art may be utilized (see for example Strizhov et. al. PNAS 93:15012-15017 (1996), Prodromou and Perl, Prot. Eng. 5: 827-829 (1992), Jayaraman and Puccini, Biotechniques 12: 392-398 (1992), and Chalmers et. at. Biotechniques 30: 249-252 (2001)).

Expression Vectors

In a preferred embodiment, an expression vector that comprises the components described below and a gene encoding a variant IFN protein is prepared. Numerous types of appropriate expression vectors and suitable regulatory sequences for a variety of host cells are known in the art. The expression vectors may contain transcriptional and translational regulatory sequences including but not limited to promoter sequences, ribosomal binding sites, transcriptional start and stop sequences, translational start and stop sequences, transcription terminator signals, polyadenylation signals, and enhancer or activator sequences. In a preferred embodiment, the regulatory sequences include a promoter and transcriptional start and stop sequences. In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences, which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used. The expression vectors may be either self-replicating extrachromosomal vectors or vectors which integrate into a host genome.

The expression vector may include a secretory leader sequence or signal peptide sequence that provides for secretion of the variant IFN protein from the host cell. Suitable secretory leader sequences that lead to the secretion of a protein are known in the art. The signal sequence typically encodes a signal peptide comprised of hydrophobic amino acids, which direct the secretion of the protein from the cell. The protein is either secreted into the growth media or, for prokaryotes, into the periplasmic space, located between the inner and outer membrane of the cell. For expression in bacteria, bacterial secretory leader sequences, operably linked to a variant IFN encoding nucleic acid, are usually preferred.

Transfection/Transformation

The variant IFN nucleic acids are introduced into the cells either alone or in combination with an expression vector in a manner suitable for subsequent expression of the nucleic acid. The method of introduction is largely dictated by the targeted cell type. Exemplary methods include CaPO₄ precipitation, liposome fusion, Lipofectin®, electroporation, viral infection, dextran-mediated transfection, polybrene mediated transfection, protoplast fusion, direct microinjection, etc. The variant IFN nucleic acids may stably integrate into the genome of the host cell or may exist either transiently or stably in the cytoplasm. As outlined herein, a particularly preferred method utilizes retroviral infection, as outlined in PCT/US97/01019, incorporated by reference.

Hosts for the Expression of IFN Variants

Appropriate host cells for the expression of IFN variants include yeast, bacteria, archaebacteria, fungi, and insect and animal cells, including mammalian cells. Of particular interest are bacteria such as E. coli and Bacillus subtilis, fungi such as Saccharomyces cerevisiae, Pichia pastoris, and Neurospora, insects such as Drosophila melangaster and insect cell lines such as SF9, mammalian cell lines including 293, CHO, COS, Jurkat, NIH3T3, etc (see the ATCC cell line catalog, hereby expressly incorporated by reference), as well as primary cell lines.

Interferon variants can also be produced in more complex organisms, including but not limited to plants (such as corn, tobacco, and algae) and animals (such as chickens, goats, cows); see for example Dove, Nature Biotechnol. 20: 777-779 (2002).

In one embodiment, the cells may be additionally genetically engineered, that is, contain exogenous nucleic acid other than the expression vector comprising the variant IFN nucleic acid.

Expression Methods

The variant IFN proteins of the present invention are produced by culturing a host cell transformed with an expression vector containing nucleic acid encoding a variant IFN protein, under the appropriate conditions to induce or cause expression of the variant IFN protein. The conditions appropriate for variant IFN protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Purification

In a preferred embodiment, the IFN variants are purified or isolated after expression. Standard purification methods include electrophoretic, molecular, immunological and chromatographic techniques, including ion exchange, hydrophobic, affinity, and reverse-phase HPLC chromatography, and chromatofocusing. For example, a IFN variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3d ed. (1994). The degree of purification necessary will vary depending on the desired use, and in some instances no purification will be necessary. For further references on purification of type I interferons, see for example Moschera et al. Meth. Enzym. 119: 177-183 (1986); Tarnowski et al. Meth. Enzym. 119:153-165(1986); Thatcher et al. Meth. Enzym. 119:166-177(1986); Lin et al. Meth. Enzym. 119:183-192(1986). Methods for purification of interferon beta are disclosed in U.S. Pat. No. 4,462,940 and U.S. Pat. No. 4,894,330.

Posttranslational Modification and Derivitization

Once made, the variant IFN proteins may be covalently modified. Covalent and non-covalent modifications of the protein are thus included within the scope of the present invention. Such modifications may be introduced into a variant IFN polypeptide by reacting targeted amino acid residues of the polypeptide with an organic derivatizing agent that is capable of reacting with selected side chains or terminal residues. Optimal sites for modification can be chosen using a variety of criteria, including but not limited to, visual inspection, structural analysis, sequence analysis and molecular simulation.

In one embodiment, the variant IFN proteins of the invention are labeled with at least one element, isotope or chemical compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position. Labels include but are not limited to biotin, tag (e.g. FLAG, Myc) and fluorescent labels (e.g. fluorescein).

Derivatization with bifunctional agents is useful, for instance, for cross linking a variant IFN protein to a water-insoluble support matrix or surface for use in the method for purifying anti-variant IFN antibodies or screening assays, as is more fully described below. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Such derivitization may improve the solubility, absorption, permeability across the blood brain barrier, serum half life, and the like. Modifications of variant IFN polypeptides may alternatively eliminate or attenuate any possible undesirable side effect of the protein. Moieties capable of mediating such effects are disclosed, for example, in Remington's Pharmaceutical Sciences, 16th ed., Mack Publishing Co., Easton, Pa. (1980).

Another type of covalent modification of variant IFN comprises linking the variant IFN polypeptide to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 or 4,179,337. A variety of coupling chemistries may be used to achieve PEG attachment, as is well known in the art. Examples, include but are not limited to, the technologies of Shearwater and Enzon, which allow modification at primary amines, including but not limited to, cysteine groups, histidine groups, lysine groups and the N-terminus (see, Kinstler et al, Advanced Drug Deliveries Reviews, 54, 477-485 (2002) and M J Roberts et al, Advanced Drug Delivery Reviews, 54, 459-476 (2002)). Both labile and non-labile PEG linkages may be used.

An additional form of covalent modification includes coupling of the variant IFN polypeptide with one or more molecules of a polymer comprised of a lipophililic and a hydrophilic moiety. Such composition may enhance resistance to hydrolytic or enzymatic degradation of the IFN protein. Polymers utilized may incorporate, for example, fatty acids for the lipophilic moiety and linear polyalkylene glycols for the hydrophilic moiety. The polymers may additionally incorporate acceptable sugar moieties as well as spacers used for IFN protein attachment. Polymer compositions and methods for covalent conjugation are described, for example, in U.S. Pat. Nos. 5,681,811; 5,359,030.

Another type of modification is chemical or enzymatic coupling of glycosides to the variant IFN protein. Such methods are described in the art, e.g., in WO 87/05330 published 11 Sep. 1987, and in Aplin and Wriston, CRC Crit. Rev. Biochem., pp. 259-306 (1981).

Alternatively, removal of carbohydrate moieties present on the variant IFN polypeptide may be accomplished chemically or enzymatically. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Assaying the Solubility of the Variants

A primary object of the invention is the identification of variant interferon proteins with improved solubility. Accordingly, in a preferred embodiment, the variant interferon proteins are assayed for solubility using methods including but not limited to those described below.

In all preferred embodiments, the variant and wild type proteins are compared directly in the same assay system and under the same conditions in order to evaluate the solubility of each variant.

The solubility of the interferon variant proteins may be determined under a number of solution conditions. A variety of excipients, including solubilizing and stabilizing agents, may be tested for their ability to promote the highest stable IFN concentration. In addition, different salt concentrations and varying pH may be tested. In a preferred embodiment, solubility is assayed under pharmaceutically acceptable conditions.

In a preferred embodiment, differential light scattering (DLS) is used to determine oligomerization state. DLS determines diffusion coefficients based on signal correlation from fluctuation of laser light scattered from Brownian motion of particles in solution (Heimenz, Chapter 10 in Polymer Chemistry, Marcel Dekker, Inc., NY, 1984, pp. 659-701). Commercially available instruments provide graphical or table readouts of particle population(s) by size(s) after transforming the diffusion coefficient(s) measured by deconvolution/autocorrelation of laser light scattering data using the Stokes-Einstein equation. The size is therefore the hydrodynamic radius. Particle size standards may be used to check the accuracy of the instrument settings (nanoparticles obtained from Duke Scientific Corporation, Palo Alto, Calif.). The distribution of particle sizes within a population(s) is the dispersity, and this factor provides data on the uniformity of the particle population(s). Both dispersity and the appearance of aggregates over time may be monitored to test for solubility.

Aggregated protein may be easily resolved by DLS, and readily detected at low levels due to the physical property of aggregates: they scatter more laser light per unit due to the greater target surface area. The sample may be directly introduced into the cuvette (i.e. it is not necessary to perform a chromatographic step first). A relative ratio of monodisperse to aggregate particle population may be determined. Optionally, this ratio may be weighted by mass or by light scattering intensity. Thus, DLS is a preferred technique to monitor formation of aggregates, and holds the advantage in that it is a non-intrusive technique.

In another preferred embodiment analytical ultracentrifugation (AUC) is used to determine the oligomerization state of the variant interferon proteins. AUC can be performed in two different ‘modes’, either velocity or equilibrium. Equilibrium AUC is the most preferred method for determining protein molecular weight and oligomeric state measurement.

A further preferred embodiment is to use size-exclusion chromatography (SEC) to determine the oligomerization state of the variant interferon proteins. Utilizing high performance liquid chromatography, sample may be introduced to an isocratic mobile phase and separated on a gel permeation matrix designed to exclude protein on the basis of size. Thus, the samples will be “sieved” such that the aggregated protein will elute first with the shortest retention time, and will be easily separated from the remainder. This can identify aggregates and allow a relative quantification by peak integration using the peak analysis software provided with the instrument.

In an alternate embodiment, protein concentration is monitored as a function of time. In the case of poor solubility, aggregates will form over time in the protein solution, and eventually precipitate entirely. This may be performed following centrifugation and sampling of the solution phase, in which case insolubility can be measured as a drop in solution protein concentration over time will be observed following centrifugation.

In an alternate embodiment, the oligomerization state is determined by monitoring relative mobility on native gel electrophoresis.

In another embodiment, the amount of protein that is expressed solubly in a prokaryotic host is determined. While factors other than the solubility of the native protein can impact levels of soluble expression, improvements in soluble expression may correlate with improvements in solubility. Any of a number of methods may be used; for example, following expression, SDS-polyacrylamide gel electrophoresis and/or western blots can be done on the soluble fraction of crude cell lysates or the expression media. There are also high throughput screens for soluble expression. In one embodiment, the protein of interest is fused to a fluorescent protein such as GFP, and the cells monitored for fluorescence (Waldo et. al. Nat. Biotechnol. 17: 691 (1999)). In an alternate embodiment, the protein of interest is fused to the antibiotic resistance enzyme chloramphenicol transferase. If the protein expresses solubly, the enzyme will be functional, thereby allowing growth on media with increased concentration of the antibiotic chloramphenicol (Maxwell et. al. Protein Sci. 8: 1908 (1999)). In another embodiment, the protein of interest is expressed as a fusion with the alpha domain of the enzyme beta-galactosidase. If the protein expresses in soluble form, the alpha domain will complement the omega domain to yield a functional enzyme. This may be detected as blue rather than white colony formation when the cells are plated on media containing the indicator X-gal (Wigley et. al. Nat. Biotechnol. 19: 131 (2001)).

Assaying the Activity of the Variants

In a preferred embodiment, the wild-type and variant proteins are analyzed for biological activities by suitable methods known in the art. Such assays include but are not limited to activation of interferon-responsive genes, receptor binding assays, antiviral activity assays, cytopathic effect inhibition assays, antiproliferative assays, immunomodulatory assays, and assays that monitor the induction of MHC molecules, all described in Meager, J. Immunol. Meth., 261:21-36 (2002).

In a preferred embodiment, wild type and variant proteins will be analyzed for their ability to activate interferon-sensitive signal transduction pathways. One example is the interferon-stimulated response element (ISRE) assay, described below and in the Examples. Cells which constitutively express the type I interferon receptor are transiently transfected with an ISRE-luciferase vector. After transfection, the cells are treated with an interferon variant. In a preferred embodiment, a number of protein concentrations, for example from 0.0001-10 ng/mL, are tested to generate a dose-response curve. In an alternate embodiment, two or more concentrations are tested. If the variant binds and activates its receptor, the resulting signal transduction cascade induces luciferase expression. Luminescence can be measured in a number of ways, for example by using a TopCount™ or Fusion microplate reader.

In a preferred embodiment, wild type and variant proteins will be analyzed for their ability to bind to the type I interferon receptor (IFNAR). Suitable binding assays include, but are not limited to, BIAcore assays (Pearce et al., Biochemistry 38:81-89 (1999)) and AlphaScreen™ assays (commercially available from PerkinElmer) (Bosse R., lily C., and Chelsky D (2002). Principles of AlphaScreen™ PerkinElmer Literature Application Note Ref# s4069. AlphaScreen™ is a bead-based non-radioactive luminescent proximity assay where the donor beads are excited by a laser at 680 nm to release singlet oxygen. The singlet oxygen diffuses and reacts with the thioxene derivative on the surface of acceptor beads leading to fluorescence emission at ˜600 nm. The fluorescence emission occurs only when the donor and acceptor beads are brought into close proximity by molecular interactions occurring when each is linked to ligand and receptor respectively. This ligand-receptor interaction can be competed away using receptor-binding variants while non-binding variants will not compete.

In an alternate preferred embodiment, wild type and variant proteins will be analyzed for their efficacy in treating an animal model of disease, such as the mouse or rat EAE model for multiple sclerosis.

Determining the Immunogenicity of the Variants

In a preferred embodiment, the immunogenicity of the IFN variants is determined experimentally to test whether the variant interferon proteins have reduced or eliminated immunogenicity relative to the wild type protein.

Increased protein solubility may decrease immunogenicity by reducing uptake by antigen presenting cells. Accordingly, in a preferred embodiment, uptake of wild type and variant interferon proteins by professional antigen presenting cells is monitored.

In a preferred embodiment, ex vivo T-cell activation assays are used to experimentally quantitate immunogenicity. In this method, antigen presenting cells and naïve T-cells from matched donors are challenged with a peptide or whole protein of interest one or more times. Then, T-cell activation can be detected using a number of methods, for example by monitoring production of cytokines or measuring uptake of tritiated thymidine. In the most preferred embodiment, interferon gamma production is monitored using Elispot assays (see Schmittel et. al. J. Immunol. Meth., 24: 17-24 (2000)).

In an alternate preferred embodiment, immunogenicity is measured in transgenic mouse systems. For example, mice expressing fully or partially human class II MHC molecules may be used.

In an alternate embodiment, immunogenicity is tested by administering the IFN variants to one or more animals, including rodents and primates, and monitoring for antibody formation.

Administration and Treatment Using IFN Variants

Once made, the variant IFN proteins and nucleic acids of the invention find use in a number of applications. In a preferred embodiment, a variant IFN protein or nucleic acid is administered to a patient to treat an IFN related disorder.

The administration of the variant IFN proteins of the present invention, preferably in the form of a sterile aqueous solution, may be done in a variety of ways, including, but not limited to, orally, parenterally, subcutaneously, intravenously, intranasally, transdermally, intraperitoneally, intramuscularly, intrapulmonary, vaginally, rectally, intranasally or intraocularly. In some instances, the variant IFN protein may be directly applied as a solution or spray. Depending upon the manner of introduction, the pharmaceutical composition may be formulated in a variety of ways.

The pharmaceutical compositions of the present invention comprise a variant IFN protein in a form suitable for administration to a patient. In the preferred embodiment, the pharmaceutical compositions are in a water-soluble form, such as being present as pharmaceutically acceptable salts, which is meant to include both acid and base addition salts.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the variant IFN proteins are added in a micellular formulation; see U.S. Pat. No. 5,833,948.

Combinations of pharmaceutical compositions may be administered. Moreover, the compositions may be administered in combination with other therapeutics.

In a preferred embodiment, the nucleic acid encoding the variant IFN proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. The oligonucleotides may be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11:205-210 (1993)). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

While the foregoing invention has been described above, it will be clear to one skilled in the art that various changes and additional embodiments made be made without departing from the scope of the invention. All publications, patents, patent applications (provisional, utility and PCT) or other documents cited herein are incorporated by references in their entirety.

### EXAMPLES

### Example 1

### Construction of a Homology Model of Interferon Kappa

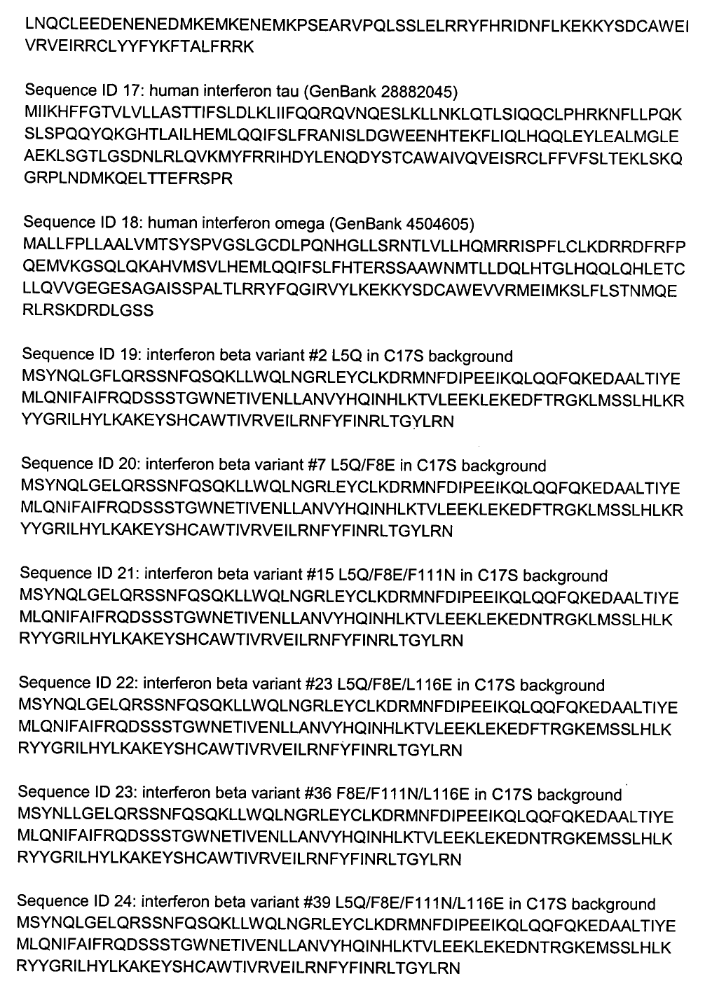

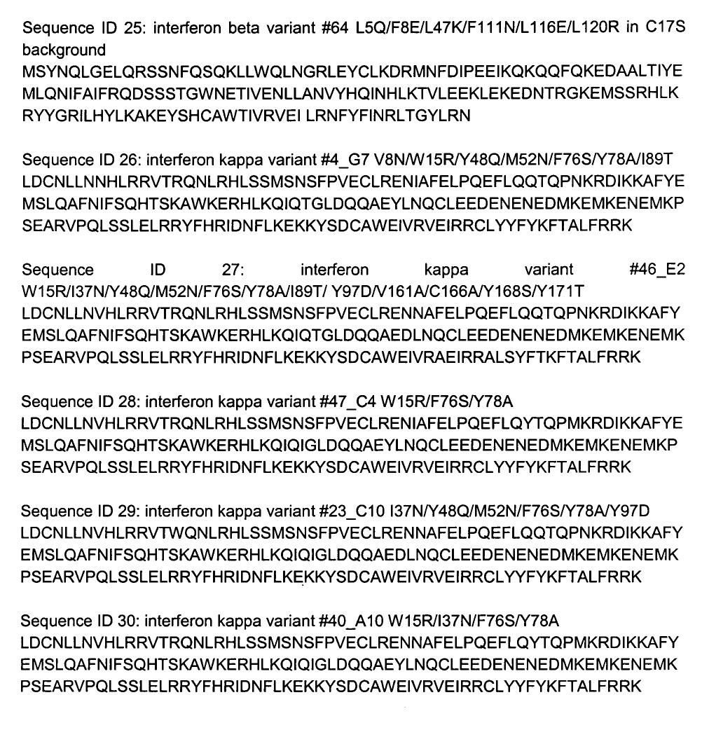

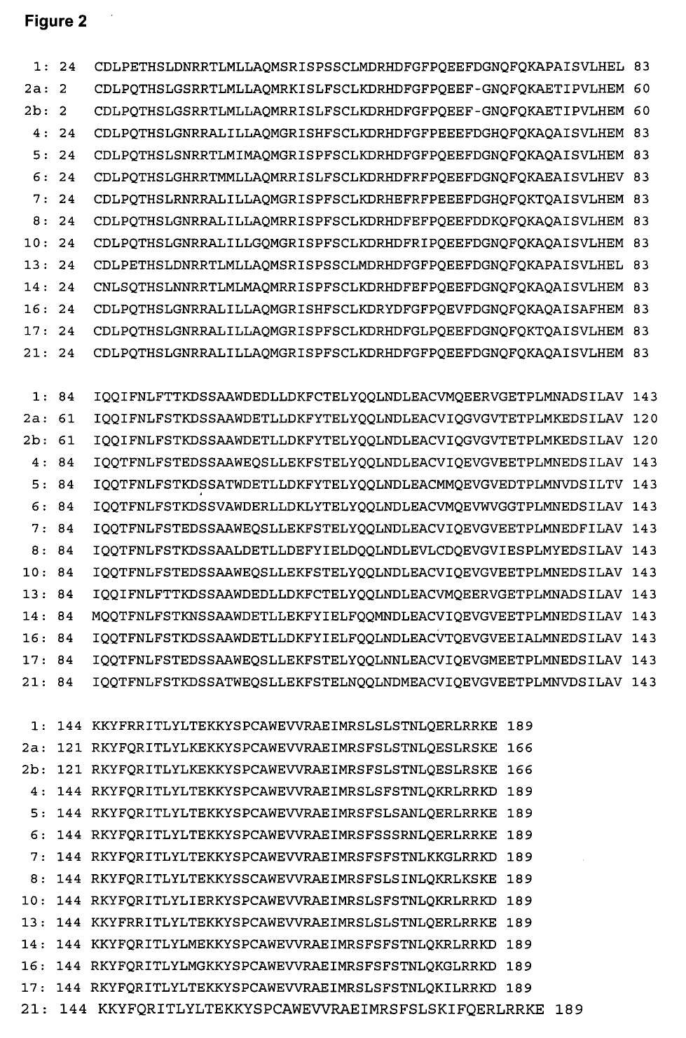

A homology model of interferon kappa was constructed based on the sequence of human interferon kappa (GenBank code 14488028), the crystal structures for interferon tau (PDB code 1BL5) and interferon beta (PDB code 1AU1), as well as the NMR structure for interferon alpha-2a (PDB code 1ITF). The sequences for interferons alpha-2a, beta, kappa, and tau were aligned using the multiple sequence alignment tool in the Homology model of the InsightII software package (Accelrys), as shown in FIG. 2. As the sequences share only approximately 35% identity, slightly different sequence alignments could have been used instead (see for example LaFleur et. al. J. Biol. Chem. 276: 39765-39771 (2001)). Based on similarity to the other interferon sequences, disulfide bonds are expected to be formed between residues C3 and C102 and between residues C32 and C 55 (LaFleur supra); these disufides were used as constraints in the generation of the homology models. A total of nine homology models were generated using the Modeler tool in the InsightII software package (Accelrys). The structures were analyzed for quality and the top four models were used in the analysis and design calculations described below.

### Example 2

### Identification of Exposed Hydrophobic Residues in Type I Interferons

A number of type I interferon structures were analyzed to identify solvent-exposed hydrophobic residues. The absolute and fractional solvent-exposed hydrophobic surface area of each residue was calculated using the method of Lee and Richards (J. Mol. Biol. 55: 379-400 (1971)) using an add-on radius of 1.4 Å (Angstroms). Each residue was also classified as core, boundary, or surface (see Dahiyat and Mayo Science 278: 82-87 (1997)).

Solvent exposed hydrophobic residues in interferon-alpha 2a were defined to be hydrophobic residues with at least 75 Å² (square Angstroms) exposed hydrophobic surface area in the interferon alpha-2a NMR structure (PDB code 1ITF, first molecule).

TABLE 1
Exposed hydrophobic residues in interferon-alpha 2a.

| | | core/ | exposed | percent |
|---|---|---|---|---|
| | | boundary/ | hydrophobic | hydrophobic |
| residue | # | surface | surface area | area exposed |
| MET | 16 | surface | 93.90 | 44.50 |
| PHE | 27 | surface | 172.10 | 69.10 |
| LEU | 30 | surface | 84.20 | 39.40 |
| TYR | 89 | surface | 80.00 | 41.10 |
| ILE | 100 | surface | 103.60 | 50.00 |
| LEU | 110 | surface | 151.30 | 70.20 |
| MET | 111 | surface | 76.40 | 35.60 |
| LEU | 117 | surface | 78.60 | 37.80 |
| LEU | 128 | surface | 104.30 | 50.40 |
| LEU | 161 | surface | 90.10 | 45.30 |

Solvent exposed hydrophobic residues in interferon beta were defined to be hydrophobic residues with at least 75 Å² (square Angstroms) exposed hydrophobic surface area in the interferon-beta crystal structure (PDB code 1AU1, chain A)

TABLE 2
Exposed hydrophobic residues in interferon-beta.

| | | core/ | exposed | percent |
|---|---|---|---|---|
| | | surface/ | hydrophobic | hydrophobic |
| residue | # | boundary | surface area | area buried |
| LEU | 5 | boundary | 100.30 | 48.30 |
| PHE | 8 | surface | 131.00 | 54.90 |
| PHE | 15 | surface | 151.90 | 63.30 |
| TRP | 22 | surface | 147.90 | 58.30 |
| LEU | 28 | boundary | 61.90 | 31.00 |
| TYR | 30 | surface | 129.00 | 66.80 |
| LEU | 32 | surface | 50.40 | 23.70 |
| MET | 36 | boundary | 82.60 | 40.00 |
| LEU | 47 | boundary | 72.20 | 35.50 |
| TYR | 92 | surface | 84.60 | 44.40 |
| PHE | 111 | surface | 196.30 | 80.10 |
| LEU | 116 | surface | 94.60 | 45.70 |
| LEU | 120 | surface | 67.20 | 32.50 |
| LEU | 130 | surface | 57.10 | 27.40 |
| VAL | 148 | boundary | 77.40 | 42.80 |
| TYR | 155 | surface | 88.60 | 46.30 |

Solvent exposed hydrophobic residues in interferon-kappa were defined to be hydrophobic residues with at least 30 Å² (square Angstroms) exposed hydrophobic surface area in at least one of the top four homology models (see above) and which were classified as boundary (B) or surface (S) in at least 3 of the 4 top structures. Solvent exposed hydrophobic residues in interferon kappa, along with their exposed hydrophobic surface area and C/S/B classification, are shown below.

TABLE 3
Exposed hydrophobic residues in interferon kappa.
Solvent exposed hydrophobic surface areas in square
Angstroms are given for the top four homology models.
Core/surface/boundary classification
is indicated as “C”, “S”, or “B” below.

| | | | |
|---|---|---|---|
| model 1 | model 2 | model 3 | model 4 |

| | | | | | | | | | |
|---|---|---|---|---|---|---|---|---|---|
| LEU | 1 | 134.57 | S | 135.88 | B | 91.03 | B | 134.11 | S |
| LEU | 5 | 102.62 | S | 89.78 | B | 70.67 | S | 103.39 | S |
| VAL | 8 | 70.36 | S | 76.97 | S | 70.19 | S | 72.51 | S |
| TRP | 15 | 155.63 | S | 161.08 | S | 149.83 | S | 153.22 | S |
| LEU | 18 | 33.86 | B | 42.72 | B | 64.82 | B | 34.39 | B |
| PHE | 28 | 39.03 | S | 32.47 | B | 16.19 | B | 34.43 | S |
| VAL | 30 | 118.49 | S | 112.38 | S | 43.12 | S | 118.23 | S |
| LEU | 33 | 92.00 | S | 73.35 | S | 72.73 | S | 93.60 | S |
| ILE | 37 | 106.52 | B | 127.16 | B | 99.30 | B | 106.28 | B |
| LEU | 46 | 84.43 | S | 86.04 | S | 84.47 | S | 83.90 | S |
| TYR | 48 | 79.98 | B | 60.73 | B | 93.88 | B | 81.91 | B |
| MET | 52 | 101.62 | B | 149.86 | S | 149.37 | S | 104.68 | S |
| LEU | 65 | 109.14 | B | 98.21 | S | 111.58 | B | 91.38 | S |
| PHE | 68 | 55.88 | B | 107.51 | B | 104.30 | B | 57.45 | B |
| PHE | 76 | 61.69 | B | 66.90 | B | 53.90 | B | 59.28 | B |
| TYR | 78 | 104.70 | B | 112.65 | S | 135.51 | B | 111.51 | B |
| TRP | 79 | 57.96 | S | 138.78 | B | 133.03 | C | 58.32 | S |
| ILE | 88 | 104.67 | S | 77.94 | S | 77.75 | S | 111.79 | S |
| TYR | 96 | 98.61 | B | 118.35 | B | 63.52 | B | 97.46 | B |
| MET | 111 | 118.98 | B | 152.74 | S | 115.40 | B | 109.32 | B |
| MET | 114 | 141.73 | S | 188.48 | S | 174.59 | S | 134.99 | B |
| MET | 119 | 147.52 | S | 173.09 | S | 159.56 | S | 134.72 | S |
| VAL | 126 | 23.49 | C | 77.29 | S | 70.45 | B | 54.01 | S |
| LEU | 132 | 86.27 | S | 95.70 | S | 81.83 | S | 84.16 | S |
| TYR | 150 | 41.55 | B | 62.57 | B | 86.01 | B | 45.22 | B |
| VAL | 160 | 49.02 | B | 69.23 | S | 70.61 | B | 49.02 | B |
| TYR | 167 | 99.52 | S | 84.23 | S | 149.46 | S | 100.52 | S |
| TYR | 170 | 63.85 | S | 77.37 | S | 110.88 | S | 61.83 | S |

The results in Table 3 were combined with the sequence analysis described in Example 4 to identify exposed hydrophobic residues in interferon kappa that could be replaced with polar residues without compromising the structure or function of the resulting variant protein.

Solvent exposed hydrophobic residues in ovine interferon tau were defined to be hydrophobic residues that were at least 25% exposed to solvent in the crystal structure of interferon tau (PDB code 1B5L).

TABLE 4
Exposed hydrophobic residues in interferon-tau.
The exposed hydrophobic surface areas

| | | | | Percent |
|---|---|---|---|---|
| | | C/S/B | Exposed | hydrophobic area |
| Residue | # | classification | hydrophobic area | burial |
| TYR | 2 | surface | 153.9 | 22.9 |
| LEU | 9 | surface | 85.8 | 59.1 |
| LEU | 24 | boundary | 121.1 | 42.5 |
| LEU | 30 | surface | 152.2 | 25.8 |
| TYR | 69 | surface | 71.6 | 62.5 |
| TRP | 77 | surface | 233.3 | 6.3 |
| MET | 114 | surface | 137.6 | 36.9 |
| VAL | 118 | surface | 103.9 | 42.9 |
| TYR | 136 | boundary | 53.3 | 72.6 |
| VAL | 146 | boundary | 64.5 | 63.9 |

### Example 3

### Identification of Dimer Interface Residues in Type I Interferons

Potential sites of interactions between interferon monomers were identified by examining contacts between monomers in the crystal structures of interferon molecules.

Interferon alpha-2b crystallized as a trimer of dimers (PDB code 1RH2), in which the dimer interface is zinc-mediated (see Radhakrishnan et. al. Structure 4: 1453-1463 (1996)). The zinc-mediated dimer is referred to herein as the “AB dimer”, while the interface between AB dimers is referred to as the “BC” dimer interface. The zinc-binding site comprises the residues Glu 41 and Glu 42. Additional residues that have been implicated in stabilizing the AB dimer interface include Lys 121, Asp 114, Gly 44, and Arg 33 (Radhakrishnan, supra).

Next, distance measurements were used to identify additional residues that may participate in intermolecular interactions. Residues that are within 8 Å (Angstroms) of the AB dimer interface (as measured by CA-CA distances) include: 35-37, 39-41, 44-46, 114-115, 117-118, 121-122, and 125. Residues that are within 8 Å of the BC dimer-dimer interface (as measured by CA-CA distances) include: 16, 19, 20, 25, 27, 28, 30, 33, 54, 58, 61, 65, 68, 85, 93, 99, 112, 113, and 149.

Interferon beta crystallized as an asymmetric dimer (PDB code 1AU1). Residues that are within 5 Å of the dimer interface (minimum heavy atom-heavy atom distance) include 42, 43, 46-49, 51, 113, 116, 117, 120, 121, and 124 (on chain A), as well as 1-6, 8, 9, 12, 16, 93, 96, 97, 100, 101, and 104 (on chain B).

### Example 4

### Identification of Residues Observed at Each Position in the Interferon Family

A large number of type I interferon sequences are known to exist, comprising interferons of different subtypes (e.g. alpha-2, alpha-4, beta, kappa), allelic variants (e.g. alpha-2a vs. alpha-2b), and interferons from different species. Analysis of these different interferon sequences can suggest substitutions that will be compatible with maintaining the structure and function of type I interferons.

The BLAST sequence alignment program was used to identify the 100 protein sequences in the nonredundant protein sequence database that are most closely related to interferon kappa. The annotations for these sequences were analyzed to confirm that all of the sequences are type one interferons. Next, the number of occurrences of each residue (and of deletions, denoted “-”) at each position in interferon kappa was determined. For example, the frequency of each residue at the exposed hydrophobic positions in interferon kappa is shown below.

TABLE 5
Frequency of each residue at exposed hydrophobic positions in interferon kappa.

| # | wt | — | A | C | D | E | F | G | H | I | K | L | M | N | P | Q | R | S | T | V | W | Y |
|---|---|---|---|---|---|---|---|---|---|---|---|---|---|---|---|---|---|---|---|---|---|---|
| 1 | L | 0 | 0 | 0 | 0 | 0 | 0 | 0 | 0 | 0 | 0 | 39 | 0 | 0 | 0 | 0 | 0 | 0 | 0 | 0 | 0 | 0 |
| 5 | L | 0 | 0 | 0 | 0 | 0 | 0 | 0 | 0 | 0 | 0 | 39 | 0 | 0 | 0 | 0 | 0 | 0 | 0 | 0 | 0 | 0 |
| 8 | V | 0 | 0 | 0 | 5 | 0 | 0 | 0 | 1 | 1 | 0 | 0 | 0 | 15 | 0 | 0 | 0 | 2 | 12 | 3 | 0 | 0 |
| 15 | W | 17 | 0 | 0 | 0 | 0 | 0 | 0 | 0 | 0 | 0 | 5 | 2 | 0 | 0 | 0 | 8 | 1 | 2 | 0 | 4 | 0 |
| 18 | L | 0 | 0 | 0 | 0 | 0 | 1 | 0 | 0 | 0 | 0 | 73 | 0 | 0 | 0 | 0 | 0 | 0 | 0 | 0 | 0 | 0 |
| 28 | F | 10 | 0 | 0 | 0 | 0 | 3 | 0 | 0 | 0 | 0 | 0 | 0 | 0 | 1 | 0 | 0 | 70 | 0 | 0 | 0 | 0 |
| 30 | V | 0 | 1 | 0 | 0 | 0 | 16 | 0 | 44 | 0 | 0 | 0 | 0 | 0 | 2 | 0 | 4 | 9 | 0 | 8 | 0 | 0 |
| 33 | L | 0 | 0 | 0 | 0 | 0 | 0 | 0 | 0 | 0 | 0 | 88 | 0 | 0 | 0 | 2 | 0 | 0 | 0 | 0 | 0 | 0 |
| 37 | I | 0 | 0 | 0 | 0 | 0 | 0 | 0 | 4 | 3 | 51 | 0 | 5 | 12 | 1 | 0 | 12 | 0 | 1 | 0 | 0 | 1 |
| 46 | L | 0 | 1 | 0 | 0 | 0 | 12 | 0 | 0 | 0 | 6 | 13 | 0 | 0 | 0 | 0 | 0 | 0 | 0 | 58 | 0 | 0 |
| 48 | Y | 0 | 0 | 0 | 1 | 0 | 0 | 78 | 0 | 0 | 0 | 5 | 0 | 0 | 1 | 1 | 0 | 0 | 0 | 1 | 0 | 3 |
| 52 | M | 1 | 0 | 0 | 0 | 0 | 0 | 0 | 0 | 0 | 0 | 0 | 3 | 0 | 2 | 80 | 4 | 0 | 0 | 0 | 0 | 0 |
| 65 | L | 0 | 0 | 0 | 0 | 0 | 0 | 0 | 0 | 0 | 0 | 3 | 0 | 0 | 0 | 87 | 0 | 0 | 0 | 0 | 0 | 0 |
| 68 | F | 0 | 0 | 0 | 0 | 0 | 85 | 0 | 0 | 0 | 0 | 2 | 0 | 0 | 0 | 0 | 0 | 1 | 0 | 1 | 0 | 1 |
| 76 | F | 0 | 11 | 1 | 0 | 0 | 4 | 0 | 0 | 0 | 0 | 0 | 0 | 0 | 1 | 0 | 0 | 73 | 0 | 0 | 0 | 0 |
| 78 | Y | 0 | 68 | 0 | 0 | 0 | 0 | 0 | 0 | 0 | 0 | 0 | 1 | 0 | 0 | 0 | 0 | 5 | 9 | 4 | 0 | 3 |
| 79 | W | 0 | 0 | 0 | 0 | 0 | 0 | 0 | 0 | 0 | 0 | 0 | 0 | 0 | 0 | 0 | 0 | 0 | 0 | 0 | 90 | 0 |
| 89 | I | 0 | 12 | 0 | 2 | 0 | 0 | 0 | 0 | 4 | 0 | 0 | 0 | 1 | 0 | 0 | 0 | 3 | 68 | 0 | 0 | 0 |
| 97 | Y | 0 | 0 | 0 | 46 | 6 | 0 | 5 | 26 | 0 | 0 | 0 | 0 | 4 | 0 | 0 | 0 | 0 | 0 | 0 | 0 | 3 |
| 112 | M | 43 | 13 | 0 | 0 | 0 | 6 | 0 | 0 | 1 | 0 | 1 | 5 | 0 | 3 | 0 | 0 | 1 | 10 | 7 | 0 | 0 |
| 115 | M | 19 | 4 | 0 | 0 | 3 | 0 | 6 | 0 | 5 | 1 | 8 | 16 | 0 | 0 | 0 | 0 | 27 | 1 | 0 | 0 | 0 |
| 120 | M | 38 | 0 | 0 | 0 | 0 | 0 | 0 | 0 | 1 | 0 | 5 | 44 | 0 | 0 | 0 | 0 | 0 | 0 | 2 | 0 | 0 |
| 127 | V | 77 | 0 | 0 | 0 | 0 | 0 | 4 | 0 | 0 | 0 | 2 | 3 | 0 | 0 | 0 | 0 | 0 | 0 | 4 | 0 | 0 |
| 133 | L | 4 | 0 | 0 | 0 | 0 | 0 | 0 | 0 | 0 | 0 | 64 | 0 | 0 | 0 | 0 | 0 | 0 | 0 | 22 | 0 | 0 |
| 151 | Y | 0 | 0 | 0 | 1 | 0 | 0 | 0 | 13 | 0 | 0 | 0 | 0 | 0 | 0 | 0 | 0 | 0 | 0 | 0 | 0 | 76 |
| 161 | V | 0 | 21 | 0 | 0 | 0 | 0 | 0 | 0 | 0 | 0 | 3 | 17 | 0 | 0 | 0 | 0 | 0 | 0 | 49 | 0 | 0 |
| 168 | Y | | | | | | | | | | | | | | | | | 12 | | | | |
| 171 | Y | 0 | 0 | 0 | 0 | 0 | 0 | 0 | 0 | 0 | 0 | 0 | 0 | 6 | 0 | 0 | 0 | 0 | 12 | 0 | 0 | 3 |

The raw frequencies above were normalized using the method of Henikoff & Henikoff (J. Mol. Biol. 243: 547-578 (1994)). Numerical values are only included for cells in which the number of occurrences in the table above is greater than 0.

TABLE 6
Normalized frequency of each residue at exposed hydrophobic positions in interferon kappa.

| # | wt | — | A | C | D | E | F | G | H | I | K | L | M | N | P | Q | R | S | T | V | W | Y |
|---|---|---|---|---|---|---|---|---|---|---|---|---|---|---|---|---|---|---|---|---|---|---|
| 1 | L | — | — | — | — | — | — | — | — | — | — | 0.6 | — | — | — | — | — | — | — | — | — | — |
| 5 | L | — | — | — | — | — | — | — | — | — | — | 0.6 | — | — | — | — | — | — | — | — | — | — |
| 8 | V | — | — | — | 0 | — | — | — | 0 | 0 | — | — | — | 0.1 | — | — | — | 0.1 | 0.2 | 0.2 | — | — |
| 15 | W | — | — | — | — | — | — | — | — | — | — | 0.1 | 0.1 | — | — | — | 0 | 0 | 0.1 | — | 0.2 | — |
| 18 | L | — | — | — | — | — | 0 | — | — | — | — | 0.7 | — | — | — | — | — | — | — | — | — | — |
| 28 | F | — | — | — | — | — | 0.2 | — | — | — | — | — | — | — | 0 | — | — | 0.4 | — | — | — | — |
| 30 | V | — | 0 | — | — | — | 0.2 | — | 0.1 | — | — | — | — | — | 0 | — | 0 | 0.3 | — | 0.2 | — | — |
| 33 | L | — | — | — | — | — | — | — | — | — | — | 1 | — | — | — | 0 | — | — | — | — | — | — |
| 37 | I | — | — | — | — | — | — | — | 0.1 | 0.2 | 0.1 | — | 0.1 | 0.3 | 0 | — | 0.1 | — | 0 | — | — | 0 |
| 46 | L | — | 0 | — | — | — | 0.2 | — | — | — | 0.2 | 0.5 | — | — | — | — | — | — | — | 0.2 | — | — |
| 48 | Y | — | — | — | 0 | — | — | 0.6 | — | — | — | 0.1 | — | — | 0 | 0 | — | — | — | 0 | — | 0.2 |
| 52 | M | — | — | — | — | — | — | — | — | — | — | — | 0.2 | — | 0 | 0.8 | 0 | — | — | — | — | — |
| 65 | L | — | — | — | — | — | — | — | — | — | — | 0.2 | — | — | — | 0.8 | — | — | — | — | — | — |
| 68 | F | — | — | — | — | — | 0.9 | — | — | — | — | 0 | — | — | — | — | — | 0 | — | 0 | — | 0 |
| 76 | F | — | 0.2 | 0 | — | — | 0.2 | — | — | — | — | — | — | — | 0 | — | — | 0.5 | — | — | — | — |
| 78 | Y | — | 0.4 | — | — | — | — | — | — | — | — | — | 0 | — | — | — | — | 0 | 0.3 | 0.1 | — | 0.2 |
| 79 | W | — | — | — | — | — | — | — | — | — | — | — | — | — | — | — | — | — | — | — | 1 | — |
| 89 | I | — | 0.4 | — | 0.1 | — | — | — | — | 0.2 | — | — | — | 0 | — | — | — | 0 | 0.4 | — | — | — |
| 97 | Y | — | — | — | 0.4 | 0 | — | 0 | 0.3 | — | — | — | — | 0.1 | — | — | — | — | — | — | — | 0.2 |
| 112 | M | — | 0.2 | — | — | — | 0.2 | — | — | 0 | — | 0 | 0.2 | — | 0 | — | — | 0 | 0.1 | 0 | — | — |
| 115 | M | — | 0 | — | — | 0 | — | 0.2 | — | 0.1 | 0 | 0.2 | 0.3 | — | — | — | — | 0.1 | 0 | — | — | — |
| 120 | M | — | — | — | — | — | — | — | — | 0 | — | 0 | 0.3 | — | — | — | — | — | — | 0 | — | — |
| 127 | V | — | — | — | — | — | — | 0 | — | — | — | 0.1 | 0 | — | — | — | — | — | — | 0.2 | — | — |
| 133 | L | — | — | — | — | — | — | — | — | — | — | 0.9 | — | — | — | — | — | — | — | 0 | — | — |
| 151 | Y | — | — | — | 0 | — | — | — | 0.3 | — | — | — | — | — | — | — | — | — | — | — | — | 0.7 |
| 161 | V | — | 0.4 | — | — | — | — | — | — | — | — | 0 | 0.1 | — | — | — | — | — | — | 0.5 | — | — |
| 168 | Y | | | | | | | | | | | | | | | | | 0.4 | | | | |
| 171 | Y | — | — | — | — | — | — | — | — | — | — | — | — | 0.2 | — | — | — | — | 0.4 | — | — | 0.2 |

This sequence alignment data was used in conjunction with the PDA®) technology calculations described above to identify suitable residues for different variable positions. If hydrophobicity at a given position was found to be conserved among interferons (i.e. the frequency of polar residues at that position was zero or very low), the position was not considered further. At the remaining positions, PDA®) technology calculations were performed to aid in the identification of suitable polar replacements.

Exposed hydrophobic positions at which polar residues are observed with a normalized frequency of 0.1 or greater include:

TABLE 7
Exposed hydrophobic positions in interferon-kappa at
which polar residues are observed with a normalized
frequency of at least 0.1 in other interferon proteins.

| # | wt | — | A | D | E | G | H | K | N | Q | R | S | T |
|---|---|---|---|---|---|---|---|---|---|---|---|---|---|
| 8 | V | — | — | 0 | — | — | 0 | — | 0.1 | — | — | 0.1 | 0.2 |
| 15 | W | — | — | — | — | — | — | — | — | — | 0 | 0 | 0.1 |
| 28 | F | — | — | — | — | — | — | — | — | — | — | 0.4 | — |
| 30 | V | — | 0 | — | — | — | 0.1 | — | — | — | 0 | 0.3 | — |
| 37 | I | — | — | — | — | — | 0.1 | 0.1 | 0.3 | — | 0.1 | — | 0 |
| 46 | L | — | 0 | — | — | — | — | 0.2 | — | — | — | — | — |
| 48 | Y | — | — | 0 | — | 0.6 | — | — | — | 0 | — | — | — |
| 52 | M | — | — | — | — | — | — | — | — | 0.8 | 0 | — | — |
| 65 | L | — | — | — | — | — | — | — | — | 0.8 | — | — | — |
| 76 | F | — | 0.2 | — | — | — | — | — | — | — | — | 0.5 | — |
| 78 | Y | — | 0.4 | — | — | — | — | — | — | — | — | 0 | 0.3 |
| 89 | I | — | 0.4 | 0.1 | — | — | — | — | 0 | — | — | 0 | 0.4 |
| 97 | Y | — | — | 0.4 | 0 | 0 | 0.3 | — | 0.1 | — | — | — | — |
| 112 | M | — | 0.2 | — | — | — | — | — | — | — | — | 0 | 0.1 |
| 115 | M | — | 0 | — | 0 | 0.2 | — | 0 | — | — | — | 0.1 | 0 |
| 151 | Y | — | — | 0 | — | — | 0.3 | — | — | — | — | — | — |
| 161 | V | — | 0.4 | — | — | — | — | — | — | — | — | — | — |
| 168 | Y | — | — | — | — | — | — | — | — | — | — | 0.4 | — |
| 171 | Y | — | — | — | — | — | — | — | 0.2 | — | — | — | 0.4 |

### EXAMPLE 5

### Identification of Suitable Replacements for Exposed Hydrophobic Residues

PDA® technology calculations were performed to identify polar residues that are compatible with the structure and function of type I interferons. Energies were calculated for alanine and each of the polar residues at each exposed hydrophobic position, using a force field describing van der Waals interactions (VDW), electrostatics (Elec), hydrogen bonds (Hbond), and solvation (Solv). The energy of the wild type hydrophobic residue was also calculated. Polar residues with total energies that were similar to or more favorable than the wild type hydrophobic residue (the first line below for each position) were considered to be compatible with the target interferon (* below), and the polar residues with the most favorable energies were especially preferred (** below). Histidine was modeled in two possible states: “HSP” is the doubly-protonated state of histidine, while “HIS” is neutral histidine.

TABLE 8
Interferon-alpha calculation results, exposed hydrophobic residues

| | # | AA | Total | VDW | Elec | HBond | Solv |
|---|---|---|---|---|---|---|---|
| | 16 | MET | 9.68 | −4.05 | 0.00 | 0.00 | 13.729 |
| * | 16 | ALA | 3.87 | −1.65 | 0.00 | 0.00 | 5.522 |
| ** | 16 | ASP | −1.33 | −2.85 | −0.40 | 0.00 | 1.9233 |
| * | 16 | GLU | 1.55 | −3.19 | −0.40 | 0.00 | 5.1371 |
| * | 16 | HIS | 3.90 | −3.60 | 0.00 | 0.00 | 7.4983 |
| * | 16 | HSP | 3.91 | −3.62 | 0.27 | 0.00 | 7.2511 |
| * | 16 | LYS | 5.22 | −3.31 | 0.31 | 0.00 | 8.2164 |
| * | 16 | ASN | 0.86 | −2.88 | 0.01 | 0.00 | 3.7346 |
| * | 16 | GLN | 0.70 | −3.20 | −0.04 | 0.00 | 3.9397 |
| * | 16 | ARG | 0.73 | −3.36 | 0.22 | 0.00 | 3.8702 |
| * | 16 | SER | 0.00 | −1.94 | 0.00 | 0.00 | 1.9394 |
| * | 16 | THR | 3.55 | −2.89 | 0.04 | 0.00 | 6.4007 |
| | 27 | PHE | 20.55 | −2.52 | 0.00 | 0.00 | 23.0764 |
| * | 27 | ALA | 6.99 | −0.82 | 0.00 | 0.00 | 7.8098 |
| * | 27 | ASP | 1.27 | −1.51 | −0.38 | 0.00 | 3.1569 |
| * | 27 | GLU | 1.76 | −1.53 | −0.22 | 0.00 | 3.5092 |
| * | 27 | HIS | 11.57 | −1.76 | −0.01 | 0.00 | 13.3424 |
| * | 27 | HSP | 11.16 | −1.76 | 0.16 | 0.00 | 12.7635 |
| * | 27 | LYS | 7.36 | −2.10 | 0.25 | 0.00 | 9.2138 |
| ** | 27 | ASN | 0.52 | −1.52 | −0.06 | 0.00 | 2.091 |
| ** | 27 | GLN | 0.89 | −1.54 | 0.00 | 0.00 | 2.4286 |
| * | 27 | ARG | 5.35 | −1.59 | 0.21 | 0.00 | 6.7299 |
| * | 27 | SER | 1.63 | −1.00 | −0.03 | 0.00 | 2.6514 |
| * | 27 | THR | 6.62 | −1.40 | −0.03 | 0.00 | 8.0523 |
| | 100 | ILE | 6.17 | −4.09 | 0.00 | 0.00 | 10.2668 |
| * | 100 | ALA | 3.44 | −1.47 | 0.00 | 0.00 | 4.9013 |
| * | 100 | ASP | −0.59 | −2.28 | 0.24 | 0.00 | 1.4537 |
| ** | 100 | GLU | −1.26 | −3.19 | 0.50 | 0.00 | 1.4374 |
| | 100 | HIS | 15.87 | 0.86 | −0.01 | 0.00 | 15.0219 |
| | 100 | HSP | 15.16 | 0.98 | −0.20 | 0.00 | 14.3823 |
| * | 100 | LYS | 1.23 | −3.37 | −0.38 | 0.00 | 4.9902 |
| * | 100 | ASN | 0.38 | −3.14 | 0.00 | 0.00 | 3.5252 |
| ** | 100 | GLN | −2.56 | −3.28 | 0.02 | 0.00 | 0.7041 |
| ** | 100 | ARG | −1.57 | −3.39 | −0.27 | 0.00 | 2.0909 |
| * | 100 | SER | −0.30 | −1.72 | −0.01 | 0.00 | 1.4346 |
| * | 100 | THR | 4.32 | −2.62 | 0.00 | 0.00 | 6.9432 |
| | 110 | LEU | 18.52 | −1.89 | 0.00 | 0.00 | 20.4107 |
| * | 110 | ALA | 8.94 | −0.77 | 0.00 | 0.00 | 9.7089 |
| * | 110 | ASP | 3.92 | −1.36 | 0.17 | 0.00 | 5.1126 |
| * | 110 | GLU | 4.44 | −2.34 | 0.61 | 0.00 | 6.1639 |
| * | 110 | HIS | 13.80 | −1.79 | 0.00 | 0.00 | 15.5913 |
| * | 110 | HSP | 13.11 | −1.79 | −0.10 | 0.00 | 15.0058 |
| * | 110 | LYS | 11.14 | −1.96 | −0.23 | 0.00 | 13.3274 |
| ** | 110 | ASN | 2.75 | −1.37 | −0.04 | 0.00 | 4.1649 |
| ** | 110 | GLN | 2.83 | −2.34 | 0.06 | 0.00 | 5.1235 |
| * | 110 | ARG | 6.17 | −0.09 | −0.23 | 0.00 | 6.4996 |
| ** | 110 | SER | 3.03 | −0.94 | −0.02 | 0.00 | 3.9872 |
| * | 110 | THR | 4.82 | −1.84 | −0.03 | 0.00 | 6.7023 |
| | 111 | MET | 1.37 | −4.94 | 0.00 | 0.00 | 6.308 |
| | 111 | ALA | 5.58 | −1.21 | 0.00 | 0.00 | 6.7846 |
| * | 111 | ASP | 0.88 | −2.06 | 0.41 | 0.00 | 2.534 |
| * | 111 | GLU | 0.33 | −2.52 | 0.42 | 0.00 | 2.4273 |
| | 111 | HIS | 2.55 | −3.90 | −0.01 | 0.00 | 6.4709 |
| | 111 | HSP | 3.57 | −3.92 | −1.10 | 0.00 | 8.5877 |
| | 111 | LYS | 2.18 | −2.62 | −0.28 | 0.00 | 5.0789 |
| * | 111 | ASN | 0.14 | −2.09 | 0.05 | 0.00 | 2.1808 |
| ** | 111 | GLN | −0.92 | −2.54 | −0.05 | 0.00 | 1.6617 |
| * | 111 | ARG | 1.21 | −2.71 | −0.44 | 0.00 | 4.3527 |
| * | 111 | SER | 1.29 | −1.46 | 0.02 | 0.00 | 2.7337 |
| ** | 111 | THR | −0.16 | −3.15 | 0.05 | 0.00 | 2.9415 |
| | 117 | LEU | 3.03 | −4.07 | 0.00 | 0.00 | 7.0989 |
| * | 117 | ALA | −1.03 | −1.74 | 0.00 | 0.00 | 0.7126 |
| ** | 117 | ASP | −3.58 | −3.54 | 0.63 | 0.00 | −0.6613 |
| ** | 117 | GLU | −3.35 | −3.35 | 0.26 | 0.00 | −0.2511 |
| | 117 | HIS | 3.54 | −3.46 | −0.08 | 0.00 | 7.0827 |
| | 117 | HSP | 3.69 | −3.26 | 0.46 | 0.00 | 6.5019 |
| * | 117 | LYS | −1.42 | −4.06 | −0.48 | 0.00 | 3.1122 |
| * | 117 | ASN | −0.83 | −3.24 | −0.11 | 0.00 | 2.5211 |
| ** | 117 | GLN | −4.34 | −3.37 | 0.06 | 0.00 | −1.0372 |
| ** | 117 | ARG | −3.91 | −1.54 | −0.49 | −2.87 | 0.9774 |
| ** | 117 | SER | −3.47 | −2.09 | −0.03 | 0.00 | −1.3545 |
| * | 117 | THR | −1.87 | −3.00 | −0.02 | 0.00 | 1.1538 |
| | 161 | LEU | 10.25 | −3.57 | 0.00 | 0.00 | 13.8222 |
| * | 161 | ALA | 2.72 | −1.25 | 0.00 | 0.00 | 3.9705 |
| * | 161 | ASP | −0.17 | −2.59 | −0.04 | −0.11 | 2.5728 |
| ** | 161 | GLU | −2.33 | −3.04 | 0.15 | 0.00 | 0.5566 |
| * | 161 | HIS | 2.94 | −4.91 | −0.03 | 0.00 | 7.8882 |
| * | 161 | HSP | 4.64 | −4.93 | −0.19 | 0.00 | 9.7575 |
| ** | 161 | LYS | −1.13 | −3.55 | −0.20 | 0.00 | 2.6196 |
| * | 161 | ASN | −0.29 | −2.17 | −0.07 | 0.00 | 1.943 |
| * | 161 | GLN | −0.66 | −3.07 | −0.03 | 0.00 | 2.4459 |
| * | 161 | ARG | −0.43 | −4.56 | −1.02 | −4.78 | 9.9354 |
| * | 161 | SER | 0.34 | −1.58 | −0.04 | 0.00 | 1.9577 |
| * | 161 | THR | 0.71 | −2.75 | −0.04 | 0.00 | 3.4958 |

TABLE 9
Interferon beta calculation results, exposed hydrophobic residues

| | # | AA | Total | VDW | Elec | HBond | Solv |
|---|---|---|---|---|---|---|---|
| | 5 | LEU | 6.86 | −4.43 | 0.00 | 0.00 | 11.28 |
| * | 5 | ALA | 1.42 | −1.74 | 0.00 | 0.00 | 3.16 |
| ** | 5 | ASP | −2.63 | −2.74 | −0.37 | 0.00 | 0.47 |
| ** | 5 | GLU | −3.43 | −3.98 | −0.31 | 0.00 | 0.87 |
| | 5 | HIS | 13.88 | −0.11 | −0.09 | 0.00 | 14.07 |
| | 5 | HSP | 13.62 | −0.01 | 0.08 | 0.00 | 13.55 |
| * | 5 | LYS | −0.35 | −4.39 | 0.18 | 0.00 | 3.86 |
| * | 5 | ASN | −0.15 | −2.77 | 0.02 | 0.00 | 2.61 |
| ** | 5 | GLN | −3.95 | −4.00 | −0.03 | 0.00 | 0.08 |
| * | 5 | ARG | 0.17 | −3.17 | 0.21 | 0.00 | 3.12 |
| ** | 5 | SER | −3.45 | −2.03 | −0.02 | 0.00 | −1.40 |
| ** | 5 | THR | −2.86 | −3.43 | −0.02 | 0.00 | 0.59 |
| | 8 | PHE | 11.34 | −4.41 | 0.00 | 0.00 | 15.75 |
| * | 8 | ALA | −0.23 | −1.77 | 0.00 | 0.00 | 1.54 |
| ** | 8 | ASP | −3.43 | −2.73 | −0.34 | 0.00 | −0.37 |
| ** | 8 | GLU | −2.58 | −4.05 | −0.30 | 0.00 | 1.77 |
| * | 8 | HIS | 6.12 | −3.53 | 0.08 | 0.00 | 9.57 |
| * | 8 | HSP | 6.14 | −3.54 | 0.47 | 0.00 | 9.20 |
| * | 8 | LYS | 2.74 | −3.94 | 0.24 | 0.00 | 6.44 |
| * | 8 | ASN | −1.13 | −2.74 | −0.02 | 0.00 | 1.63 |
| ** | 8 | GLN | −2.86 | −2.46 | −0.08 | −2.76 | 2.44 |
| * | 8 | ARG | −1.50 | −4.00 | 0.33 | 0.00 | 2.17 |
| ** | 8 | SER | −4.37 | −2.02 | −0.02 | 0.00 | −2.33 |
| * | 8 | THR | 3.32 | −3.02 | −0.08 | 0.00 | 6.42 |
| | 15 | PHE | 16.43 | −3.32 | 0.00 | 0.00 | 19.75 |
| * | 15 | ALA | 4.13 | −1.43 | 0.00 | 0.00 | 5.55 |
| ** | 15 | ASP | −2.05 | −2.23 | −0.22 | 0.00 | 0.40 |
| * | 15 | GLU | −0.61 | −2.42 | −0.19 | 0.00 | 2.01 |
| * | 15 | HIS | 8.24 | −2.87 | −0.01 | 0.00 | 11.11 |
| * | 15 | HSP | 7.89 | −2.87 | 0.22 | 0.00 | 10.54 |
| * | 15 | LYS | 4.45 | −2.65 | 0.18 | 0.00 | 6.92 |
| * | 15 | ASN | −0.40 | −2.86 | 0.01 | 0.00 | 2.45 |
| ** | 15 | GLN | −1.29 | −2.45 | 0.01 | 0.00 | 1.15 |
| * | 15 | ARG | 0.02 | −2.55 | 0.20 | 0.00 | 2.36 |
| ** | 15 | SER | −1.36 | −1.64 | 0.00 | 0.00 | 0.27 |
| * | 15 | THR | 4.55 | −2.43 | 0.02 | 0.00 | 6.96 |
| | 22 | TRP | 18.45 | −5.92 | 0.00 | 0.00 | 24.37 |
| * | 22 | ALA | 4.20 | −1.41 | 0.00 | 0.00 | 5.61 |
| * | 22 | ASP | 0.36 | −2.04 | −0.31 | 0.00 | 2.71 |
| ** | 22 | GLU | −1.48 | −3.44 | −0.22 | 0.00 | 2.18 |
| * | 22 | HIS | 11.29 | 0.90 | −0.15 | 0.00 | 10.54 |
| * | 22 | HSP | 10.51 | 0.24 | −0.05 | 0.00 | 10.32 |
| * | 22 | LYS | 1.76 | −3.78 | 0.24 | 0.00 | 5.31 |
| * | 22 | ASN | 0.23 | −2.05 | −0.05 | 0.00 | 2.33 |
| ** | 22 | GLN | −2.43 | −3.44 | 0.01 | 0.00 | 1.00 |
| * | 22 | ARG | 0.66 | −3.42 | 0.23 | 0.00 | 3.84 |
| ** | 22 | SER | −1.24 | −1.58 | −0.01 | 0.00 | 0.35 |
| * | 22 | THR | 3.43 | −2.85 | 0.05 | 0.00 | 6.22 |
| | 28 | LEU | 2.83 | −5.56 | 0.00 | 0.00 | 8.40 |
| * | 28 | ALA | 2.61 | −1.61 | 0.00 | 0.00 | 4.21 |
| * | 28 | ASP | 1.55 | −3.49 | 0.01 | 0.00 | 5.03 |
| * | 28 | GLU | −1.66 | −3.82 | −0.04 | 0.00 | 2.20 |
| | 28 | HIS | 4.28 | −5.06 | 0.06 | 0.00 | 9.28 |
| | 28 | HSP | 5.23 | −4.96 | 0.04 | −0.73 | 10.88 |
| * | 28 | LYS | −0.87 | −4.43 | −0.01 | 0.00 | 3.57 |
| * | 28 | ASN | 0.72 | −3.46 | 0.04 | 0.00 | 4.14 |
| ** | 28 | GLN | −6.92 | −3.78 | −0.11 | −5.30 | 2.27 |
| | 28 | ARG | 3.10 | −6.28 | 0.21 | 0.00 | 9.17 |
| * | 28 | SER | 0.59 | −2.01 | −0.01 | 0.00 | 2.62 |
| | 28 | THR | 7.09 | −2.50 | 0.01 | 0.00 | 9.57 |
| | 30 | TYR | 13.74 | −3.59 | −0.05 | 0.00 | 17.38 |
| * | 30 | ALA | 10.72 | −0.88 | 0.00 | 0.00 | 11.60 |
| ** | 30 | ASP | 3.32 | −1.36 | −0.24 | 0.00 | 4.92 |
| * | 30 | GLU | 5.32 | −1.88 | −0.29 | 0.00 | 7.49 |
| * | 30 | HIS | 9.66 | −2.99 | −0.08 | 0.00 | 12.73 |
| * | 30 | HSP | 12.47 | −3.00 | 0.74 | 0.00 | 14.73 |
| * | 30 | LYS | 8.65 | −2.26 | 0.19 | 0.00 | 10.72 |
| ** | 30 | ASN | 2.78 | −1.37 | 0.01 | 0.00 | 4.15 |
| * | 30 | GLN | 4.45 | −1.89 | −0.01 | 0.00 | 6.35 |
| * | 30 | ARG | 7.17 | −1.90 | 0.15 | 0.00 | 8.93 |
| * | 30 | SER | 4.49 | −1.03 | −0.02 | 0.00 | 5.54 |
| * | 30 | THR | 7.17 | −1.69 | −0.02 | 0.00 | 8.88 |
| | 32 | LEU | 0.79 | −4.68 | 0.00 | 0.00 | 5.47 |
| ** | 32 | ALA | −0.14 | −1.52 | 0.00 | 0.00 | 1.38 |
| | 32 | ASP | 1.58 | −3.02 | −0.21 | 0.00 | 4.81 |
| * | 32 | GLU | 0.18 | −4.32 | −0.47 | 0.00 | 4.97 |
| * | 32 | HIS | −0.42 | −4.84 | −0.17 | 0.00 | 4.58 |
| ** | 32 | HSP | −0.93 | −4.84 | −0.22 | 0.00 | 4.13 |
| | 32 | LYS | 2.85 | −4.41 | 0.39 | 0.00 | 6.87 |
| | 32 | ASN | 3.94 | −3.09 | −0.04 | 0.00 | 7.06 |
| * | 32 | GLN | 0.22 | −4.00 | 0.01 | 0.00 | 4.21 |
| * | 32 | ARG | 0.95 | −4.74 | 0.36 | 0.00 | 5.33 |
| * | 32 | SER | 0.83 | −1.93 | 0.06 | 0.00 | 2.70 |
| | 32 | THR | 1.72 | −3.10 | 0.06 | 0.00 | 4.76 |
| | 36 | MET | 0.14 | −5.60 | 0.00 | 0.00 | 5.74 |
| | 36 | ALA | 0.38 | −1.86 | 0.00 | 0.00 | 2.24 |
| ** | 36 | ASP | −3.06 | −3.47 | 0.02 | −0.03 | 0.43 |
| ** | 36 | GLU | −3.53 | −3.34 | −0.05 | 0.00 | −0.14 |
| * | 36 | HIS | −0.84 | −5.33 | 0.03 | 0.00 | 4.46 |
| | 36 | HSP | 0.32 | −5.04 | −0.08 | 0.00 | 5.44 |
| ** | 36 | LYS | −3.76 | −4.99 | 0.00 | 0.00 | 1.22 |
| * | 36 | ASN | −1.09 | −3.53 | 0.00 | −0.05 | 2.48 |
| ** | 36 | GLN | −5.26 | −2.66 | −0.10 | −2.32 | −0.18 |
| * | 36 | ARG | −2.19 | −2.92 | 0.05 | 0.00 | 0.69 |
| * | 36 | SER | −2.41 | −2.27 | 0.02 | 0.00 | −0.17 |
| 2** | 36 | THR | −3.93 | −1.20 | 0.02 | 0.00 | −2.76 |
| | 47 | LEU | 1.86 | −6.08 | 0.00 | 0.00 | 7.94 |
| * | 47 | ALA | 0.52 | −2.11 | 0.00 | 0.00 | 2.62 |
| ** | 47 | ASP | −7.26 | −4.20 | −0.37 | −2.90 | 0.22 |
| * | 47 | GLU | −2.33 | −4.94 | 0.02 | 0.00 | 2.59 |
| | 47 | HIS | 217.36 | 213.11 | 0.09 | 0.00 | 4.16 |
| | 47 | HSP | 4313.02 | 4309.27 | −2.51 | 0.00 | 6.27 |
| ** | 47 | LYS | −5.22 | −5.97 | 0.01 | 0.00 | 0.74 |
| ** | 47 | ASN | −4.27 | −4.31 | −0.18 | −2.14 | 2.37 |
| * | 47 | GLN | −1.65 | −5.40 | −0.07 | −2.13 | 5.95 |
| * | 47 | ARG | −3.84 | −4.76 | −0.27 | −6.29 | 7.49 |
| * | 47 | SER | −1.23 | −2.64 | 0.03 | 0.00 | 1.37 |
| * | 47 | THR | −0.02 | −2.58 | 0.01 | 0.00 | 2.56 |
| | 92 | TYR | 3.84 | −5.11 | 0.01 | 0.00 | 8.95 |
| * | 92 | ALA | −1.94 | −1.95 | 0.00 | 0.00 | 0.01 |
| ** | 92 | ASP | −5.45 | −3.06 | −0.33 | −0.01 | −2.04 |
| ** | 92 | GLU | −5.14 | −3.67 | −0.08 | 0.00 | −1.40 |
| * | 92 | HIS | 3.04 | −4.25 | −0.04 | 0.00 | 7.33 |
| * | 92 | HSP | 2.94 | −4.25 | 0.28 | 0.00 | 6.91 |
| * | 92 | LYS | −1.75 | −3.96 | 0.00 | 0.00 | 2.21 |
| * | 92 | ASN | −3.30 | −3.13 | −0.12 | −0.03 | −0.02 |
| ** | 92 | GLN | −5.55 | −3.69 | 0.02 | 0.00 | −1.89 |
| * | 92 | ARG | −0.49 | −3.72 | 0.14 | 0.00 | 3.10 |
| ** | 92 | SER | −4.90 | −2.25 | −0.03 | 0.00 | −2.62 |
| | 92 | THR | 4.46 | 0.21 | 0.00 | 0.00 | 4.25 |
| | 111 | PHE | 29.59 | −2.42 | 0.00 | 0.00 | 32.01 |
| * | 111 | ALA | 15.98 | −0.76 | 0.00 | 0.00 | 16.74 |
| ** | 111 | ASP | 8.56 | −1.11 | 0.03 | 0.00 | 9.64 |
| * | 111 | GLU | 13.15 | −1.18 | −0.07 | 0.00 | 14.39 |
| * | 111 | HIS | 19.66 | −1.33 | 0.00 | 0.00 | 20.99 |
| * | 111 | HSP | 19.06 | −1.33 | −0.02 | 0.00 | 20.41 |
| * | 111 | LYS | 20.27 | −1.30 | 0.08 | 0.00 | 21.49 |
| ** | 111 | ASN | 7.32 | −1.10 | 0.00 | 0.00 | 8.41 |
| * | 111 | GLN | 11.91 | −1.18 | −0.03 | 0.00 | 13.12 |
| * | 111 | ARG | 15.55 | −1.25 | 0.02 | 0.00 | 16.78 |
| ** | 111 | SER | 9.49 | −0.86 | 0.01 | 0.00 | 10.34 |
| * | 111 | THR | 14.87 | −0.10 | −0.10 | −0.71 | 15.78 |
| | 116 | LEU | 4.71 | −3.66 | 0.00 | 0.00 | 8.37 |
| * | 116 | ALA | 1.74 | −1.32 | 0.00 | 0.00 | 3.06 |
| ** | 116 | ASP | −2.58 | −2.25 | −0.19 | 0.00 | −0.13 |
| * | 116 | GLU | −1.53 | −3.11 | −0.11 | 0.00 | 1.69 |
| | 116 | HIS | 7.67 | −3.22 | 0.11 | 0.00 | 10.78 |
| | 116 | HSP | 7.44 | −3.22 | 0.50 | 0.00 | 10.16 |
| * | 116 | LYS | 1.45 | −3.27 | 0.03 | 0.00 | 4.68 |
| ** | 116 | ASN | −2.54 | −2.29 | −0.05 | 0.00 | −0.20 |
| * | 116 | GLN | −1.95 | −3.13 | −0.01 | 0.00 | 1.18 |
| * | 116 | ARG | −1.05 | −3.53 | 0.29 | 0.00 | 2.18 |
| * | 116 | SER | −1.66 | −1.55 | −0.01 | 0.00 | −0.10 |
| * | 116 | THR | 1.59 | −1.87 | −0.01 | 0.00 | 3.47 |
| | 120 | LEU | 0.81 | −6.47 | 0.00 | 0.00 | 7.28 |
| | 120 | ALA | 2.03 | −1.44 | 0.00 | 0.00 | 3.46 |
| ** | 120 | ASP | −2.85 | −2.28 | −0.33 | 0.00 | −0.24 |
| | 120 | GLU | 1.19 | −2.64 | −0.16 | 0.00 | 3.99 |
| | 120 | HIS | 10.00 | −3.07 | 0.08 | 0.00 | 12.99 |
| | 120 | HSP | 9.96 | −2.91 | 0.20 | 0.00 | 12.68 |
| | 120 | LYS | 6.44 | −2.73 | 0.30 | 0.00 | 8.87 |
| * | 120 | ASN | −1.33 | −2.21 | −0.05 | 0.00 | 0.94 |
| * | 120 | GLN | 0.39 | −2.66 | 0.04 | 0.00 | 3.01 |
| | 120 | ARG | 4.28 | −2.64 | 0.23 | 0.00 | 6.69 |
| ** | 120 | SER | −2.59 | −1.64 | −0.05 | 0.00 | −0.90 |
| | 120 | THR | 3.04 | −3.74 | −0.01 | 0.00 | 6.80 |
| | 130 | LEU | −4.92 | −5.89 | 0.00 | 0.00 | 0.98 |
| | 130 | ALA | 0.46 | −1.57 | 0.00 | 0.00 | 2.03 |
| * | 130 | ASP | −4.43 | −2.75 | −0.13 | 0.00 | −1.55 |
| ** | 130 | GLU | −6.43 | −3.00 | −0.16 | 0.00 | −3.28 |
| | 130 | HIS | 0.41 | −4.27 | −0.03 | 0.00 | 4.71 |
| | 130 | HSP | 2.99 | −4.38 | 0.03 | 0.00 | 7.34 |
| * | 130 | LYS | −4.72 | −5.08 | 0.18 | 0.00 | 0.19 |
| * | 130 | ASN | −4.59 | −2.79 | 0.00 | 0.00 | −1.80 |
| ** | 130 | GLN | −6.62 | −4.38 | 0.01 | 0.00 | −2.25 |
| ** | 130 | ARG | −5.87 | −5.87 | −0.01 | −2.32 | 2.33 |
| | 130 | SER | −3.50 | −1.84 | 0.00 | 0.00 | −1.66 |
| | 130 | THR | −3.29 | −3.41 | 0.02 | 0.00 | 0.09 |
| | 148 | VAL | 6.65 | −3.33 | 0.00 | 0.00 | 9.98 |
| | 148 | ALA | 7.09 | −1.45 | 0.00 | 0.00 | 8.54 |
| ** | 148 | ASP | 0.64 | −2.35 | −0.29 | 0.00 | 3.28 |
| ** | 148 | GLU | 1.02 | −3.73 | −0.30 | 0.00 | 5.06 |
| | 148 | HIS | 7.65 | −3.09 | −0.04 | 0.00 | 10.79 |
| | 148 | HSP | 7.26 | −3.10 | 0.16 | 0.00 | 10.20 |
| * | 148 | LYS | 2.96 | −4.18 | 0.36 | 0.00 | 6.77 |
| * | 148 | ASN | 2.53 | −2.37 | −0.02 | 0.00 | 4.92 |
| * | 148 | GLN | 2.96 | −2.72 | 0.03 | 0.00 | 5.64 |
| ** | 148 | ARG | 1.86 | −3.88 | 0.34 | 0.00 | 5.40 |
| ** | 148 | SER | 1.08 | −1.68 | 0.00 | 0.00 | 2.77 |
| * | 148 | THR | 5.24 | −2.58 | 0.03 | 0.00 | 7.79 |
| | 155 | TYR | 6.95 | −4.80 | −0.01 | 0.00 | 11.76 |
| * | 155 | ALA | 4.11 | −1.52 | 0.00 | 0.00 | 5.63 |
| ** | 155 | ASP | −1.98 | −2.45 | −0.29 | 0.00 | 0.76 |
| * | 155 | GLU | −0.57 | −3.62 | −0.27 | 0.00 | 3.31 |
| | 155 | HIS | 8.86 | −3.52 | 0.01 | 0.00 | 12.37 |
| | 155 | HSP | 9.02 | −3.52 | 0.31 | 0.00 | 12.23 |
| * | 155 | LYS | 5.53 | −2.99 | 0.25 | 0.00 | 8.27 |
| * | 155 | ASN | 0.17 | −2.47 | −0.01 | 0.00 | 2.65 |
| ** | 155 | GLN | −1.50 | −3.63 | 0.00 | 0.00 | 2.13 |
| ** | 155 | ARG | 1.29 | −3.63 | 0.28 | 0.00 | 4.65 |
| * | 155 | SER | −0.82 | −1.77 | 0.01 | 0.00 | 0.94 |
| * | 155 | THR | 5.05 | −2.70 | 0.00 | 0.00 | 7.75 |

TABLE 10
Interferon kappa calculation results, exposed hydrophobic residues

| | # | AA | Total | vdW | Elec | Hbond | Solv |
|---|---|---|---|---|---|---|---|
| | 1 | LEU | 16.16 | −1.74 | 0.00 | 0.00 | 17.90 |
| * | 1 | ALA | 8.55 | −0.56 | 0.00 | 0.00 | 9.12 |
| * | 1 | ARG | 5.07 | −1.90 | −0.32 | 0.00 | 7.29 |
| * | 1 | ASN | 2.47 | −1.03 | 0.12 | 0.00 | 3.38 |
| ** | 1 | ASP | 0.82 | −1.11 | −0.05 | −3.98 | 5.96 |
| * | 1 | GLN | 2.37 | −1.39 | 0.03 | 0.00 | 3.73 |
| * | 1 | GLU | 3.52 | −1.14 | 0.22 | 0.00 | 4.45 |
| * | 1 | GLY | 2.79 | −0.09 | 0.00 | 0.00 | 2.88 |
| * | 1 | HIS | 10.39 | −1.90 | −0.15 | −2.54 | 14.97 |
| * | 1 | HSP | 9.14 | −1.90 | −1.03 | −2.53 | 14.61 |
| * | 1 | LYS | 7.37 | −0.82 | −0.27 | 0.00 | 8.46 |
| * | 1 | SER | 3.41 | −0.54 | 0.03 | 0.00 | 3.92 |
| * | 1 | THR | 6.26 | −1.13 | 0.03 | 0.00 | 7.37 |
| | 5 | LEU | 9.28 | −3.12 | 0.00 | 0.00 | 12.40 |
| * | 5 | ALA | 6.92 | −1.11 | 0.00 | 0.00 | 8.03 |
| * | 5 | ARG | 2.30 | −2.28 | 0.16 | 0.00 | 4.42 |
| ** | 5 | ASN | −1.00 | −1.73 | 0.02 | 0.00 | 0.71 |
| ** | 5 | ASP | −0.31 | −1.73 | −0.28 | 0.00 | 1.69 |
| * | 5 | GLN | 0.46 | −2.44 | 0.00 | 0.00 | 2.91 |
| * | 5 | GLU | 1.43 | −2.42 | −0.17 | 0.00 | 4.02 |
| * | 5 | GLY | 6.79 | −0.17 | 0.00 | 0.00 | 6.96 |
| * | 5 | HIS | 6.18 | −2.38 | −0.01 | 0.00 | 8.57 |
| * | 5 | HSP | 6.04 | −2.38 | 0.23 | 0.00 | 8.19 |
| * | 5 | LYS | 2.82 | −3.46 | 0.42 | −3.19 | 9.05 |
| * | 5 | SER | 1.03 | −1.26 | −0.01 | 0.00 | 2.29 |
| * | 5 | THR | 1.09 | −2.29 | −0.01 | 0.00 | 3.39 |
| | 8 | VAL | 5.07 | −3.35 | 0.00 | 0.00 | 8.42 |
| * | 8 | ALA | 5.02 | −1.40 | 0.00 | 0.00 | 6.43 |
| * | 8 | ARG | −0.04 | −3.23 | 0.36 | 0.00 | 2.83 |
| ** | 8 | ASN | −3.01 | −2.45 | −0.09 | −2.84 | 2.37 |
| * | 8 | ASP | −0.54 | −2.52 | −0.30 | 0.00 | 2.29 |
| ** | 8 | GLN | −2.05 | −2.96 | 0.04 | 0.00 | 0.88 |
| ** | 8 | GLU | −1.27 | −2.68 | −0.26 | 0.00 | 1.66 |
| * | 8 | GLY | 2.09 | −0.22 | 0.00 | 0.00 | 2.30 |
| * | 8 | HIS | 2.94 | −3.79 | 0.03 | 0.00 | 6.70 |
| * | 8 | HSP | 3.07 | −3.79 | 0.37 | 0.00 | 6.49 |
| * | 8 | LYS | 0.38 | −3.42 | 0.33 | 0.00 | 3.47 |
| * | 8 | SER | 0.32 | −1.69 | 0.00 | 0.00 | 2.01 |
| * | 8 | THR | 2.44 | −2.69 | 0.00 | 0.00 | 5.13 |
| | 15 | TRP | 2.66 | −6.08 | 0.00 | 0.00 | 8.74 |
| * | 15 | ALA | 2.27 | −1.39 | 0.00 | 0.00 | 3.66 |
| * | 15 | ARG | −0.49 | −3.53 | 0.41 | 0.00 | 2.63 |
| ** | 15 | ASN | −4.15 | −2.97 | 0.05 | −2.71 | 1.48 |
| ** | 15 | ASP | −3.09 | −2.99 | −0.43 | 0.00 | 0.32 |
| ** | 15 | GLN | −4.26 | −3.24 | −0.01 | 0.00 | −1.01 |
| ** | 15 | GLU | −3.94 | −3.19 | −0.36 | 0.00 | −0.37 |
| * | 15 | GLY | 1.98 | −0.30 | 0.00 | 0.00 | 2.28 |
| | 15 | HIS | 3.07 | −3.90 | 0.01 | 0.00 | 6.96 |
| | 15 | HSP | 3.13 | −3.88 | 0.42 | 0.00 | 6.59 |
| * | 15 | LYS | −0.64 | −2.80 | 0.43 | 0.00 | 1.73 |
| * | 15 | SER | −1.70 | −1.75 | −0.01 | 0.00 | 0.07 |
| | 15 | THR | 5.05 | −0.75 | 0.03 | 0.00 | 5.77 |
| | 18 | LEU | −7.96 | −6.28 | 0.00 | 0.00 | −1.69 |
| | 18 | ALA | −3.37 | −2.20 | 0.00 | 0.00 | −1.16 |
| | 18 | ARG | −3.90 | −5.75 | 0.36 | 0.00 | 1.48 |
| | 18 | ASN | −3.50 | −4.51 | 0.00 | 0.00 | 1.02 |
| | 18 | ASP | −5.98 | −4.64 | −0.35 | 0.00 | −0.99 |
| * | 18 | GLN | −7.59 | −4.63 | −0.01 | 0.00 | −2.95 |
| * | 18 | GLU | −8.87 | −5.82 | −0.43 | 0.00 | −2.61 |
| | 18 | GLY | 0.11 | −0.37 | 0.00 | 0.00 | 0.48 |
| | 18 | HIS | −0.92 | −4.87 | −0.02 | 0.00 | 3.96 |
| | 18 | HSP | 3.12 | −3.46 | 0.42 | 0.00 | 6.16 |
| * | 18 | LYS | −6.70 | −6.21 | 0.30 | 0.00 | −0.79 |
| | 18 | SER | −3.95 | −2.68 | 0.00 | 0.00 | −1.27 |
| | 18 | THR | −1.25 | −3.94 | 0.07 | 0.00 | 2.61 |
| | 28 | PHE | 18.32 | −4.71 | 0.00 | 0.00 | 23.02 |
| * | 28 | ALA | 5.85 | −1.85 | 0.00 | 0.00 | 7.69 |
| * | 28 | ARG | 3.35 | −3.31 | −0.03 | 0.00 | 6.69 |
| ** | 28 | ASN | −2.32 | −3.19 | −0.19 | −3.03 | 4.09 |
| * | 28 | ASP | 1.28 | −2.94 | 0.28 | 0.00 | 3.93 |
| * | 28 | GLN | 0.95 | −3.74 | −0.14 | −3.37 | 8.21 |
| * | 28 | GLU | 3.31 | −3.39 | 0.15 | 0.00 | 6.55 |
| * | 28 | GLY | 6.33 | −0.28 | 0.00 | 0.00 | 6.62 |
| * | 28 | HIS | 7.67 | −4.12 | 0.03 | 0.00 | 11.76 |
| * | 28 | HSP | 6.77 | −4.11 | −0.24 | 0.00 | 11.12 |
| * | 28 | LYS | 4.45 | −3.59 | −0.52 | −5.05 | 13.61 |
| * | 28 | SER | 1.76 | −2.16 | 0.01 | 0.00 | 3.91 |
| * | 28 | THR | 9.75 | 2.16 | 0.00 | 0.00 | 7.60 |
| | 30 | VAL | 10.27 | −2.35 | 0.00 | 0.00 | 12.62 |
| * | 30 | ALA | 6.08 | −0.92 | 0.00 | 0.00 | 7.00 |
| * | 30 | ARG | 2.49 | −2.42 | 0.06 | 0.00 | 4.85 |
| * | 30 | ASN | 0.13 | −1.83 | 0.00 | 0.00 | 1.97 |
| * | 30 | ASP | 1.13 | −1.82 | 0.04 | 0.00 | 2.91 |
| ** | 30 | GLN | −0.65 | −1.87 | −0.02 | 0.00 | 1.24 |
| * | 30 | GLU | 0.68 | −1.87 | 0.01 | 0.00 | 2.54 |
| * | 30 | GLY | 2.71 | −0.16 | 0.00 | 0.00 | 2.87 |
| * | 30 | HIS | 7.83 | −3.68 | −0.01 | 0.00 | 11.52 |
| * | 30 | HSP | 7.87 | −3.56 | −0.13 | 0.00 | 11.56 |
| * | 30 | LYS | 5.43 | −3.08 | 0.01 | 0.00 | 8.51 |
| * | 30 | SER | 1.64 | −1.15 | 0.00 | 0.00 | 2.78 |
| * | 30 | THR | 5.28 | −1.93 | 0.01 | 0.00 | 7.20 |
| | 33 | LEU | 8.89 | −3.10 | 0.00 | 0.00 | 12.00 |
| * | 33 | ALA | 5.67 | −0.99 | 0.00 | 0.00 | 6.67 |
| * | 33 | ARG | −0.88 | −2.82 | −0.07 | 0.00 | 2.01 |
| ** | 33 | ASN | −1.09 | −1.86 | 0.00 | 0.00 | 0.78 |
| * | 33 | ASP | 0.12 | −1.86 | 0.12 | 0.00 | 1.86 |
| ** | 33 | GLN | −3.13 | −2.90 | −0.09 | −2.65 | 2.51 |
| * | 33 | GLU | −0.44 | −2.85 | 0.16 | 0.00 | 2.24 |
| * | 33 | GLY | 2.91 | −0.15 | 0.00 | 0.00 | 3.07 |
| * | 33 | HIS | 6.16 | −2.83 | 0.01 | 0.00 | 8.98 |
| * | 33 | HSP | 5.57 | −2.83 | −0.12 | 0.00 | 8.51 |
| * | 33 | LYS | 1.75 | −2.89 | −0.09 | 0.00 | 4.73 |
| * | 33 | SER | 0.39 | −1.19 | 0.01 | 0.00 | 1.58 |
| * | 33 | THR | 1.15 | −2.27 | −0.01 | 0.00 | 3.42 |
| | 37 | ILE | 0.71 | −5.77 | 0.00 | 0.00 | 6.48 |
| | 37 | ALA | 3.26 | −1.68 | 0.00 | 0.00 | 4.94 |
| * | 37 | ARG | −1.63 | −2.56 | −0.39 | −5.88 | 7.21 |
| * | 37 | ASN | −1.24 | −3.19 | 0.03 | 0.00 | 1.92 |
| * | 37 | ASP | −3.15 | −2.98 | 0.23 | −0.10 | −0.30 |
| ** | 37 | GLN | −6.08 | −3.22 | −0.06 | −4.23 | 1.44 |
| * | 37 | GLU | −2.78 | −3.25 | 0.27 | 0.00 | 0.19 |
| | 37 | GLY | 2.71 | −0.21 | 0.00 | 0.00 | 2.92 |
| | 37 | HIS | 2.18 | −5.14 | 0.01 | 0.00 | 7.30 |
| | 37 | HSP | 2.77 | −4.28 | −0.34 | −1.12 | 8.51 |
| * | 37 | LYS | −1.72 | −4.15 | −0.21 | 0.00 | 2.64 |
| * | 37 | SER | −0.42 | −1.99 | 0.01 | 0.00 | 1.55 |
| ** | 37 | THR | −4.92 | −4.32 | 0.01 | 0.00 | −0.62 |
| | 46 | LEU | 0.03 | −4.37 | 0.00 | 0.00 | 4.40 |
| * | 46 | ALA | −2.83 | −1.86 | 0.00 | 0.00 | −0.97 |
| ** | 46 | ARG | −5.84 | −4.27 | −0.18 | −2.39 | 1.00 |
| * | 46 | ASN | −4.07 | −3.26 | 0.00 | 0.00 | −0.81 |
| ** | 46 | ASP | −6.38 | −3.22 | −0.25 | 0.00 | −2.92 |
| ** | 46 | GLN | −7.53 | −3.68 | 0.01 | 0.00 | −3.86 |
| ** | 46 | GLU | −7.16 | −3.55 | −0.12 | 0.00 | −3.48 |
| * | 46 | GLY | −0.53 | −0.26 | 0.00 | 0.00 | −0.27 |
| | 46 | HIS | 0.17 | −4.16 | −0.02 | 0.00 | 4.35 |
| * | 46 | HSP | −0.20 | −4.15 | 0.17 | 0.00 | 3.78 |
| * | 46 | LYS | −3.15 | −3.48 | 0.15 | 0.00 | 0.19 |
| ** | 46 | SER | −5.21 | −2.19 | 0.01 | 0.00 | −3.03 |
| * | 46 | THR | −0.91 | 1.44 | 0.01 | 0.00 | −2.37 |
| | 48 | TYR | −3.30 | −5.42 | 0.01 | 0.00 | 2.10 |
| | 48 | ALA | −1.88 | −1.89 | 0.00 | 0.00 | 0.01 |
| * | 48 | ARG | −5.36 | −5.53 | −0.11 | 0.00 | 0.28 |
| | 48 | ASN | −2.23 | −3.76 | −0.03 | 0.00 | 1.55 |
| ** | 48 | ASP | −9.47 | −3.96 | 0.00 | −2.99 | −2.52 |
| * | 48 | GLN | −7.50 | −4.51 | −0.11 | −2.67 | −0.22 |
| ** | 48 | GLU | −9.11 | −4.52 | −0.05 | −2.71 | −1.83 |
| | 48 | GLY | 1.29 | −0.24 | 0.00 | 0.00 | 1.52 |
| | 48 | HIS | −1.45 | −5.38 | −0.03 | 0.00 | 3.96 |
| | 48 | HSP | −2.14 | −5.37 | −0.15 | 0.00 | 3.37 |
| * | 48 | LYS | −5.37 | −4.29 | −0.11 | 0.00 | −0.96 |
| | 48 | SER | −3.16 | −2.27 | −0.01 | 0.00 | −0.88 |
| * | 48 | THR | −4.68 | −1.54 | −0.01 | 0.00 | −3.13 |
| | 52 | MET | 12.92 | −3.56 | 0.00 | 0.00 | 16.48 |
| * | 52 | ALA | 5.97 | −1.54 | 0.00 | 0.00 | 7.51 |
| * | 52 | ARG | 3.75 | −2.96 | 0.15 | 0.00 | 6.56 |
| ** | 52 | ASN | −1.71 | −1.11 | −0.27 | −5.77 | 5.43 |
| ** | 52 | ASP | −1.46 | −1.59 | −1.25 | −3.93 | 5.32 |
| * | 52 | GLN | 1.34 | −3.03 | −0.07 | 0.00 | 4.44 |
| * | 52 | GLU | 2.17 | −2.98 | −0.28 | 0.00 | 5.43 |
| * | 52 | GLY | 4.74 | −0.23 | 0.00 | 0.00 | 4.97 |
| * | 52 | HIS | 7.79 | −2.91 | −0.28 | −3.46 | 14.44 |
| * | 52 | HSP | 6.75 | −2.89 | −0.70 | −3.48 | 13.82 |
| * | 52 | LYS | 6.71 | −3.15 | 0.16 | 0.00 | 9.70 |
| * | 52 | SER | 0.84 | −1.76 | 0.04 | 0.00 | 2.56 |
| * | 52 | THR | 5.25 | −1.27 | 0.04 | 0.00 | 6.48 |
| | 65 | LEU | −2.31 | −4.75 | 0.00 | 0.00 | 2.44 |
| | 65 | ALA | −1.88 | −1.76 | 0.00 | 0.00 | −0.12 |
| * | 65 | ARG | −3.62 | −4.35 | −0.05 | 0.00 | 0.79 |
| * | 65 | ASN | −2.88 | −3.75 | 0.01 | 0.00 | 0.86 |
| * | 65 | ASP | −4.97 | −3.88 | 0.30 | 0.00 | −1.39 |
| ** | 65 | GLN | −6.92 | −4.78 | 0.03 | 0.00 | −2.18 |
| ** | 65 | GLU | −6.66 | −4.91 | 0.23 | 0.00 | −1.98 |
| | 65 | GLY | 0.31 | −0.25 | 0.00 | 0.00 | 0.56 |
| | 65 | HIS | 11.96 | 10.19 | 0.01 | 0.00 | 1.75 |
| | 65 | HSP | 13.91 | 8.82 | 0.17 | 0.00 | 4.91 |
| * | 65 | LYS | −3.12 | −4.48 | −0.18 | 0.00 | 1.54 |
| * | 65 | SER | −3.53 | −2.15 | 0.01 | 0.00 | −1.39 |
| * | 65 | THR | −4.25 | −3.45 | −0.02 | 0.00 | −0.78 |
| | 68 | PHE | −5.87 | −7.03 | 0.00 | 0.00 | 1.16 |
| | 68 | ALA | −3.75 | −2.01 | 0.00 | 0.00 | −1.74 |
| * | 68 | ARG | −6.84 | −5.85 | −0.53 | 0.00 | −0.46 |
| | 68 | ASN | −4.99 | −4.40 | −0.04 | 0.00 | −0.55 |
| * | 68 | ASP | −6.55 | −3.87 | 0.34 | 0.00 | −3.02 |
| * | 68 | GLN | −8.01 | −5.42 | −0.02 | 0.00 | −2.56 |
| ** | 68 | GLU | −9.36 | −5.40 | 0.34 | 0.00 | −4.30 |
| | 68 | GLY | −0.85 | −0.30 | 0.00 | 0.00 | −0.54 |
| * | 68 | HIS | −6.00 | −6.05 | 0.04 | 0.00 | 0.02 |
| * | 68 | HSP | −6.74 | −5.97 | −0.34 | 0.00 | −0.42 |
| ** | 68 | LYS | −9.96 | −5.89 | −0.41 | 0.00 | −3.66 |
| | 68 | SER | −3.46 | −2.41 | −0.03 | 0.00 | −1.02 |
| | 68 | THR | −2.31 | −3.42 | −0.14 | 0.00 | 1.25 |
| | 76 | PHE | 17.46 | −4.29 | 0.00 | 0.00 | 21.75 |
| * | 76 | ALA | 6.77 | −1.11 | 0.00 | 0.00 | 7.88 |
| * | 76 | ARG | 3.07 | −2.50 | −0.10 | 0.00 | 5.67 |
| ** | 76 | ASN | −1.69 | −1.48 | −0.15 | −2.30 | 2.24 |
| ** | 76 | ASP | −0.22 | −1.71 | 0.06 | 0.00 | 1.43 |
| * | 76 | GLN | 1.69 | −2.19 | −0.04 | 0.00 | 3.93 |
| * | 76 | GLU | 2.66 | −2.09 | 0.09 | 0.00 | 4.65 |
| * | 76 | GLY | 6.19 | −0.15 | 0.00 | 0.00 | 6.35 |
| * | 76 | HIS | 9.14 | −3.17 | 0.06 | 0.00 | 12.25 |
| * | 76 | HSP | 8.48 | −3.17 | −0.34 | 0.00 | 11.99 |
| * | 76 | LYS | 8.39 | −2.70 | −0.15 | 0.00 | 11.24 |
| * | 76 | SER | 0.59 | −1.28 | −0.02 | 0.00 | 1.89 |
| * | 76 | THR | 2.57 | −2.46 | −0.02 | 0.00 | 5.05 |
| | 78 | TYR | 6.54 | −5.49 | −0.04 | 0.00 | 12.07 |
| | 78 | ALA | 7.63 | −1.15 | 0.00 | 0.00 | 8.79 |
| * | 78 | ARG | 4.88 | −2.52 | −0.07 | 0.00 | 7.47 |
| * | 78 | ASN | 3.23 | −2.44 | −0.02 | 0.00 | 5.69 |
| * | 78 | ASP | 3.05 | −2.26 | 0.07 | −0.94 | 6.18 |
| ** | 78 | GLN | 1.98 | −2.21 | −0.04 | 0.00 | 4.23 |
| ** | 78 | GLU | 1.67 | −2.22 | −0.02 | 0.00 | 3.91 |
| | 78 | GLY | 6.81 | −0.14 | 0.00 | 0.00 | 6.96 |
| * | 78 | HIS | 5.82 | −6.20 | −0.02 | 0.00 | 12.03 |
| * | 78 | HSP | 3.01 | −6.07 | −0.46 | −2.67 | 12.22 |
| * | 78 | LYS | 4.97 | −3.96 | −0.48 | 0.00 | 9.41 |
| * | 78 | SER | 3.33 | −1.23 | −0.12 | −5.35 | 10.03 |
| * | 78 | THR | 2.95 | −1.98 | −0.12 | −5.18 | 10.22 |
| | 79 | TRP | 10.75 | −4.92 | 0.01 | 0.00 | 15.65 |
| * | 79 | ALA | 3.38 | −1.21 | 0.00 | 0.00 | 4.59 |
| * | 79 | ARG | 0.30 | −2.70 | −0.07 | 0.00 | 3.06 |
| ** | 79 | ASN | −1.20 | −2.37 | 0.13 | 0.00 | 1.04 |
| * | 79 | ASP | −0.65 | −2.21 | 0.26 | 0.00 | 1.31 |
| ** | 79 | GLN | −2.65 | −2.77 | −0.10 | −7.46 | 7.69 |
| * | 79 | GLU | 0.31 | −2.79 | 0.14 | 0.00 | 2.96 |
| * | 79 | GLY | 1.45 | −0.20 | 0.00 | 0.00 | 1.66 |
| * | 79 | HIS | 6.19 | −2.99 | 0.04 | 0.00 | 9.15 |
| * | 79 | HSP | 5.75 | −2.99 | −0.17 | 0.00 | 8.90 |
| * | 79 | LYS | 1.55 | −3.33 | −0.19 | 0.00 | 5.07 |
| * | 79 | SER | −0.73 | −1.40 | 0.00 | 0.00 | 0.67 |
| * | 79 | THR | 3.74 | −2.24 | −0.05 | −0.02 | 6.05 |
| | 89 | ILE | 5.42 | −4.08 | 0.00 | 0.00 | 9.50 |
| * | 89 | ALA | 3.77 | −1.15 | 0.00 | 0.00 | 4.92 |
| * | 89 | ARG | −1.59 | −4.17 | 0.11 | 0.00 | 2.48 |
| ** | 89 | ASN | −3.80 | −1.93 | 0.02 | 0.00 | −1.89 |
| ** | 89 | ASP | −3.01 | −1.82 | 0.08 | 0.00 | −1.26 |
| * | 89 | GLN | −1.06 | −2.39 | 0.10 | 0.00 | 1.23 |
| * | 89 | GLU | −0.26 | −2.18 | −0.25 | 0.00 | 2.17 |
| * | 89 | GLY | 3.72 | −0.17 | 0.00 | 0.00 | 3.89 |
| * | 89 | HIS | 4.04 | −2.39 | −0.03 | 0.00 | 6.46 |
| * | 89 | HSP | 3.42 | −2.39 | −0.14 | 0.00 | 5.96 |
| * | 89 | LYS | 3.92 | −2.39 | 0.08 | 0.00 | 6.22 |
| * | 89 | SER | −1.60 | −1.33 | 0.04 | 0.00 | −0.31 |
| * | 89 | THR | −1.68 | −2.51 | 0.04 | 0.00 | 0.79 |
| | 97 | TYR | −1.92 | −5.22 | −0.02 | 0.00 | 3.32 |
| | 97 | ALA | 0.39 | −1.49 | 0.00 | 0.00 | 1.87 |
| ** | 97 | ARG | −3.91 | −4.23 | −0.68 | −3.13 | 4.13 |
| | 97 | ASN | −1.28 | −2.95 | 0.10 | 0.00 | 1.56 |
| | 97 | ASP | −1.03 | −2.50 | 0.18 | 0.00 | 1.29 |
| * | 97 | GLN | −2.98 | −3.34 | 0.02 | 0.00 | 0.35 |
| * | 97 | GLU | −2.53 | −3.45 | 0.21 | 0.00 | 0.71 |
| | 97 | GLY | 2.13 | −0.21 | 0.00 | 0.00 | 2.33 |
| | 97 | HIS | 1.22 | −4.20 | 0.01 | 0.00 | 5.41 |
| | 97 | HSP | 0.98 | −4.21 | 0.16 | 0.00 | 5.04 |
| | 97 | LYS | −0.50 | −4.16 | −0.11 | 0.00 | 3.77 |
| | 97 | SER | 0.18 | −1.76 | −0.06 | 0.00 | 2.01 |
| ** | 97 | THR | −3.47 | −3.33 | −0.03 | 0.00 | −0.12 |
| | 112 | MET | 0.07 | −5.90 | 0.00 | 0.00 | 5.97 |
| | 112 | ALA | 3.69 | −1.52 | 0.00 | 0.00 | 5.21 |
| ** | 112 | ARG | −3.11 | −4.06 | −0.40 | −2.39 | 3.74 |
| ** | 112 | ASN | −2.04 | −2.63 | 0.01 | 0.00 | 0.58 |
| * | 112 | ASP | −1.23 | −2.33 | 0.50 | 0.00 | 0.61 |
| * | 112 | GLN | −1.40 | −2.90 | 0.09 | 0.00 | 1.42 |
| * | 112 | GLU | −1.83 | −2.95 | 0.47 | 0.00 | 0.65 |
| | 112 | GLY | 2.47 | −0.19 | 0.00 | 0.00 | 2.66 |
| | 112 | HIS | 1.58 | −4.34 | 0.02 | 0.00 | 5.90 |
| | 112 | HSP | 1.55 | −4.36 | −0.56 | 0.00 | 6.48 |
| ** | 112 | LYS | −2.09 | −3.70 | −0.37 | 0.00 | 1.99 |
| * | 112 | SER | −0.70 | −1.75 | −0.01 | 0.00 | 1.07 |
| * | 112 | THR | −0.57 | −2.95 | −0.01 | 0.00 | 2.39 |
| | 115 | MET | 20.53 | −1.89 | 0.00 | 0.00 | 22.43 |
| * | 115 | ALA | 11.10 | −0.75 | 0.00 | 0.00 | 11.85 |
| * | 115 | ARG | 8.78 | −1.98 | −0.22 | 0.00 | 10.97 |
| ** | 115 | ASN | 3.56 | −1.30 | 0.01 | 0.00 | 4.87 |
| ** | 115 | ASP | 4.09 | −0.30 | −0.30 | −2.86 | 7.55 |
| * | 115 | GLN | 6.25 | −1.40 | −0.02 | 0.00 | 7.67 |
| * | 115 | GLU | 7.28 | −1.41 | 0.17 | 0.00 | 8.52 |
| ** | 115 | GLY | 4.47 | −0.15 | 0.00 | 0.00 | 4.63 |
| * | 115 | HIS | 14.96 | −1.92 | 0.02 | 0.00 | 16.86 |
| * | 115 | HSP | 14.25 | −1.92 | −0.20 | 0.00 | 16.37 |
| * | 115 | LYS | 11.59 | −2.01 | −0.21 | 0.00 | 13.81 |
| ** | 115 | SER | 4.62 | −0.91 | 0.00 | 0.00 | 5.53 |
| * | 115 | THR | 11.38 | 0.32 | 0.00 | 0.00 | 11.06 |
| | 120 | MET | 14.72 | −3.42 | 0.00 | 0.00 | 18.15 |
| * | 120 | ALA | 10.26 | −0.70 | 0.00 | 0.00 | 10.96 |
| * | 120 | ARG | 4.52 | −2.66 | −0.24 | 0.00 | 7.42 |
| ** | 120 | ASN | 2.06 | −1.28 | −0.02 | 0.00 | 3.36 |
| ** | 120 | ASP | 3.57 | −1.28 | 0.24 | 0.00 | 4.61 |
| ** | 120 | GLN | 3.28 | −1.52 | 0.01 | 0.00 | 4.79 |
| * | 120 | GLU | 4.92 | −1.64 | 0.32 | 0.00 | 6.23 |
| * | 120 | GLY | 6.29 | −0.11 | 0.00 | 0.00 | 6.41 |
| * | 120 | HIS | 10.39 | −2.74 | −0.03 | 0.00 | 13.16 |
| * | 120 | HSP | 9.47 | −2.75 | −0.48 | 0.00 | 12.70 |
| * | 120 | LYS | 7.88 | −2.63 | −0.26 | 0.00 | 10.77 |
| * | 120 | SER | 4.15 | −0.85 | 0.02 | 0.00 | 4.98 |
| * | 120 | THR | 8.44 | −1.54 | 0.00 | 0.00 | 9.99 |
| | 127 | VAL | 7.26 | 8.43 | 0.00 | 0.00 | −1.17 |
| ** | 127 | ALA | −3.43 | −1.35 | 0.00 | 0.00 | −2.09 |
| * | 127 | ARG | 0.00 | −7.82 | −0.88 | 0.00 | 8.70 |
| ** | 127 | ASN | −4.70 | −3.66 | −0.13 | −4.04 | 3.13 |
| ** | 127 | ASP | −6.95 | −3.82 | 0.68 | −3.10 | −0.71 |
| * | 127 | GLN | −0.81 | −5.91 | −0.07 | −0.29 | 5.46 |
| ** | 127 | GLU | −3.83 | −5.90 | 0.78 | 0.00 | 1.29 |
| ** | 127 | GLY | −2.85 | −0.30 | 0.00 | 0.00 | −2.55 |
| | 127 | HIS | 16.59 | 12.31 | −0.12 | 0.00 | 4.41 |
| | 127 | HSP | 19.54 | 14.09 | −1.04 | 0.00 | 6.50 |
| * | 127 | LYS | −1.30 | −4.78 | −0.07 | 0.00 | 3.56 |
| * | 127 | SER | −0.99 | −2.21 | −0.04 | 0.00 | 1.26 |
| ** | 127 | THR | −3.15 | −4.29 | −0.04 | 0.00 | 1.17 |
| | 133 | LEU | 9.92 | −3.97 | 0.00 | 0.00 | 13.89 |
| * | 133 | ALA | 8.39 | −0.97 | 0.00 | 0.00 | 9.35 |
| * | 133 | ARG | 3.29 | −3.25 | −0.18 | 0.00 | 6.72 |
| * | 133 | ASN | 2.32 | −1.71 | −0.19 | 0.00 | 4.22 |
| * | 133 | ASP | 3.00 | −1.70 | −0.27 | 0.00 | 4.97 |
| ** | 133 | GLN | −2.05 | −2.51 | −0.14 | −5.10 | 5.69 |
| * | 133 | GLU | 2.24 | −3.06 | 0.42 | 0.00 | 4.88 |
| * | 133 | GLY | 2.12 | −0.15 | 0.00 | 0.00 | 2.27 |
| * | 133 | HIS | 9.18 | −2.46 | 0.01 | 0.00 | 11.64 |
| * | 133 | HSP | 9.02 | −2.47 | 0.30 | 0.00 | 11.19 |
| * | 133 | LYS | 3.76 | −3.26 | −0.26 | 0.00 | 7.28 |
| * | 133 | SER | 3.26 | −1.17 | −0.02 | 0.00 | 4.45 |
| * | 133 | THR | 4.07 | −2.42 | −0.04 | 0.00 | 6.53 |
| | 151 | TYR | −2.01 | −5.96 | −0.20 | −2.23 | 6.37 |
| | 151 | ALA | 2.45 | −1.62 | 0.00 | 0.00 | 4.07 |
| * | 151 | ARG | −2.32 | −3.34 | 0.09 | 0.00 | 0.94 |
| | 151 | ASN | 0.06 | −3.31 | 0.03 | 0.00 | 3.34 |
| | 151 | ASP | −1.42 | −2.87 | 0.05 | 0.00 | 1.40 |
| ** | 151 | GLN | −3.98 | −4.25 | 0.03 | 0.00 | 0.24 |
| ** | 151 | GLU | −4.41 | −4.75 | −0.09 | 0.00 | 0.43 |
| | 151 | GLY | 0.89 | −0.23 | 0.00 | 0.00 | 1.12 |
| ** | 151 | HIS | −3.72 | −5.32 | 0.02 | 0.00 | 1.58 |
| | 151 | HSP | −1.50 | −5.39 | 0.06 | 0.00 | 3.83 |
| | 151 | LYS | −1.43 | −4.88 | 0.21 | 0.00 | 3.24 |
| | 151 | SER | 0.50 | −2.12 | −0.03 | −2.79 | 5.44 |
| | 151 | THR | −0.98 | −3.30 | 0.02 | 0.00 | 2.30 |
| | 161 | VAL | −2.90 | −4.54 | 0.00 | 0.00 | 1.64 |
| | 161 | ALA | −1.30 | −1.78 | 0.00 | 0.00 | 0.48 |
| * | 161 | ARG | −5.02 | −4.50 | 0.12 | 0.00 | −0.63 |
| * | 161 | ASN | −3.65 | −3.44 | −0.21 | −1.47 | 1.46 |
| ** | 161 | ASP | −6.06 | −3.46 | −0.40 | 0.00 | −2.21 |
| * | 161 | GLN | −4.93 | −4.30 | −0.01 | 0.00 | −0.62 |
| ** | 161 | GLU | −7.22 | −4.29 | −0.28 | 0.00 | −2.66 |
| | 161 | GLY | −1.08 | −0.25 | 0.00 | 0.00 | −0.83 |
| | 161 | HIS | −1.44 | −4.70 | 0.22 | 0.00 | 3.04 |
| | 161 | HSP | −1.34 | −4.71 | 0.85 | 0.00 | 2.51 |
| * | 161 | LYS | −4.79 | −4.47 | 0.14 | 0.00 | −0.45 |
| * | 161 | SER | −2.99 | −2.12 | −0.03 | 0.00 | −0.84 |
| | 161 | THR | −0.47 | −3.87 | −0.03 | 0.00 | 3.42 |
| | 168 | TYR | 1.50 | −7.16 | −0.05 | 0.00 | 8.71 |
| | 168 | ALA | 1.77 | −1.79 | 0.00 | 0.00 | 3.56 |
| * | 168 | ARG | −0.38 | −4.14 | 0.40 | 0.00 | 3.37 |
| ** | 168 | ASN | −1.76 | −3.23 | −0.07 | −2.62 | 4.16 |
| ** | 168 | ASP | −2.08 | −3.56 | −0.38 | 0.00 | 1.85 |
| ** | 168 | GLN | −1.72 | −3.90 | −0.01 | 0.00 | 2.19 |
| ** | 168 | GLU | −1.52 | −3.79 | −0.36 | 0.00 | 2.62 |
| | 168 | GLY | 1.91 | −0.28 | 0.00 | 0.00 | 2.18 |
| | 168 | HIS | 2.66 | −5.84 | 0.00 | 0.00 | 8.51 |
| | 168 | HSP | 5.46 | −5.83 | 0.59 | 0.00 | 10.70 |
| | 168 | LYS | 2.36 | −4.49 | 0.38 | 0.00 | 6.48 |
| * | 168 | SER | −0.98 | −2.17 | −0.01 | 0.00 | 1.20 |
| * | 168 | THR | 1.15 | −3.18 | −0.01 | 0.00 | 4.34 |
| | 171 | TYR | 1.43 | −4.26 | −0.04 | 0.00 | 5.73 |
| * | 171 | ALA | −0.78 | −1.66 | 0.00 | 0.00 | 0.87 |
| * | 171 | ARG | −4.70 | −3.96 | 0.36 | 0.00 | −1.10 |
| * | 171 | ASN | −3.30 | −2.81 | −0.01 | 0.00 | −0.47 |
| ** | 171 | ASP | −5.70 | −2.80 | −0.41 | 0.00 | −2.49 |
| ** | 171 | GLN | −6.16 | −3.14 | 0.01 | 0.00 | −3.03 |
| ** | 171 | GLU | −6.10 | −4.42 | −0.32 | 0.00 | −1.35 |
| * | 171 | GLY | 0.09 | −0.22 | 0.00 | 0.00 | 0.31 |
| * | 171 | HIS | −0.40 | −5.05 | −0.06 | −0.38 | 5.09 |
| * | 171 | HSP | 1.13 | −4.02 | 0.46 | 0.00 | 4.69 |
| * | 171 | LYS | −3.45 | −5.26 | 0.43 | 0.00 | 1.38 |
| ** | 171 | SER | −4.54 | −1.92 | 0.00 | 0.00 | −2.62 |
| * | 171 | THR | −2.12 | −2.78 | 0.00 | 0.00 | 0.66 |

Next, we simultaneously designed sets of exposed hydrophobic residues that are located close to each other in space. These calculations were performed to account for coupling between interacting positions. As before, sets of residues were considered to be compatible with interferon structure if their energy was similar to or more favorable than the energy of the wild type residues at that set of positions. The most preferred sets of residues are those with the most favorable energies.

Calculations were performed on the following clusters of exposed hydrophobic residues in interferon beta: 5 and 8; 15 and 155; 22 and 148; 22, 30, 32, and 36; and 116 and 120. Results of the cluster calculations for interferon beta are given in the table below:

TABLE 11
Interferon beta calculation results, exposed hydrophobic clusters

| # | Most preferred | preferred |
|---|---|---|
| 5 | T | S, N, K, E |
| 8 | E | D, N, Q, S, R |
| 15 | D | |
| 22 | E | K, D, S, Q, R, N |
| 28 | Q | K |
| 30 | D | T, S, N, E |
| 32 | S | E |
| 36 | T | K, E |
| 116 | T | K, S, N, D, H, E |
| 120 | R | D, K, E, T, S |
| 148 | E | |
| 155 | D | E, N, S, Q |

Finally, we reconciled the results of the PDA® technology calculations and the sequence alignment data for interferon kappa. The most preferred polar substitution for each exposed hydrophobic residue was defined to be the residue with the highest normalized frequency of occurrence, among the set of polar residues with favorable energies in the PDA® technology calculations. The most preferred substitutions are: V8N, W15R, V30R, I37N, Y48Q, F76S, I89T, Y97D, M112T, M115G, V161A, Y168S, and Y171T. In the case of Y97D and V161A, the replacements have slightly less favorable energies than the wild type hydrophobic residue. However, since the energy difference is only slight and the alternate residues are frequently observed in other interferons, it is likely that these substitutions are structurally and functionally suitable.

A few of these substitutions are close in sequence to other exposed hydrophobic residues. As a result, it was possible to test the effect of altering a small number of additional residues without increasing the overall library complexity. Preferred polar residues for these additional exposed hydrophobic residues were selected for favorable PDA®) technology energies or high normalized frequency in other interferons; the most preferred substitutions are: L5Q, F28Q, M52N, Y78A, and L133Q.

### Example 6

### Identification of Suitable Replacements for Dimer Interface Residues

PDA® technology calculations were performed to identify residues that form favorable intermolecular interactions in the interferon-beta dimer. Each of the residues identified as dimer interface residues was considered. The interaction energy between each dimer interface residue in chain A and each dimer interface residue in chain B was calculated using a force field describing van der Waals interactions, electrostatics, hydrogen bonds, and solvation. The residues were all held fixed in the crystallographically observed conformations. Half-interaction energies are as shown below; the energies are symmetric and the total interaction energy is twice the value shown.

TABLE 12
Interactions across the interferon-beta dimer interface.

| | | | Glu | Glu | Gln | Leu | Gln | Gln | Gln | Arg | Leu | Met | Leu | His | Arg |
|---|---|---|---|---|---|---|---|---|---|---|---|---|---|---|---|
| | | | 42 A | 43 A | 46 A | 47 A | 48 A | 49 A | 51 A | 113 A | 116 A | 117 A | 120 A | 121 A | 124 A |
| MET | 1 | B | 0.0 | 0.0 | 0.0 | 0.0 | 0.0 | 0.0 | −1.0 | −1.4 | −0.1 | 0.0 | 0.0 | 0.0 | 0.0 |
| SER | 2 | B | 0.0 | 0.0 | 0.0 | 0.0 | 0.0 | 0.0 | 0.0 | −1.8 | −2.4 | 0.0 | 0.0 | 0.0 | 0.0 |
| TYR | 3 | B | 0.0 | 0.0 | 0.0 | 0.0 | 0.0 | 0.0 | 0.0 | 0.6 | 0.0 | 0.0 | 0.0 | 0.0 | 0.0 |
| ASN | 4 | B | 0.0 | 0.0 | 0.0 | −0.2 | 0.0 | −1.4 | 1.9 | 0.0 | 0.0 | 0.0 | 0.0 | 0.0 | 0.0 |
| LEU | 5 | B | 0.0 | −2.2 | 0.0 | −2.0 | 0.0 | 0.0 | 0.0 | 0.0 | −1.5 | −2.5 | −1.0 | −1.0 | 0.0 |
| LEU | 6 | B | 0.0 | 0.0 | 0.0 | 0.0 | 0.0 | 0.0 | 0.0 | −0.2 | −0.7 | 0.0 | 0.0 | 0.0 | 0.0 |
| PHE | 8 | B | −2.0 | −1.5 | −1.7 | −1.2 | −0.2 | 0.0 | 0.0 | 0.0 | 0.0 | 0.0 | 0.0 | 0.0 | 0.0 |
| LEU | 9 | B | −0.7 | −1.8 | 0.0 | −0.1 | 0.0 | 0.0 | 0.0 | 0.0 | −1.0 | −0.3 | −2.4 | −3.3 | 0.0 |
| SER | 12 | B | 0.2 | 0.0 | 1.0 | 0.0 | 0.0 | 0.0 | 0.0 | 0.0 | 0.0 | 0.0 | 0.0 | 0.0 | 0.0 |
| GLN | 16 | B | 0.9 | 0.0 | 0.0 | 0.0 | 0.0 | 0.0 | 0.0 | 0.0 | 0.0 | 0.0 | 0.0 | 0.0 | 0.0 |
| HIS | 93 | B | 0.0 | 0.0 | 0.0 | 0.0 | 0.0 | 0.0 | 0.0 | 0.0 | 0.0 | 0.0 | −0.8 | −2.1 | 0.9 |
| ASN | 96 | B | 0.0 | 0.0 | 0.0 | 0.0 | 0.0 | 0.0 | 0.0 | 0.0 | 0.0 | 0.0 | −0.4 | 0.0 | 1.0 |
| HIS | 97 | B | 0.0 | 0.0 | 0.0 | 0.0 | 0.0 | 0.0 | 0.0 | 0.0 | −0.8 | 0.0 | −2.4 | −2.0 | 1.9 |
| THR | 100 | B | 0.0 | 0.0 | 0.0 | 0.0 | 0.0 | 0.0 | 0.0 | 0.3 | −1.7 | 0.0 | −0.7 | 0.0 | 0.0 |
| VAL | 101 | B | 0.0 | 0.0 | 0.0 | 0.0 | 0.0 | 0.0 | 0.0 | 0.6 | −1.6 | 0.0 | 0.0 | 0.0 | 0.0 |
| GLU | 104 | B | 0.0 | 0.0 | 0.0 | 0.0 | 0.0 | 0.0 | 0.0 | −2.6 | −0.5 | 0.0 | 0.0 | 0.0 | 0.0 |

Residues that participate in at least one intermolecular interaction that is at least 1 kcal/mol in magnitude may play a role in dimer formation; those residues that form several favorable interactions are especially likely to be critical for dimerization.,

Next, SPA calculations were used to identify suitable replacements for the dimer interface residues. Two sets of calculations were performed for each interface residue. First, the energy of the most favorable rotamer for each possible residue was determined in the context of the monomer structure (chain A or chain B, PDB code 1AU1). Next, the energy of the most favorable rotamer for each possible residue was determined in the context of the dimer structure (chains A and B, PDB code 1AU1). These energies were analyzed to identify residues that are compatible with the monomer structure but not the dimer structure. Residues were deemed compatible with the monomer structure if their energy score in the monomer structure was better than 2, and residues were deemed incompatible with the dimer structure if their energy score in the dimer structure was worse than 2.

TABLE 13
SPA energies in the context of the monomer structure.
The residue number and chain identifier are shown in the left, along with the
residue observed in wild type interferon beta. Energy scores were truncated at 50.0.

| | | | A | C | D | E | F | G | H | I | K | L |
|---|---|---|---|---|---|---|---|---|---|---|---|---|
| 42 | A | E | 0.5 | 2.0 | 0.3 | 0.9 | 3.0 | 3.8 | 3.1 | 1.5 | 1.5 | 1.4 |
| 43 | A | E | 1.4 | 1.9 | 2.9 | 1.3 | 1.1 | 6.6 | 3.0 | 1.8 | 0.9 | 0.0 |
| 46 | A | Q | 0.9 | 1.9 | 1.7 | 0.6 | 1.8 | 4.1 | 2.2 | 11.2 | 0.4 | 1.1 |
| 47 | A | L | 3.6 | 4.0 | 4.2 | 1.7 | 20.0 | 6.8 | 5.7 | 20.0 | 1.4 | 3.9 |
| 48 | A | Q | 1.7 | 2.8 | 1.1 | 1.6 | 4.3 | 4.6 | 3.3 | 2.1 | 2.1 | 2.9 |
| 49 | A | Q | 1.0 | 2.1 | 0.5 | 0.8 | 3.4 | 3.3 | 2.8 | 3.7 | 1.9 | 2.3 |
| 51 | A | Q | 1.0 | 2.8 | 3.5 | 1.3 | 3.2 | 4.9 | 2.5 | 4.0 | 1.0 | 1.9 |
| 113 | A | R | 0.9 | 1.8 | 1.5 | 0.5 | 1.5 | 3.4 | 1.7 | 2.6 | 1.1 | 1.5 |
| 116 | A | L | 0.3 | 2.0 | 1.4 | 0.0 | 2.7 | 4.1 | 3.4 | 1.7 | 1.2 | 1.0 |
| 117 | A | M | 2.2 | 4.0 | 5.1 | 8.0 | 19.7 | 7.7 | 12.9 | 1.1 | 4.7 | 7.3 |
| 120 | A | L | 1.9 | 2.9 | 1.5 | 2.2 | 2.1 | 4.5 | 3.4 | 9.4 | 1.4 | 1.8 |
| 121 | A | H | 1.5 | 3.1 | 1.9 | 1.6 | 1.5 | 5.6 | 2.9 | 20.0 | 0.1 | 1.6 |
| 124 | A | R | 0.3 | 1.6 | 1.3 | 0.0 | 4.0 | 4.2 | 1.7 | 0.7 | 1.0 | 0.9 |
| 1 | B | M | 0.5 | 2.0 | 0.4 | 0.5 | 3.9 | 2.8 | 2.9 | 3.4 | 1.5 | 2.4 |
| 2 | B | S | 4.1 | 4.6 | 4.3 | 3.9 | 5.5 | 0.0 | 4.0 | 3.9 | 2.4 | 4.7 |
| 3 | B | Y | 5.7 | 5.8 | 7.3 | 5.8 | 2.1 | 9.2 | 5.5 | 11.9 | 4.2 | 4.2 |
| 4 | B | L | 1.9 | 2.4 | 0.5 | 0.6 | 4.5 | 5.4 | 5.2 | 1.5 | 1.9 | 2.8 |
| 5 | B | L | 0.5 | 1.8 | 0.3 | 0.0 | 2.4 | 4.4 | 2.7 | 0.7 | 1.0 | 0.6 |
| 6 | B | L | 5.4 | 7.0 | 6.4 | 5.5 | 20.0 | 10.1 | 12.3 | 20.0 | 5.5 | 0.0 |
| 7 | B | G | 50.0 | 50.0 | 50.0 | 50.0 | 50.0 | 0.0 | 50.0 | 50.0 | 50.0 | 50.0 |
| 8 | B | F | 0.8 | 1.9 | 1.2 | 0.0 | 2.4 | 4.5 | 3.0 | 5.9 | 1.5 | 0.9 |
| 9 | B | L | 2.3 | 3.5 | 4.0 | 2.5 | 7.0 | 7.6 | 3.7 | 1.4 | 0.3 | 0.0 |
| 12 | B | S | 0.3 | 1.2 | 0.3 | 0.3 | 1.8 | 4.4 | 3.4 | 0.5 | 0.8 | 0.3 |
| 16 | B | Q | 0.0 | 1.5 | 0.0 | 0.3 | 4.7 | 4.5 | 1.8 | 0.3 | 0.4 | 1.1 |
| 93 | B | H | 0.1 | 1.7 | 1.7 | 0.5 | 5.3 | 4.3 | 1.6 | 0.7 | 0.4 | 0.1 |
| 96 | B | N | 1.3 | 2.0 | 1.6 | 0.0 | 3.0 | 5.2 | 2.0 | 0.6 | 0.6 | 0.0 |
| 97 | B | H | 1.6 | 3.1 | 3.4 | 2.3 | 6.5 | 7.1 | 2.7 | 0.0 | 1.5 | 3.8 |
| 100 | B | T | 0.9 | 2.2 | 2.4 | 1.1 | 2.8 | 5.0 | 2.8 | 0.7 | 0.8 | 0.0 |
| 101 | B | V | 2.4 | 3.6 | 4.5 | 9.2 | 20.0 | 8.3 | 8.9 | 1.4 | 3.9 | 13.0 |
| 104 | B | E | 1.7 | 3.6 | 4.5 | 1.3 | 4.6 | 5.4 | 3.6 | 3.2 | 0.4 | 0.8 |

| M | N | P | Q | R | S | T | V | W | Y |
|---|---|---|---|---|---|---|---|---|---|
| 2.3 | 0.1 | 0.0 | 0.4 | 1.3 | 0.1 | 0.5 | 2.1 | 5.4 | 2.7 |
| 1.8 | 2.5 | 2.0 | 1.2 | 0.7 | 2.2 | 1.1 | 0.6 | 3.7 | 1.5 |
| 2.7 | 0.0 | 50.0 | 0.0 | 0.4 | 0.6 | 2.1 | 8.5 | 5.7 | 1.4 |
| 2.4 | 2.6 | 50.0 | 0.0 | 2.5 | 3.7 | 7.5 | 20.0 | 50.0 | 50.0 |
| 2.9 | 0.0 | 3.9 | 0.9 | 2.3 | 1.2 | 1.2 | 2.9 | 7.0 | 3.7 |
| 3.4 | 0.0 | 4.9 | 0.5 | 1.4 | 0.2 | 1.6 | 2.9 | 5.8 | 3.3 |
| 3.3 | 1.0 | 0.0 | 0.9 | 1.3 | 0.5 | 3.2 | 3.2 | 5.6 | 3.2 |
| 2.0 | 0.0 | 50.0 | 0.3 | 0.3 | 0.2 | 1.8 | 2.2 | 5.0 | 1.4 |
| 2.8 | 0.5 | 50.0 | 0.1 | 1.5 | 0.2 | 0.7 | 1.8 | 5.4 | 3.0 |
| 3.3 | 3.7 | 5.0 | 6.9 | 1.8 | 2.9 | 1.7 | 0.0 | 20.0 | 13.8 |
| 2.8 | 0.0 | 17.7 | 2.6 | 2.6 | 2.1 | 3.9 | 8.2 | 5.9 | 1.7 |
| 2.6 | 0.0 | 20.0 | 0.9 | 0.8 | 1.9 | 1.1 | 10.2 | 4.2 | 1.8 |
| 2.1 | 1.0 | 50.0 | 0.5 | 1.3 | 0.4 | 0.9 | 0.5 | 6.5 | 4.0 |
| 3.4 | 0.1 | 3.6 | 0.2 | 0.9 | 0.0 | 1.7 | 2.6 | 6.5 | 3.7 |
| 4.4 | 2.5 | 50.0 | 3.3 | 3.4 | 3.3 | 2.1 | 6.3 | 7.8 | 6.3 |
| 3.8 | 5.4 | 50.0 | 6.0 | 8.2 | 6.0 | 14.7 | 12.9 | 0.0 | 2.5 |
| 3.7 | 0.0 | 5.8 | 1.1 | 2.3 | 1.2 | 1.5 | 1.4 | 6.5 | 4.8 |
| 1.6 | 0.4 | 5.5 | 0.2 | 0.6 | 0.4 | 0.6 | 0.3 | 4.1 | 2.3 |
| 4.5 | 6.0 | 50.0 | 6.3 | 16.6 | 7.4 | 10.8 | 50.0 | 20.0 | 20.0 |
| 50.0 | 50.0 | 50.0 | 50.0 | 50.0 | 50.0 | 50.0 | 50.0 | 50.0 | 50.0 |
| 3.2 | 0.6 | 50.0 | 0.2 | 1.3 | 0.8 | 2.6 | 9.5 | 4.3 | 2.9 |
| 2.1 | 3.1 | 50.0 | 2.2 | 3.2 | 1.1 | 2.7 | 2.5 | 8.8 | 7.0 |
| 1.4 | 0.0 | 50.0 | 0.1 | 1.0 | 0.6 | 0.7 | 0.9 | 2.9 | 2.3 |
| 0.9 | 0.7 | 50.0 | 0.6 | 1.9 | 0.1 | 1.3 | 0.4 | 7.5 | 4.5 |
| 1.9 | 0.9 | 50.0 | 0.0 | 1.0 | 0.4 | 0.8 | 1.3 | 8.1 | 4.7 |
| 2.0 | 1.7 | 50.0 | 0.3 | 1.3 | 1.2 | 1.7 | 1.6 | 5.9 | 3.4 |
| 2.8 | 0.1 | 50.0 | 2.6 | 2.6 | 1.8 | 2.0 | 0.0 | 8.1 | 10.4 |
| 2.4 | 1.5 | 50.0 | 0.6 | 0.8 | 1.3 | 1.6 | 1.8 | 6.5 | 3.1 |
| 7.9 | 4.0 | 50.0 | 9.9 | 6.4 | 3.5 | 2.0 | 0.0 | 20.0 | 20.0 |
| 2.1 | 2.7 | 50.0 | 0.0 | 1.4 | 0.0 | 1.0 | 4.1 | 7.8 | 4.9 |

TABLE 14
SPA energies in the context of the dimer structure.
The residue number and chain identifier are shown in the left, along with the
residue observed in wild type interferon beta. Energy scores were truncated at 50.0.

| | | | A | C | D | E | F | G | H | I | K | L |
|---|---|---|---|---|---|---|---|---|---|---|---|---|
| 42 | A | E | 0.9 | 2.6 | 1.0 | 1.3 | 2.8 | 4.9 | 3.4 | 0.6 | 1.2 | 0.9 |
| 43 | A | E | 0.5 | 1.7 | 6.2 | 2.5 | 20.0 | 7.0 | 8.0 | 0.9 | 3.0 | 7.7 |
| 46 | A | Q | 0.7 | 1.9 | 1.9 | 0.4 | 1.0 | 4.5 | 2.0 | 20.0 | 0.0 | 0.5 |
| 47 | A | L | 4.0 | 4.3 | 4.1 | 1.7 | 14.0 | 8.3 | 3.8 | 20.0 | 1.4 | 1.9 |
| 48 | A | Q | 1.7 | 2.6 | 0.9 | 1.6 | 3.8 | 4.6 | 3.2 | 1.9 | 2.2 | 2.7 |
| 49 | A | Q | 1.4 | 2.9 | 0.8 | 2.3 | 2.5 | 4.8 | 2.9 | 3.0 | 2.5 | 3.9 |
| 51 | A | Q | 1.2 | 2.7 | 3.6 | 1.9 | 2.1 | 5.5 | 3.2 | 3.9 | 1.2 | 1.5 |
| 113 | A | R | 1.7 | 3.4 | 4.1 | 2.2 | 0.0 | 5.1 | 1.0 | 2.0 | 0.0 | 0.3 |
| 116 | A | L | 1.9 | 3.3 | 4.4 | 2.3 | 0.0 | 6.9 | 2.7 | 1.3 | 1.7 | 3.0 |
| 117 | A | M | 2.3 | 4.3 | 5.1 | 7.2 | 20.0 | 8.1 | 15.5 | 3.0 | 6.6 | 7.1 |
| 120 | A | L | 1.6 | 2.7 | 1.9 | 2.3 | 0.7 | 4.7 | 2.6 | 8.0 | 0.9 | 0.6 |
| 121 | A | H | 2.5 | 3.9 | 3.0 | 2.3 | 3.0 | 6.7 | 3.4 | 20.0 | 0.3 | 1.9 |
| 124 | A | R | 0.4 | 1.6 | 1.4 | 0.0 | 3.8 | 4.3 | 1.9 | 0.9 | 1.2 | 0.9 |
| 1 | B | M | 0.4 | 1.9 | 0.7 | 1.2 | 2.1 | 3.3 | 3.1 | 3.1 | 0.5 | 1.7 |
| 2 | B | S | 2.9 | 3.0 | 5.9 | 9.3 | 12.8 | 0.0 | 5.7 | 6.0 | 5.8 | 20.0 |
| 3 | B | Y | 5.9 | 6.0 | 6.4 | 5.5 | 2.3 | 9.4 | 5.6 | 12.2 | 5.2 | 4.4 |
| 4 | B | N | 2.4 | 2.9 | 0.2 | 1.6 | 8.6 | 6.9 | 6.9 | 2.0 | 2.1 | 2.0 |
| 5 | B | L | 4.0 | 5.7 | 5.2 | 6.7 | 3.4 | 9.8 | 3.8 | 0.0 | 5.3 | 6.9 |
| 6 | B | L | 5.4 | 7.0 | 6.5 | 4.9 | 20.0 | 10.1 | 14.0 | 20.0 | 5.9 | 0.0 |
| 7 | B | G | 50.0 | 50.0 | 50.0 | 50.0 | 50.0 | 0.0 | 50.0 | 50.0 | 50.0 | 50.0 |
| 8 | B | F | 4.9 | 6.0 | 7.3 | 4.4 | 0.0 | 9.8 | 4.7 | 17.5 | 4.1 | 5.2 |
| 9 | B | L | 2.9 | 4.7 | 5.9 | 4.2 | 2.8 | 8.5 | 2.6 | 1.9 | 0.0 | 0.1 |
| 12 | B | S | 0.1 | 1.5 | 0.7 | 7.3 | 9.1 | 4.9 | 16.5 | 2.0 | 5.9 | 6.0 |
| 16 | B | Q | 0.1 | 1.6 | 0.3 | 0.7 | 4.7 | 4.6 | 2.0 | 0.3 | 0.0 | 1.1 |
| 93 | B | H | 0.0 | 1.7 | 1.1 | 0.0 | 5.4 | 4.3 | 1.6 | 0.6 | 0.7 | 0.0 |
| 96 | B | N | 1.4 | 2.0 | 1.6 | 0.1 | 3.1 | 5.3 | 1.8 | 0.8 | 1.0 | 0.0 |
| 97 | B | H | 1.9 | 3.4 | 3.4 | 2.7 | 5.3 | 7.6 | 2.8 | 0.0 | 1.5 | 3.4 |
| 100 | B | T | 1.1 | 2.6 | 2.3 | 1.3 | 1.8 | 5.5 | 2.6 | 0.7 | 1.3 | 0.0 |
| 101 | B | V | 2.0 | 2.6 | 3.1 | 9.0 | 20.0 | 7.9 | 15.0 | 18.3 | 6.5 | 20.0 |
| 104 | B | E | 2.0 | 3.4 | 4.3 | 2.6 | 2.8 | 6.4 | 5.6 | 3.2 | 0.0 | 7.9 |

| M | N | P | Q | R | S | T | V | W | Y |
|---|---|---|---|---|---|---|---|---|---|
| 2.6 | 0.8 | 0.0 | 0.2 | 2.2 | 1.0 | 1.0 | 1.8 | 5.5 | 2.8 |
| 2.6 | 5.7 | 0.2 | 2.7 | 11.8 | 2.1 | 0.9 | 0.0 | 20.0 | 20.0 |
| 2.4 | 0.3 | 50.0 | 0.1 | 0.3 | 0.5 | 4.8 | 20.0 | 5.0 | 0.8 |
| 1.3 | 2.6 | 50.0 | 0.0 | 3.7 | 4.8 | 8.0 | 20.0 | 50.0 | 50.0 |
| 2.8 | 0.0 | 4.0 | 0.9 | 2.0 | 1.0 | 1.0 | 2.9 | 6.0 | 3.4 |
| 3.6 | 0.0 | 4.3 | 2.4 | 1.9 | 1.6 | 2.3 | 2.3 | 4.3 | 2.6 |
| 2.8 | 2.1 | 0.0 | 1.6 | 1.7 | 0.7 | 3.6 | 3.4 | 2.0 | 1.7 |
| 2.6 | 0.8 | 50.0 | 1.7 | 0.3 | 1.7 | 2.3 | 2.0 | 2.7 | 0.3 |
| 2.0 | 3.7 | 50.0 | 2.9 | 5.1 | 1.3 | 0.9 | 1.6 | 20.0 | 1.8 |
| 3.3 | 4.0 | 4.9 | 6.9 | 4.8 | 3.1 | 1.5 | 0.0 | 20.0 | 20.0 |
| 1.7 | 0.0 | 19.0 | 2.9 | 3.4 | 2.0 | 2.1 | 7.0 | 3.4 | 0.3 |
| 2.4 | 0.0 | 20.0 | 2.3 | 2.1 | 2.5 | 1.1 | 10.6 | 12.3 | 8.9 |
| 2.1 | 1.2 | 50.0 | 0.7 | 1.4 | 0.3 | 0.9 | 0.5 | 6.3 | 4.3 |
| 3.0 | 0.1 | 2.9 | 1.0 | 0.5 | 0.0 | 1.4 | 1.7 | 5.8 | 4.2 |
| 6.4 | 4.2 | 50.0 | 17.7 | 11.0 | 2.3 | 1.5 | 4.2 | 20.0 | 8.9 |
| 4.0 | 7.2 | 50.0 | 6.3 | 9.3 | 6.5 | 15.3 | 12.6 | 0.0 | 2.2 |
| 3.0 | 0.0 | 6.1 | 1.9 | 3.7 | 2.2 | 2.4 | 2.7 | 50.0 | 9.3 |
| 4.0 | 4.6 | 8.4 | 8.4 | 10.1 | 5.0 | 3.5 | 1.1 | 20.0 | 4.4 |
| 4.4 | 6.1 | 50.0 | 6.3 | 17.9 | 7.3 | 11.0 | 50.0 | 20.0 | 20.0 |
| 50.0 | 50.0 | 50.0 | 50.0 | 50.0 | 50.0 | 50.0 | 50.0 | 50.0 | 50.0 |
| 4.9 | 5.9 | 50.0 | 3.7 | 8.0 | 6.1 | 5.7 | 13.8 | 10.2 | 5.6 |
| 2.1 | 4.9 | 50.0 | 3.6 | 4.3 | 1.6 | 3.8 | 3.2 | 20.0 | 3.1 |
| 4.8 | 0.4 | 50.0 | 7.4 | 7.6 | 0.9 | 1.2 | 0.0 | 9.8 | 8.4 |
| 1.2 | 0.9 | 50.0 | 0.6 | 0.5 | 0.1 | 1.2 | 0.3 | 6.0 | 4.7 |
| 1.5 | 1.0 | 50.0 | 0.1 | 1.6 | 0.7 | 0.9 | 1.1 | 8.9 | 4.6 |
| 2.1 | 2.0 | 50.0 | 0.5 | 2.1 | 1.2 | 1.8 | 1.6 | 5.7 | 3.5 |
| 2.1 | 0.8 | 50.0 | 2.9 | 3.8 | 2.5 | 2.3 | 0.5 | 20.0 | 20.0 |
| 2.5 | 2.1 | 50.0 | 1.1 | 1.6 | 1.9 | 1.9 | 1.9 | 6.1 | 2.5 |
| 12.3 | 3.3 | 50.0 | 10.5 | 10.3 | 3.4 | 1.5 | 0.0 | 20.0 | 20.0 |
| 2.5 | 4.3 | 50.0 | 1.6 | 3.0 | 0.1 | 0.6 | 3.6 | 3.6 | 4.2 |

TABLE 15
Suitable replacements for dimer interface positions,
as determined by the above SPA calculations.

| | | | A | C | D | E | F | G | H | I | K | L | M | N | P | Q | R | S | T | V | W | Y |
|---|---|---|---|---|---|---|---|---|---|---|---|---|---|---|---|---|---|---|---|---|---|---|
| 42 | A | E | | | | | | | | | | | | | | | | | | | | |
| 43 | A | E | | | | | F | | | | K | L | | | | | R | | | | | Y |
| 46 | A | Q | | | | | | | | | | | | | | | | | | | | |
| 47 | A | L | | | | | | | | | | | | | | | | | | | | |
| 48 | A | Q | | | | | | | | | | | | | | | | | | | | |
| 49 | A | Q | | | | | | | | | | | | | | | | | | | | |
| 51 | A | Q | | | | | | | | | | | | | | | | | | | | |
| 113 | A | R | | | D | | | | | | | | | | | | | | | | | |
| 116 | A | L | | | D | E | | | | | | L | | N | | Q | R | | | | | |
| 117 | A | M | | | | | | | | | | | | | | | R | | | | | |
| 120 | A | L | | | | | | | | | | | | | | | | | | | | |
| 121 | A | H | | | | | | | | | | | | | | | | | | | | Y |
| 124 | A | R | | | | | | | | | | | | | | | | | | | | |
| 1 | B | M | | | | | | | | | | | | | | | | | | | | |
| 2 | B | S | | | | | | | | | | | | | | | | | | | | |
| 3 | B | Y | | | | | | | | | | | | | | | | | | | | |
| 4 | B | L | | | | | | | | | | | | | | | | | | | | |
| 5 | B | L | A | C | D | E | | | | | K | L | M | N | | Q | R | S | T | | | |
| 6 | B | L | | | | | | | | | | | | | | | | | | | | |
| 7 | B | G | | | | | | | | | | | | | | | | | | | | |
| 8 | B | F | A | C | D | E | | | | | K | L | | N | | Q | R | S | | | | |
| 9 | B | L | | | | | | | | | | | | | | | | | | | | |
| 12 | B | S | | | | E | F | | | | K | L | M | | | Q | R | | | | | |
| 16 | B | Q | | | | | | | | | | | | | | | | | | | | |
| 93 | B | H | | | | | | | | | | | | | | | | | | | | |
| 96 | B | N | | | | | | | | | | | | | | | | | | | | |
| 97 | B | H | | | | | | | | | | | | | | | | | | | | |
| 100 | B | T | | | | | | | | | | | | | | | | | | | | |
| 101 | B | V | | | | | | | | I | | | | | | | | | | | | |
| 104 | B | E | | | | | | | | | | L | | | | | | | | | | |

As can be observed in the tables above, positions 5, 8, 12, 43, and 116 are all involved in stabilizing the dimer structure of interferon-beta, and a number of modifications at these positions are predicted to significantly prevent dimerization.

Further analysis was performed to determine which of the above modifications is most likely to significantly prevent dimerization. Hydrophobic interactions and electrostatic interactions (including salt bridges and hydrogen bonds) can stabilize protein-protein interfaces. These interactions may be effectively disrupted by hydrophobic to polar and charge reversal mutations.

Hydrophobic residues that are significantly less solvent exposed in the dimer structure versus the monomer structure were defined to be those residues that are classified as surface in the monomer and core or boundary in the dimer, and residues that are classified as boundary in the monomer and core in the dimer, as shown below:

TABLE 16
Hydrophobic residues that are more buried in the dimer
than in the monomer.

| Residue | Monomer | Dimer |
|---|---|---|
| Leu 5 | Boundary | Core |
| Phe 8 | Surface | Core |
| Leu 9 | Boundary | Core |
| Leu 47 | Boundary | Core |
| Leu 116 | Surface | Boundary |

Debye-Huckel scaled Coulomb's law calculations were performed on the 1AU1 dimer and monomers, using an ionic strength of 0.15 M, to determine the electrostatic potential at each position in the context of the monomer versus the dimer. The following positions were found to have a change in potential of at least 0.20 kcal/mol:

TABLE 17
Positions that experience a significant difference
in electrostatic potential in the dimer versus monomer structure.

| | | | Dimer | Monomer | Difference |
|---|---|---|---|---|---|
| SER | 2 | B | 0.36 | −0.30 | 0.66 |
| LEU | 5 | B | −0.10 | 0.11 | −0.21 |
| PHE | 8 | B | 0.14 | 0.42 | −0.28 |
| LEU | 9 | B | −0.11 | 0.16 | −0.27 |
| SER | 12 | B | −0.42 | 0.29 | −0.71 |
| LEU | 47 | A | 0.25 | 0.04 | 0.21 |
| GLN | 49 | A | 0.32 | 0.08 | 0.24 |
| HIS | 93 | B | 0.29 | 0.04 | 0.25 |
| ASN | 96 | B | 0.24 | 0.04 | 0.20 |
| THR | 100 | B | −0.22 | −0.45 | 0.23 |
| VAL | 101 | B | 0.15 | −0.39 | 0.54 |
| GLU | 104 | B | 0.58 | −0.02 | 0.60 |
| ARG | 113 | A | −1.37 | −0.36 | −1.01 |

Modifications of the electrostatic properties of the residues at these positions can be selected to favor the monomer structure and disfavor the dimer structure. For example, Glu 104 and Arg 113 form a salt bridge in the dimer structure, which can be observed in the crystal structure. In the table above, Glu 104 is in a region of positive potential in the dimer and neutral potential in the monomer, while Arg 113 is in a region of negative potential in the dimer structure and slightly negative potential in the monomer structure. Modifications that could disrupt this interaction include, but are not limited to, E104R, E104K, E104H, E104Q, E104A, R113D, R113E, R113Q, and R113A.

### Example 7

### Identification of Suitable Replacements for Free Cysteine Residues

PDA® technology calculations were also performed to identify suitable replacements for free cysteine residues. These calculations were performed using the methods described above for the hydrophobic to polar point mutations, except that both polar and nonpolar replacements were considered. Alternate residues with favorable energies are marked with a star (*) below.

TABLE 18
Free cysteine calculation results

| | AA | Total | VDW | Elec | HBond | Solv |
|---|---|---|---|---|---|---|
| | IFNa | | | | | |
| | TYR-C | −13.47 | −10.45 | −0.11 | −2.32 | −0.59 |
| | ILE | 15.37 | 13.90 | 0.00 | 0.00 | 1.47 |
| * | LEU | −5.58 | −5.38 | 0.00 | 0.00 | −0.20 |
| * | MET | −6.17 | −5.42 | 0.00 | 0.00 | −0.75 |
| | PHE | 887.53 | 893.12 | 0.00 | 0.00 | −5.59 |
| | TRP | 0.98 | −6.86 | −0.01 | 0.00 | 7.86 |
| | TYR | 803.08 | 804.33 | −0.02 | 0.00 | −1.23 |
| | VAL | 27.93 | 29.08 | 0.00 | 0.00 | −1.15 |
| | ALA | −2.53 | −1.89 | 0.00 | 0.00 | −0.63 |
| * | ASP | −4.45 | −4.05 | 0.33 | 0.00 | −0.73 |
| * | GLU | −7.53 | −4.66 | 0.39 | 0.00 | −3.26 |
| * | HIS | −5.94 | −6.12 | −0.12 | 0.00 | 0.30 |
| * | HSP | −4.19 | −5.94 | −0.76 | 0.00 | 2.51 |
| * | LYS | −8.48 | −5.48 | −0.38 | 0.00 | −2.63 |
| | ASN | −3.00 | −4.12 | −0.03 | 0.00 | 1.15 |
| * | GLN | −8.21 | −4.70 | −0.01 | 0.00 | −3.50 |
| * | ARG | −4.73 | −5.42 | −0.24 | 0.00 | 0.93 |
| | SER | −4.04 | −2.17 | −0.02 | 0.00 | −1.85 |
| * | THR | −5.10 | −3.08 | −0.02 | 0.00 | −2.01 |
| | IFNb | | | | | |
| | CYS-C | −13.97 | −7.06 | 0.00 | 0.00 | −6.91 |
| | ILE | 324.91 | 334.90 | 0.00 | 0.00 | −9.99 |
| | LEU | 840.30 | 846.29 | 0.00 | 0.00 | −5.99 |
| | MET | 2082.91 | 2089.08 | 0.00 | 0.00 | −6.17 |
| | PHE | 5529.90 | 5539.67 | 0.00 | 0.00 | −9.77 |
| | TYR | 6341.29 | 6346.98 | −0.26 | 0.00 | −5.43 |
| | VAL | 82.62 | 89.33 | 0.00 | 0.00 | −6.70 |
| * | ALA | −8.69 | −3.42 | 0.00 | 0.00 | −5.27 |
| * | ASP | −10.20 | −7.37 | 0.12 | 0.00 | −2.96 |
| | GLU | 357.99 | 358.18 | 0.42 | 0.00 | −0.62 |
| | HIS | 501.55 | 504.61 | −0.05 | 0.00 | −3.01 |
| | HSP | 506.45 | 506.93 | 0.35 | 0.00 | −0.83 |
| | LYS | 2087.79 | 2085.18 | −0.04 | 0.00 | 2.64 |
| * | ASN | −5.08 | −6.54 | 0.11 | 0.00 | 1.36 |
| | GLN | 483.14 | 479.27 | 0.10 | 0.00 | 3.77 |
| | ARG | 15093.59 | 15085.56 | 0.04 | 0.00 | 7.99 |
| * | SER | −5.96 | −4.41 | −0.08 | 0.00 | −1.47 |
| * | THR | −9.17 | −5.20 | 0.06 | 0.00 | −4.03 |
| | IFNk | | | | | |
| | LEU-C | 5507.86 | 5514.27 | −0.41 | 0.00 | −6.01 |
| | ILE | 44.93 | 50.89 | 0.00 | 0.00 | −5.96 |
| | LEU | −13.20 | −7.12 | 0.00 | 0.00 | −6.08 |
| * | MET | −3.21 | 3.30 | 0.00 | 0.00 | −6.51 |
| | PHE | 36.05 | 43.81 | 0.00 | 0.00 | −7.76 |
| | TRP | 292.31 | 298.19 | −0.01 | 0.00 | −5.87 |
| | TYR | 196.77 | 200.15 | −0.01 | 0.00 | −3.37 |
| | VAL | 37.53 | 42.27 | 0.00 | 0.00 | −4.74 |
| * | ALA | −7.83 | −2.63 | 0.00 | 0.00 | −5.20 |
| | ASP | −4.81 | −5.70 | −0.12 | 0.00 | 1.01 |
| * | GLU | −9.02 | −8.02 | −0.17 | 0.00 | −0.83 |
| * | HIS | −10.31 | −9.00 | −0.11 | 0.00 | −1.21 |
| * | HSP | −7.47 | −8.25 | −0.23 | 0.00 | 1.00 |
| | LYS | 2.43 | 0.20 | 0.02 | 0.00 | 2.22 |
| | ASN | −0.48 | −5.83 | 0.00 | 0.00 | 5.35 |
| * | GLN | −4.21 | −7.92 | −0.03 | 0.00 | 3.74 |
| | ARG | 52.67 | 44.39 | 0.01 | 0.00 | 8.27 |
| * | SER | −4.86 | −3.32 | 0.00 | 0.00 | −1.54 |
| * | THR | −3.56 | −3.63 | −0.10 | 0.00 | 0.18 |

### Example 8

### Generation of Interferon Beta Variants

Construction of the Interferon Beta Gene as a Template for Mutagenesis

The DNA sequence, GenBank accession number NM_{—}002176, encompassing the full-length human interferon beta cDNA gene containing the native signal sequence was modified to remove the signal sequence and facilitate high level expression in bacterial cells. Primers were designed to synthesize the region between positions 65-561 by recursive PCR. The primer sequences also biased the codon usage towards highly expressed E. coli bacterial genes. In addition, the codon for cysteine 17 (amino acid numbering with the signal sequence removed) was changed to serine. An internal SacI DNA restriction enzyme site was designed for ease of later mutagenesis as well as NdeI and XhoI restriction sites flanking the ends of the gene for cassette cloning into various expression vectors. The bacterial expression vectors pET28a and pET24a (Novagen) were used to sub-clone the interferon beta gene containing C17S between the NdeI and XhoI multiple cloning restriction sites. Cloning into pET24a expression in E. coli produces a C17S interferon beta variant while cloning into pET28a introduces the additional amino acid sequence MGSSHHHHHHSSGLVPRGSH to the N-terminus of C17S. This amino acid sequence includes a 6-His purification tag and a thrombin cleavage site for later removal of the added amino acid sequences.

Construction of Interferon Beta Variants Containing Exposed Hydrophobic to Polar Mutations

Sixteen solvent exposed hydrophobic residues were identified in the interferon beta structure. Polar amino acid residues to substitute at these positions were designed by computational analysis as described above. The list of substitutions are listed in the table below:

TABLE 19
List of substitutions used in library of interferon beta variants

| position | wt | LIB |
|---|---|---|
| 5 | L | Q |
| 8 | F | E |
| 15 | F | D |
| 22 | W | E |
| 28 | L | Q |
| 30 | Y | N |
| 32 | L | E |
| 36 | M | K |
| 47 | L | K |
| 92 | Y | D |
| 111 | F | N |
| 116 | L | E |
| 120 | L | R |
| 130 | L | T |
| 148 | V | E |
| 155 | Y | S |

Mutagenesis experiments were done to construct variants containing these amino acid substitutions in in interferon beta-C17S gene background (referred to as “wild type” throughout the following examples).

For a library containing combinations of the wild-type or substitution listed in the table above, a template directed ligation-PCR method was used as described in Strizhov et. al. PNAS 93:15012-15017 (1996). Varients constructed contain single or multiple combinations of the substitutions.

For a 64-member library containing all possible combinations of wild-type or above-listed substitution at positions 5, 8, 47, 111, 116, and/or 120, multiple rounds of site-directed mutagenesis reactions were done using the Quikchange kit (commercially available from Stratagene) following the manufacturer's protocol. Positive clones were identified by sequencing.

Production of Interferon Beta Variants in E. coli

Sequence verified clones in pET28a were transformed into BL21 (DE3) star cells (commercially available from Invitrogen) and cultures were grown in auto-inducing media, a rich medium for growth with little or no induction during log phase and auto-induction of expression as the culture approaches saturation. Media components include 25 mM (NH₄)₂SO₄, 50 mM KH₂PO₄, 50 mM Na₂HPO₄, 1 mM MgSO₄, 0.5% glycerol, 0.05% glucose, 0.2% alpha-lactose, 0.1% tryptone, and 0.05% yeast extract. The cultures were grown for 7 hours to an OD between 4 and 5 and cells harvested by centrifugation. Cells were lysed by sonication, inclusion pellets denatured in 8M guanidine HCl and bound to a column containing Ni-NTA resin. A dilution series of guanidine HCl with decreasing pH was used to purify and refold the protein.

An alternative method for purification of clones with and without the N-terminal 6-His tag was followed as disclosed in U.S. Pat. No. 4,462,940, Lin et al, Meth. Enzymol. 119:183-192.

### Example 9

### Soluble Expression of Interferon Beta Variants

Each of the 64 members of the library described above were tested for soluble expression. Western blot analysis utilizing an anti-His antibody was done for the soluble fractions of cell lysates. A band running at the expected size of approximately 20 kilodaltons was present for at least 33 of the variants but was not detectable for the C17S variant, suggesting that many of the designed variants exhibit improved soluble expression.

### Example 10

### Activity Analysis of Constructed Variants

A standard ISRE (interferon-stimulated response element) reporter assay was used to determine the activity of interferon beta variants. In this assay, 293T cells which constitutively express the type I interferon receptor were transiently transfected with an ISRE-luciferase vector (pISRE-luc, commercially available from Clontech). Twelve hours after transfection, the cells were treated with a dilution series of concentrations for an interferon beta variant. Variants which bind the interferon receptor and trigger the JAK/STAT signal transduction cascade activate transcription of the luciferase gene operably linked to the ISRE. Luciferase activity was detected using the Steady-Glo® Luciferase Assay System (commercially available from Promega) with the TopCount NXT™ microplate reader used to measure luminescence.

Initial activity determination utilizing the ISRE reporter assay was done for the 64 member library described in example 8. Cultures were grown, cells harvested and lysed. The inclusion pellet was resuspended in a 0.025% SDS solution and tested in the ISRE activity assay. Activity was demonstrated for the 37 variants listed in the table below. However, since the amount of protein tested in this assay was not quantitated first, it is possible that additional variants are active but were present in insufficient quantity to be detected in the assay.

TABLE 20
Amino acid sequences at exposed hydrophobic
positions for active interferon beta variants

Amino acid position

| Variant | 5 | 8 | 47 | 111 | 116 | 120 |
|---|---|---|---|---|---|---|
| IFB1_2 | Q | F | L | F | L | L |
| IFB1_3 | Q | F | K | F | L | L |
| IFB1_4 | L | E | L | F | L | L |
| IFB1_5 | L | E | K | F | L | L |
| IFB1_6 | L | F | K | F | L | L |
| IFB1_7 | Q | E | L | F | L | L |
| IFB1_8 | Q | E | K | F | L | L |
| IFB1_9 | L | F | L | N | L | L |
| IFB1_10 | Q | F | L | N | L | L |
| IFB1_11 | Q | F | K | N | L | L |
| IFB1_15 | Q | E | L | N | L | L |
| IFB1_16 | Q | E | K | N | L | L |
| IFB1_23 | Q | E | L | F | E | L |
| IFB1_26 | Q | F | L | F | L | R |
| IFB1_27 | Q | F | K | F | L | R |
| IFB1_28 | L | E | L | F | L | R |
| IFB1_29 | L | E | K | F | L | R |
| IFB1_31 | Q | E | L | F | L | R |
| IFB1_32 | Q | E | K | F | L | R |
| IFB1_33 | L | F | L | N | E | L |
| IFB1_34 | Q | F | L | N | E | L |
| IFB1_35 | Q | F | K | N | E | L |
| IFB1_36 | L | E | L | N | E | L |
| IFB1_37 | L | E | K | N | E | L |
| IFB1_39 | Q | E | L | N | E | L |
| IFB1_40 | Q | E | K | N | E | L |
| IFB1_41 | L | F | L | N | L | R |
| IFB1_42 | Q | F | L | N | L | R |
| IFB1_44 | L | E | L | N | L | R |
| IFB1_47 | Q | E | L | N | L | R |
| IFB1_48 | Q | E | K | N | L | R |
| IFB1_50 | Q | F | L | F | E | R |
| IFB1_51 | Q | F | K | F | E | R |
| IFB1_52 | L | E | L | F | E | R |
| IFB1_55 | Q | E | L | F | E | R |
| IFB1_56 | Q | E | K | F | E | R |
| IFB1_63 | Q | E | L | N | E | R |
| IFB1_64 | Q | E | K | N | E | R |

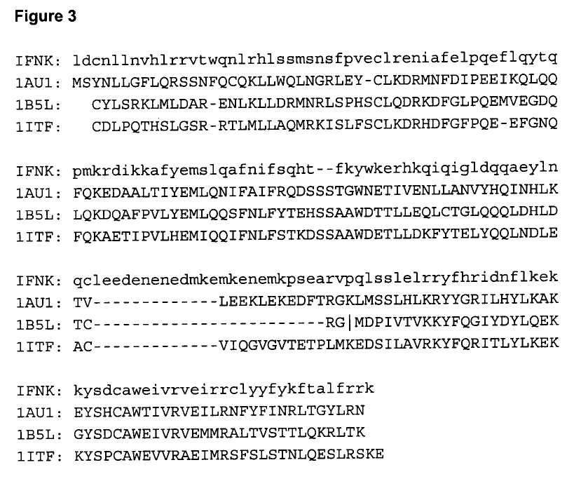

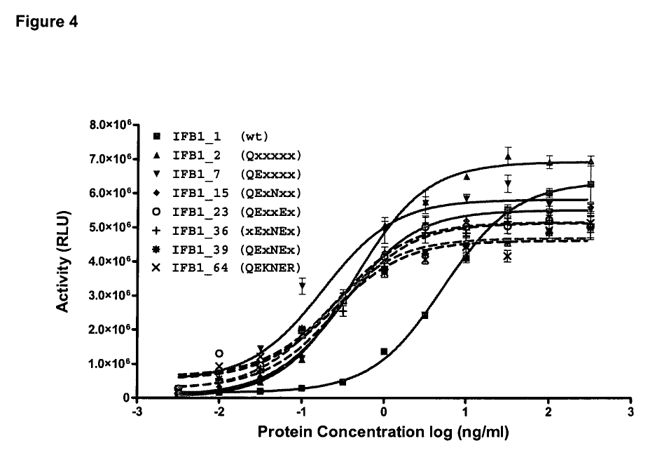

Those variants exhibiting increased activity relative to the wild type (interferon beta C17S) were tested for more quantitative activity measurements. Selected variants were purified and refolded as described in example 8 above. Each variant was then assayed using a ten point half-log dilution series in the ISRE reporter assay. GraphPad Prism®, version 4 (GraphPad Software, Inc.) was used to plot the data and calculate EC50 values. The dose response curves for the retested variants are shown in FIG. 4. All of the variants exhibited improved activity, with EC50 values ranging from 12-30 fold better activity than C17S interferon beta, as shown in the table below.

TABLE 21
Specific activity data for interferon-beta variants.
The sequence for residues 5, 8, 47, 111, 116, and 120
is given for each variant, along with the total number
of mutations, the EC50, and the ratio of the wild type to
variant EC50. Variant IFN1_1 is the
interferon beta wild type with C17S.

| | | | | | | | | EC50 | |
|---|---|---|---|---|---|---|---|---|---|
| | | | | | | | | (log | |
| | | | | | | | | ng/ | EC50 wt/ |
| Variant | 5 | 8 | 47 | 111 | 116 | 120 | # mut | ml) | EC50 var |
| IFN1_1 | L | F | L | F | L | L | 0 | 5.306 | 1.0 |
| IFB1_2 | Q | F | L | F | L | L | 1 | 0.428 | 12.4 |
| IFB1_7 | Q | E | L | F | L | L | 2 | 0.179 | 29.6 |
| IFB1_15 | Q | E | L | N | L | L | 3 | 0.319 | 16.6 |
| IFB1_23 | Q | E | L | F | E | L | 3 | 0.277 | 19.2 |
| IFB1_36 | L | E | L | N | E | L | 3 | 0.294 | 18.0 |
| IFB1_39 | Q | E | L | N | E | L | 4 | 0.193 | 27.5 |
| IFB1_64 | Q | E | K | N | E | R | 6 | 0.240 | 22.1 |

Activity Comparison With Claimed Solubility Mutant from U.S. Pat. No. 6,572,853.

Several variants with enhanced solubility were claimed in U.S. Pat. No. 6,572,853. Activity comparison of one of these claimed variants with the C17S wild type and the most active variant tested above was done. Purification of all the variants and activity evaluation was done under the same conditions with the results shown in the table below. The claimed solubility variant (IFB_GM2) exhibited 67 fold less activity than the wild type C17S interferon beta. In comparison, variant IFB1_{—}7 still exhibited better than 25 fold better activity than the wild type.

TABLE 22
Specific activity data for interferon-beta variants.
The sequence for residues 5, 8, 47, 50, 106, 111, 116,
and 120 is given for each variant, along with the total
number of mutations, the EC50, and the ratio of the wild
type to variant EC50. All variants are in the C17S background.

| Variant | 5 | 8 | 47 | 50 | 106 | 111 | 116 | 120 | # mut | EC50 (ng/ml) | EC50 wt/EC50 var |
|---|---|---|---|---|---|---|---|---|---|---|---|
| IFN1_1 | L | F | L | F | L | F | L | L | 0 | 1.90 | 1.00 |
| IFB1_7 | Q | E | L | F | L | F | L | L | 2 | 0.074 | 25.7 |
| IFB_GM2 | L | F | S | S | S | S | S | S | 6 | 130 | 0.015 |

### Example 11

### Mutagenesis, Expression, and Soluble Expression Screening of Interferon Kappa

Construction of Interferon Kappa Variants

Interferon kappa variants (total library size=1024) with the mutations listed in the table below (single and all possible multiple combinations) were constructed essentially as described above for the Interferon beta variants.

TABLE 23
List of substitutions used in library of interferon-kappa variants.
Each position or set of positions could have either the wild type
hydrophobic residue(s) or the alternate polar residue(s) listed in
the “LIB” column.

| position(s) | wt | LIB |
|---|---|---|
| 5-8 | L-V | Q-N |
| 15 | W | R |
| 28-30 | F-V | Q-R |
| 37 | I | N |
| 48-52 | Y-M | Q-N |
| 76-78 | F-Y | S-A |
| 89 | I | T |
| 97 | Y | D |
| 161 | V | A |
| 166-168-171 | C-Y-Y | A-S-T |

Expression and Screening for Soluble Expression Via Dot-Blot Using Anti-His Antibodies for Detection.

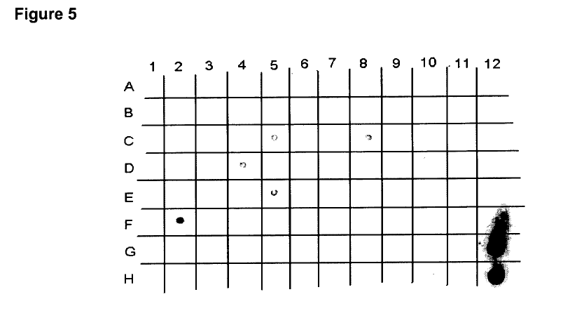

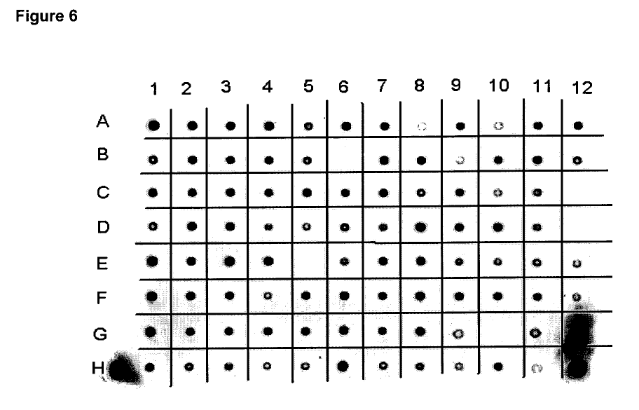

The soluble fraction of E. coli lysates expressing individual interferon-kappa variants were dot-blotted on nitrocellulose membranes, and the presence of soluble His-tagged protein was detected using anti-His antibodies conjugated to HRP. FIG. 5 shows the results of a dot-blot analysis. The positive clones expressing soluble interferon-kappa were regrown, and expressed protein was retested to confirm soluble expression. FIG. 6 shows a retest plate.

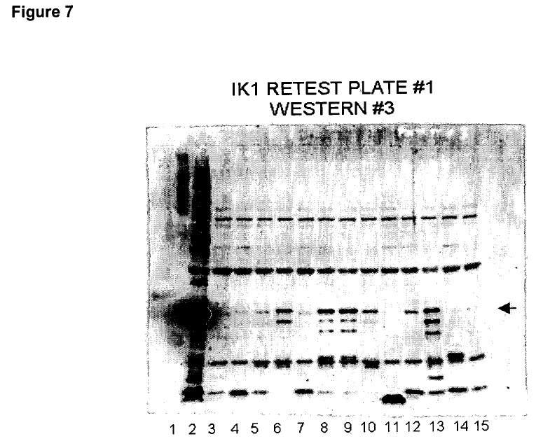

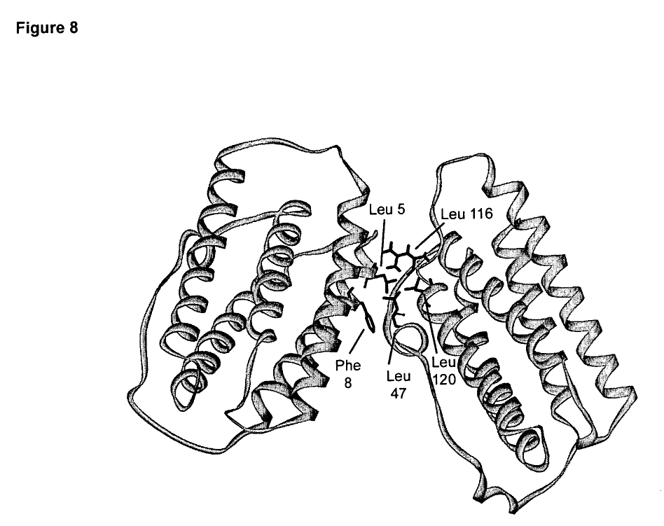

The soluble extract from interferon-kappa variants testing positive during the secondary screen were then analyzed by SDS-PAGE/Western blotting to confirm the presence of the correctly sized protein band. FIG. 7 is an example of these SDS-PAGE/Western blot experiments, identifying several interferon-kappa variants expressing the correctly sized protein with solubility characteristics better than WT interferon-kappa. The arrow indicates the expected position of interferon-kappa protein. Lanes 2 and 3 are total soluble fraction from WT interferon-kappa expressing cells, respectively. Lanes 4-15 are soluble fractions from the lysates of different variants.

TABLE 24
Sequence analysis of selected interferon kappa
variants with improved soluble expression.

| | WT Seq | | | | | | | | | |
|---|---|---|---|---|---|---|---|---|---|---|
| | L-V | W | F-V | I | Y-M | F-Y | I | Y | V | C-Y-Y |
| | Mutation | | | | | | | | | |
| | Q-N | R | Q-R | N | Q-N | S-A | T | D | A | A-S-T |
| | Mutant | | | | | | | | | |
| | 5, 8 | 15 | 28, 30 | 37 | 48, 52 | 76, 78 | 89 | 97 | 161 | 166, 168, 171 |
| IK_4-G7 | L-N | R | F-V | I | Q-N | S-A | T | Y | V | C-Y-Y |
| IK_12-E4 | L-N | R | F-V | I | Q-N | S-A | T | Y | V | C-Y-Y |
| IK_2-C11 | L-N | R | Q-R | N | Y-M | S-A | T | D | A | A-S-T |
| IK_10-D8 | L-N | W | F-V | I | Q-N | F-Y | T | D | V | A-S-T |
| IK_10-H7 | L-N | W | F-V | I | Q-N | S-A | T | D | A | A-S-T |
| IK_20-B12 | L-N | W | Q-R | I | Q-N | S-A | T | Y | V | A-S-T |
| IK_3-A11 | L-N | W | Q-R | I | Y-M | S-A | T | D | A | A-S-T |
| IK_3-H7 | L-N | W | Q-R | I | Y-M | S-A | T | D | A | A-S-T |
| IK_12-F11 | L-N | W | Q-R | N | Q-N | S-A | T | Y | V | A-S-T |
| IK_3-D10 | L-V | R | F-V | I | Q-N | S-A | T | D | V | A-S-T |
| IK_3-C10 | L-V | R | F-V | I | Q-N | S-A | T | D | V | C-Y-Y |
| IK_3-H11 | L-V | R | F-V | I | Q-N | S-A | T | D | V | C-Y-Y |
| IK_21-E1 | L-V | R | F-V | I | Y-M | S-A | I | D | V | A-S-T |
| IK_4-H11 | L-V | R | F-V | I | Y-M | S-A | T | D | A | C-Y-Y |
| IK_3-A2 | L-V | R | F-V | I | Y-M | S-A | T | D | V | A-S-T |
| IK_10-D2 | L-V | R | F-V | N | Y-M | S-A | T | D | V | C-Y-Y |
| IK_12-H4 | L-V | W | F-V | I | Q-N | S-A | I | Y | V | C-Y-Y |
| IK_27-A6 | L-V | W | F-V | I | Q-N | S-A | T | D | A | C-Y-Y |
| IK_2-B4 | L-V | W | F-V | I | Q-N | S-A | T | D | V | C-Y-Y |
| IK_3-F11 | L-V | W | F-V | I | Q-N | S-A | T | D | V | C-Y-Y |
| IK_14-A9 | L-V | W | F-V | I | Y-M | F-Y | T | Y | V | C-Y-Y |
| IK_19-A5 | L-V | W | F-V | I | Y-M | S-A | I | D | A | C-Y-Y |
| IK_3-G10 | L-V | W | F-V | I | Y-M | S-A | I | D | V | C-Y-Y |
| IK_4-A2 | L-V | W | F-V | I | Y-M | S-A | I | D | V | C-Y-Y |
| IK_4-A10 | L-V | W | F-V | I | Y-M | S-A | I | D | V | C-Y-Y |
| IK_16-G2 | L-V | W | F-V | I | Y-M | S-A | T | D | A | C-Y-Y |
| IK_22-A4 | L-V | W | F-V | I | Y-M | S-A | T | D | V | A-S-T |
| IK_1-C8 | L-V | W | F-V | N | Q-N | S-A | I | D | V | C-Y-Y |
| IK_23-C10 | L-V | W | F-V | N | Q-N | S-A | I | D | V | C-Y-Y |
| IK_12-H11 | L-V | W | F-V | N | Q-N | S-A | T | Y | V | C-Y-Y |
| IK_9-H4 | L-V | W | Q-R | N | Y-M | S-A | I | D | V | A-S-T |

Variants with improved soluble expression were tested for activity using the ISRE assay, essentially as in the initial activity assay described above. A number of variants that retain interferon activity were identified, including those listed below.

TABLE 25
Sequence analysis of some of the Interferon-kappa variant, which still retain
activity, as tested in an ISRE assay as described above for interferon beta.

| | WT seq | | | | | | | | | |
|---|---|---|---|---|---|---|---|---|---|---|
| | L-V | W | F-V | I | Y-M | F-Y | I | Y | V | C-Y-Y |
| | Mutations | | | | | | | | | |
| | Q-N | R | Q-R | N | Q-N | S-A | T | D | A | A-S-T |
| | Variant | | | | | | | | | |
| | 5, 8 | 15 | 28, 30 | 37 | 48, 52 | 76, 78 | 89 | 97 | 161 | 166, 168, 171 |
| IK1_4_G7 | L-N | R | F-V | I | Q-N | S-A | T | Y | V | C-Y-Y |
| IK1_46_E2 | L-V | R | F-V | N | Q-N | S-A | T | D | A | A-S-T |
| IK1_47_C4 | L-V | R | F-V | I | Y-M | S-A | I | Y | V | C-Y-Y |
| IK1_23_C10 | L-V | W | F-V | N | Q-N | S-A | I | D | V | C-Y-Y |
| IK1_40_A10 | L-V | R | F-V | N | Y-M | S-A | I | Y | V | C-Y-Y |

We claim:

1. A variant type I interferon protein exhibiting improved solubility relative to a wild type interferon protein selected from the group consisting of SEQ ID NOs: 1-18.

2. A variant type 1 interferon protein according to claim 1 wherein said variant type 1 interferon protein maintains at least one biological activity selected from the group consisting of immunomodulatory, antiviral, and antineoplastic activities.

3. A variant type I interferon according to claim 1 wherein said variant interferon differs from a naturally occurring interferon of SEQ ID NOs: 1-18 by at least one substitution of a solvent-exposed hydrophobic residue.

4. A variant type I interferon according to claim 1 wherein said variant interferon is incapable of dimer formation.

5. A variant type I interferon according to claim 1 wherein said variant interferon has reduced immunogenicity as compared to a wild type interferon of SEQ ID NOS: 1-18.

6. A variant interferon according to claim 1 wherein said variant interferon is derived from an interferon-alpha selected from the group consisting of SEQ ID NOs: 1-14.

7. A variant interferon according to claim 1 wherein said variant interferon is derived from the interferon-beta of SEQ ID NO: 15.

8. A variant interferon according to claim 1 wherein said variant interferon is derived from the interferon-kappa of SEQ ID NO: 16.

9. A variant interferon according to claim 6 comprising modifications selected from at least one of the following positions: 16, 27, 30, 89, 100, 110, 111, 117, 128, and 161, wherein said modifications are substitution mutations selected from the group consisting of alanine, arginine, aspartic acid, asparagine, glutamic acid, glutamine, glycine, histidine, serine, threonine, and lysine.

10. A variant interferon according to claim 7 comprising modifications selected from at least one of the following positions: 5, 8, 15, 22, 28, 30, 32, 36, 47, 92, 111, 116, 120, 130, 148, and 155, wherein said modifications to residues 5, 8, 15, 47, 111, 116, and 120 are substitution mutations selected from the group consisting of alanine, arginine, aspartic acid, asparagine, glutamic acid, glutamine, glycine, histidine, and lysine, and said modifications to residues 22, 28, 30, 32, 36, 92, 130, 148, and 155 are selected from the group including alanine, arginine, aspartic acid, asparagine, glutamic acid, glutamine, glycine, histidine, serine, threonine and lysine.

11. A variant type I interferon according to claim 10 comprising at least one modification selected from the group consisting of: L5Q, F8E, W22E, L28Q, Y30H, L32A, L47K, Y92Q, F111N, L116D, L116E, L120D, L120R, L130R, V148A, and Y155S.

12. A variant type I interferon according to claim 11 comprising at least one modification selected from the group consisting of: L5Q, F8E, L47K, F111N, L116E, and L120R.

13. A variant interferon according to claim 1 comprising SEQ ID NO: 19.

14. A variant type I interferon according to claim 1 comprising SEQ ID NO: 20.

15. A variant type I interferon according to claim 1 comprising SEQ ID NO: 21.

16. A variant type I interferon according to claim 1 comprising SEQ ID NO: 22.

17. A variant type I interferon according to claim 1 comprising SEQ ID NO: 23.

18. A variant type I interferon according to claim 1 comprising SEQ ID NO: 24.

19. A variant type I interferon according to claim 1 comprising SEQ ID NO: 25.

20. A variant type I interferon according to claim 8 comprising at least one modification at the following positions: 1, 5, 8, 15, 18, 28, 30, 33, 37, 46, 48, 52, 65, 68, 76, 79, 89, 97, 112, 115, 120, 127, 133, 151, 161, 168, and 171, wherein said modifications are substitution mutations selected from the group consisting of alanine, arginine, aspartic acid, asparagine, glutamic acid, glutamine, glycine, histidine, serine, threonine, and lysine.

21. A variant type I interferon according to claim 20 comprising at least one modification selected from the group consisting of: L5Q, V8N, W15R, F28Q, F28S, V30R, I37N, Y48Q, M52N, M52Q, F76S, Y78A, I89T, Y97D, M112T, M115G, L133Q, V161A, C166A, Y168S, and Y171T.

22. A variant type I interferon according to claim 21 comprising SEQ ID NO:26.

23. A variant type I interferon according to claim 20 comprising SEQ ID NO:27.

24. A variant type I interferon according to claim 20 comprising SEQ ID NO:28.

25. A variant type I interferon according to claim 20 comprising SEQ ID NO:29.

26. A variant type I interferon according to claim 20 comprising SEQ ID NO: 30.

27. A variant type I interferon according to claim 3 wherein said interferon is interferon-beta and comprises at least one modification selected from a modification at a position selected from the group consisting of: 1, 2, 3, 4, 5, 6, 8, 9, 12, 16, 42, 43, 46, 47, 48, 49, 51, 93, 96, 97, 100, 101, 104, 113, 116, 117, 120, 121, and 124.

28. A variant type I interferon according to claim 27 comprising at least one modification at a position selected from the group consisting of: L5A, L5D, L5E, L5K, L5N, L5Q, L5R, L5S, L5T, F8A, F8D, F8E, F8K, F8N, F8Q, F8R, F8S, S12E, S12K, S12Q, S12R, E43K, E43R, E104R, E104K, E104H, E104Q, E104A, R113D, R113E, R113Q, R113A, L116D, L116E, L116N, L116Q, L116R, and M117R.

29. A recombinant nucleic acid encoding a variant interferon selected from claim 1.

30. An expression vector comprising the nucleic acid of claim 29.

31. A host cell comprising the recombinant nucleic acid of claim 29.

32. A host cell comprising the expression vector of claim 30.

33. A method of producing a variant interferon comprising culturing the host cell of claim 32 under conditions suitable for expression of said nucleic acid.

34. A method according to claim 33 further comprising recovering said variant interferon.

35. A pharmaceutical composition comprising a variant type I interferon of claim 1 and a pharmaceutical carrier.

36. A method of treating an interferon-responsive condition in a patient needing said treatment comprising administering the pharmaceutical composition of claim 35.

37. A method of inhibiting interferon dimer formation comprising contacting a variant interferon of claim 1 with a wild type interferon of SEQ ID NOs: 1-18.


SEQUENCE LISTING (90 sequences)

SEQ ID NO: 1 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 2 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 3 - protein, 166 aa, Homo sapiens [sequence omitted]

SEQ ID NO: 4 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 5 - protein, 189 aa, Homo sapiens
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 6 - protein, 189 aa, Homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 7 - protein, 189 aa, Homo sapiens
MARSFSLLMAVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLTEKKYSPCAWEVVRAEIMRSFSFSTNLKKGLRRKD

SEQ ID NO: 8 - protein, 189 aa, Homo sapiens
MALTFYLMVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLEVLCDQEVGVIESPLMYEDSILAVRKYFQRITLYLTEKKYSSCAWEVVRAEIMRSFSLSINLQKRLKSKE

SEQ ID NO: 9 - protein, 189 aa, Homo sapiens
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 10 - protein, 189 aa, Homo sapiens
MASPFALLMALVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 11 - protein, 189 aa, Homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSFSFSTNLQKRLRRKD

SEQ ID NO: 12 - protein, 189 aa, Homo sapiens
MALSFSLLMAVLVLSYKSICSLGCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 13 - protein, 189 aa, Homo sapiens
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 14 - protein, 189 aa, Homo sapiens
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDMEACVIQEVGVEETPLMNVDSILAVKKYFQRITLYLTEKKYSPCAWEVVRAEIMRSFSLSKIFQERLRRKE

SEQ ID NO: 15 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 16 - protein, 207 aa, Homo sapiens
MSTKPDMIQKCLWLEILMGIFIAGTLSLDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 17 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 18 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 19 - protein, 166 aa, Artificial
  synthetic
MSYNQLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 20 - protein, 166 aa, Artificial
  synthetic
MSYNQLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 21 - protein, 166 aa, Artificial
  synthetic
MSYNQLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 22 - protein, 166 aa, Artificial
  synthetic
MSYNQLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSLHLKRYYGRILHYLKAKEYSHCAWTIVRVEILRNFYFINRLTGYLRN

SEQ ID NO: 23 - protein, 166 aa, Artificial
  synthetic
MSYNLLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 24 - protein, 166 aa, Artificial
  synthetic
MSYNQLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 25 - protein, 166 aa, Artificial
  synthetic
MSYNQLGELQRSSNFQSQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSRHLKRYYGRILHYLKAKEYSHCAWTIVRVEILRNFYFINRLTGYLRN

SEQ ID NO: 26 - protein, 180 aa, Artificial
  synthetic
LDCNLLNNHLRRVTRQNLRHLSSMSNSFPVECLRENIAFELPQEFLQQTQPNKRDIKKAFYEMSLQAFNIFSQHTSKAWKERHLKQIQTGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 27 - protein, 180 aa, Artificial
  synthetic
LDCNLLNVHLRRVTRQNLRHLSSMSNSFPVECLRENNAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 28 - protein, 180 aa, Artificial
  synthetic
LDCNLLNVHLRRVTRQNLRHLSSMSNSFPVECLRENIAFELPQEFLQYTQPMKRDIKKAFYEMSLQAFNIFSQHTSKAWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 29 - protein, 180 aa, Artificial
  synthetic
LDCNLLNVHLRRVTWQNLRHLSSMSNSFPVECLRENNAFELPQEFLQQTQPNKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 30 - protein, 180 aa, Artificial
  synthetic
LDCNLLNVHLRRVTRQNLRHLSSMSNSFPVECLRENNAFELPQEFLQYTQPMKRDIKKAFYEMSLQAFNIFSQHTSKAWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 31 - DNA, 757 bp, Homo sapiens
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagattttaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtccctctgggactggacaattgcttcaagcattcttcaaccagcagatgctgtttaagtgactgatggctaatgtactgcaaatgaaaggacactagaagattttgaaatttttattaaattatgagttatttttatttatttaaattttattttggaaaataaattatttttggtgc

SEQ ID NO: 32 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 33 - protein, 166 aa, Homo sapiens
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 34 - protein, 186 aa, Homo sapiens
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNMGSSHHHHHHSSGLVPRGSH

SEQ ID NO: 35 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 36 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 37 - protein, 166 aa, Artificial
  synthetic
MSYNLLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 38 - protein, 166 aa, Artificial
  synthetic
MSYNLLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 39 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 40 - protein, 166 aa, Artificial
  synthetic
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 41 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 42 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 43 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 44 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 45 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 46 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 47 - protein, 166 aa, Artificial
  synthetic
MSYNLLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 48 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 49 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 50 - protein, 166 aa, Artificial
  synthetic
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 51 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 52 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 53 - protein, 166 aa, Artificial
  synthetic
MSYNLLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 54 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 55 - protein, 166 aa, Artificial
  synthetic
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 56 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 57 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 58 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 59 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 60 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 61 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 62 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 63 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 64 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 65 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSRHLKRYYGRILHYLKAKEYSHCAWTIVRVEILRNFYFINRLTGYLRN

SEQ ID NO: 66 - protein, 166 aa, Artificial
  synthetic
MSYNLLGFLQRSSNFQCQKLLWQLNGRLEYCLKDRMNFDIPEEIKQSQQSQKEDAALTIYEMLQNIFAIFRQDSSSTGWNETIVENLLANVYHQINHLKTVLEEKSEKEDSTRGKSMSSSHLKRYYGRILHYLKAKEYSHCAWTIVRVEILRNFYFINRLTGYLRN

SEQ ID NO: 67 - protein, 180 aa, Homo sapiens
LDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 68 - protein, 180 aa, Artificial
  synthetic
LDCNLLNNHLRRVTRQNLRHLSSMSNSQPRECLRENNAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 69 - protein, 180 aa, Artificial
  synthetic
LDCNLLNNHLRRVTWQNLRHLSSMSNSFPVECLRENIAFELPQEFLQQTQPNKRDIKKAFYEMSLQAFNIFSQHTFKYWKERHLKQIQTGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 70 - protein, 180 aa, Artificial
  synthetic
LDCNLLNNHLRRVTWQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 71 - protein, 180 aa, Artificial
  synthetic
LDCNLLNNHLRRVTWQNLRHLSSMSNSQPRECLRENIAFELPQEFLQQTQPNKRDIKKAFYEMSLQAFNIFSQHTSKAWKERHLKQIQTGLDQQAEYLNQCLEEDENENEDMKEMKENEMKPSEARVPQLSSLELRRYFHRIDNFLKEKKYSDCAWEIVRVEIRRALSYFTKFTALFRRK

SEQ ID NO: 72 - protein, 180 aa, Artificial
  synthetic
LDCNLLNNHLRRVTWQNLRHLSSMSNSQPRECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 73 - protein, 180 aa, Artificial
  synthetic
LDCNLLNNHLRRVTWQNLRHLSSMSNSQPRECLRENNAFELPQEFLQQTQPNKRDIKKAFYEMSLQAFNIFSQHTSKAWKERHLKQIQTGLDQQAEYLNQCLEEDENENEDMKEMKENEMKPSEARVPQLSSLELRRYFHRIDNFLKEKKYSDCAWEIVRVEIRRALSYFTKFTALFRRK

SEQ ID NO: 74 - protein, 180 aa, Artificial
  synthetic
LDCNLLNVHLRRVTRQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 75 - protein, 180 aa, Artificial
  synthetic
LDCNLLNVHLRRVTRQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 76 - protein, 180 aa, Artificial
  synthetic
LDCNLLNVHLRRVTRQNLRHLSSMSNSFPVECLRENIAFELPQEFLQYTQPMKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 77 - protein, 180 aa, Artificial
  synthetic
LDCNLLNVHLRRVTRQNLRHLSSMSNSFPVECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 78 - protein, 180 aa, Artificial
  synthetic
LDCNLLNVHLRRVTRQNLRHLSSMSNSFPVECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 79 - protein, 180 aa, Artificial
  synthetic
LDCNLLNVHLRRVTRQNLRHLSSMSNSFPVECLRENNAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 80 - protein, 180 aa, Artificial
  synthetic
LDCNLLNVHLRRVTWQNLRHLSSMSNSFPVECLRENIAFELPQEFLQQTQPNKRDIKKAFYEMSLQAFNIFSQHTSKAWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 81 - protein, 180 aa, Artificial
  synthetic
LDCNLLNVHLRRVTWQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRCLYYFYKFTALFRRK

SEQ ID NO: 82 - protein, 180 aa, Artificial
  synthetic
LDCNLLNVHLRRVTWQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 83 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTFKYWKERHLKQIQTGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 84 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRAEIRRCLYYFYKFTALFRRK

SEQ ID NO: 85 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 86 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRCLYYFYKFTALFRRK

SEQ ID NO: 87 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 88 - protein, 180 aa, Artificial
  synthetic
LDCNLLNVHLRRVTWQNLRHLSSMSNSFPVECLRENNAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 89 - protein, 180 aa, Artificial
  synthetic
LDCNLLNVHLRRVTWQNLRHLSSMSNSQPRECLRENNAFELPQEFLQYTQPMKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 90 - protein, 152 aa, Homo sapiens
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGMDPIVTVKKYFQGIYDYLQEKGYSDCAWEIVRVEMMRALTVSTTLQKRLTK